SEQUENCE LISTING (49 sequences)

SEQ ID NO: 1 - DNA, 762 bp, Viscum album
catatgtacgaacgtatccgtctgcgtgttacccaccagaccaccggtgaagaatatttccggttcatcacgcttctccgagattatgtctcaagcggaagcttttccaatgagataccactcttgcgtcagtctacgatccccgtctccgatgcgcaaagatttgtcttggtggagctcaccaaccaggggggagactcgatcacggccgccatcgacgttaccaatctgtacgtcgtggcttaccaagcaggcgaccaatcctactttttgcgcgacgcaccacgcggcgcggaaacgcatctcttcaccggcaccacccgatcctctctcccattcaacggaagctaccctgatctggagcgatacgccggacatagggaccagatccctctcggtatagaccaactcattcaatccgtcacggcgcttcgttttccgggcggcagcacgcgtacccaagctcgttcgattttaatcctcattcagatgatctccgaggccgccagattcaatcccatcttatggagggctcgccaatacattaacagtggggcgtcatttctgccagacgtgtacatgctggagctggagacgagttggggccaacaatccacgcaagtccagcattcaaccgatggcgtttttaataacccaattcggttggctataccccccggtaacttcgtgacgttgaccaatgttcgcgacgtgatcgccagcttggcgatcatgttgtttgtatgcggagagcgcccgagt

SEQ ID NO: 2 - protein, 252 aa, Viscum album
MYERIRLRVTHQTTGEEYFRFITLLRDYVSSGSFSNEIPLLRQSTIPVSDAQRFVLVELTNQGGDSITAAIDVTNLYVVAYQAGDQSYFLRDAPRGAETHLFTGTTRSSLPFNGSYPDLERYAGHRDQIPLGIDQLIQSVTALRFPGGSTRTQARSILILIQMISEAARFNPILWRARQYINSGASFLPDVYMLELETSWGQQSTQVQHSTDGVFNNPIRLAIPPGNFVTLTNVRDVIASLAIMLFVCGERP

SEQ ID NO: 3 - DNA, 828 bp, Viscum album
aggcctgtgatagccgatgatgttacatgtagtgcttcggaacctacggtgcggattgtgggtcgaaatggcatgtgcgtggacgtccgagatgacgatttccgcgatggaaatcagatacagttgtggccctccaagtccaacaatgatccgaatcagttgtggacgatcaaaagggatggaaccattcgatccaatggcagctgcttgaccacgtatggctatactgctggcgtctatgtgatgatcttcgactgtaatactgctgtgcgggaggccactctttggcagatatggggcaatgggaccatcatcaatccaagatccaatctggttttggcagcatcatctggaatcaaaggcactacgcttacggtgcaaacactggattacacgttgggacagggctggcttgccggtaatgataccgccccacgcgaggtgaccatatatgggttcagggacctttgcatggaatcaaatggagggagtgtgtgggtggagacgtgcgtgagtagccaaaagaaccaaagatgggctttgtacggggatggttctatacgccccaaacaaaaccaagaccaatgcctcacctgtgggagagactccgtttcaacagtaatcaatatagttagctgcagcgctggatcgtctgggcagcgatgggtgtttaccaatgaaggggccattttgaatttaaagaatgggttggccatggatgtggcgcaagcaaatccaaagctccgccgaataatcatctatcctgccacaggaaaaccaaatcaaatgtggcttcccgtgccaggtggatatcactagtaaggatcc

SEQ ID NO: 4 - protein, 267 aa, Viscum album
DDVTCSASEPTVRIVGRNGMCVDVRDDDFRDGNQIQLWPSKSNNDPNQLWTIKRDGTIRSNGSCLTTYGYTAGVYVMIFDCNTAVREATLWQIWGNGTIINPRSNLVLAASSGIKGTTLTVQTLDYTLGQGWLAGNDTAPREVTIYGFRDLCMESNGGSVWVETCVSSQKNQRWALYGDGSIRPKQNQDQCLTCGRDSVSTVINIVSCSAGSSGQRWVFTNEGAILNLKNGLAMDVAQANPKLRRIIIYPATGKPNQMWLPVPGGYH

SEQ ID NO: 5 - DNA, 72 bp, Viscum album
cgcccgagttcctctgaggtgcgctattggccgctggtcataaggcctgtgatagccgatgatgttacatgt

SEQ ID NO: 6 - protein, 17 aa, Viscum album
SSSEVRYWPLVIRRVIA

SEQ ID NO: 7 - DNA, 756 bp, Viscum album
tacgaacgtatccgtctgcgtgttacccaccagaccaccggtgaagaatatttccggttcatcacgcttctccgagattatgtctcaagcggaagcttttccaatgagataccactcttgcgtcagtctacgatccccgtctccgatgcgcaaagatttgtcttggtggagctcaccaaccaggggggagactcgatcacggccgccatcgacgttaccaatctgtacgtcgtggcttaccaagcaggcgaccaatcctactttttgcgcgacgcaccacgcggcgcggaaacgcatctcttcaccggcaccacccgatcctctctcccattcaacggaagctaccctgatctggagcgatacgccggacatagggaccagatccctctcggtatagaccaactcattcaatccgtcacggcgcttcgttttccgggcggcagcacgcgtacccaagctcgttcgattttaatcctcattcagatgatctccgaggccgccagattcaatcccatcttatggagggctcgccaatacattaacagtggggcgtcatttctgccagacgtgtacatgctggagctggagacgagttggggccaacaatccacgcaagtccagcattcaaccgatggcgtttttaataacccaattcggttggctataccccccggtaacttcgtgacgttgaccaatgttcgcgacgtgatcgccagcttggcgatcatgttgtttgtatgcggagagcggccatct

SEQ ID NO: 8 - protein, 252 aa, Viscum album
YERIRLRVTHQTTGEEYFRFITLLRDYVSSGSFSNEIPLLRQSTIPVSDAQRFVLVELTNQGGDSITAAIDVTNLYVVAYQAGDQSYFLRDAPRGAETHLFTGTTRSSLPFNGSYPDLERYAGHRDQIPLGIDQLIQSVTALRFPGGSTRTQARSILILIQMISEAARFNPILWRARQYINSGASFLPDVYMLELETSWGQQSTQVQHSTDGVFNNPIRLAIPPGNFVTLTNVRDVIASLAIMLFVCGERPS

SEQ ID NO: 9 - DNA, 789 bp, Viscum album
gatgatgttacctgcagtgcttcggaacctacggtgcggattgtgggtcgaaatggcatgtgcgtggacgtccgagatgacgatttccgcgatggaaatcagatacagttgtggccctccaagtccaacaatgatccgaatcagttgtggacgatcaaaagggatggaaccattcgatccaatggcagctgcttgaccacgtatggctatactgctggcgtctatgtgatgatcttcgactgtaatactgctgtgcgggaggccactctttggcagatatggggcaatgggaccatcatcaatccaagatccaatctggttttggcagcatcatctggaatcaaaggcactacgcttacggtgcaaacactggattacacgttgggacagggctggcttgccggtaatgataccgccccacgcgaggtgaccatatatgggttcagggacctttgcatggaatcaaatggagggagtgtgtgggtggagacgtgcgtgagtagccaaaagaaccaaagatgggctttgtacggggatggttctatacgccccaaacaaaaccaagaccaatgcctcacctgtgggagagactccgtttcaacagtaatcaatatagttagctgcagcgctggatcgtctgggcagcgatgggtgtttaccaatgaaggggccattttgaatttaaagaatgggttggccatggatgtggcgcaagcaaatccaaagctccgccgaataatcatctatcctgccacaggaaaaccaaatcaaatgtggcttcccgtgcca

SEQ ID NO: 10 - protein, 263 aa, Viscum album
DDVTCSASEPTVRIVGRNGMCVDVRDDDFRDGNQIQLWPSKSNNDPNQLWTIKRDGTIRSNGSCLTTYGYTAGVYVMIFDCNTAVREATLWQIWGNGTIINPRSNLVLAASSGIKGTTLTVQTLDYTLGQGWLAGNDTAPREVTIYGFRDLCMESNGGSVWVETCVSSQKNQRWALYGDGSIRPKQNQDQCLTCGRDSVSTVINIVSCSAGSSGQRWVFTNEGAILNLKNGLAMDVAQANPKLRRIIIYPATGKPNQMWLPVP

SEQ ID NO: 11 - DNA, 48 bp, Viscum album
tcctctgaggtgcgctattggccgctggtcatacgacccgtgatagcc

SEQ ID NO: 12 - protein, 16 aa, Viscum album
SSEVRYWPLVIRPVIA

SEQ ID NO: 13 - DNA, 94 bp, Artificial Sequence
  Synthetic gene encoding amino acids 53-78 of human P2 protein
gtaccgggtggcggtcgtaccgaatccaccttcaaaaacaccgaaatctccttcaaactgggtcaggaattcgaagaaaccaccgctgacaact

SEQ ID NO: 14 - protein, 26 aa, Artificial Sequence
  Amino acids 53-78 of human P2 protein
RTESTFKNTEISFKLGQEFEETTADN

SEQ ID NO: 15 - DNA, 75 bp, Artificial Sequence
  Figure 20 Synthetic linker cassette for providing modularity at the 3′ end of rMLB delta 1 alpha 1 beta
caccggtaaaccgaaccagatgtggctgccggtaccgtagtaacgctcctcgtcgacctagtaaggatccctcga

SEQ ID NO: 16 - protein, 12 aa, Artificial Sequence
  Amino acid sequence encoded by portion of SEQ ID NO 15
TGKPNQMWLPVP

SEQ ID NO: 17 - DNA, 82 bp, Artificial Sequence
  Fig. 21 Synthetic linker cassette for providing modularity at the 3′end of rMLB Delta 1 alpha 1 beta 2 gamma with affinity module (“His-Tag”).
ccggtaaaccgaaccagatgtggctgccggtaccgggtggtggatatcatcaccaccatcaccactagtaactcctcggatc

SEQ ID NO: 18 - protein, 21 aa, Artificial Sequence
  Amino acid sequence encoded by a protion of SEQ ID NO 17
GKPNQMWLPVPGGGYHHHHHH

SEQ ID NO: 19 - DNA, 26 bp, Artificial Sequence
  Codon exchange rMLB D23A
catgtgcgtggccgtccgagatgacg

SEQ ID NO: 20 - DNA, 27 bp, Artificial Sequence
  Fig. 22  Mutagenic oligonucleotides for inactivating carbohydrate binding sites in rMLB. - 1alpha2 (W38A).
cagatacagttggcgccctccaagtcc

SEQ ID NO: 21 - DNA, 61 bp, Artificial Sequence
  Fig. 22Mutagenic oligonucleotides for inactivating carbohydrate binding sites in rMLB. - 1beta (Y68S, Y70S, Y75S, F79S).
gctgcttgaccacgtctggctctactgctggcgtctctgtgatgatctccgactgtaatac

SEQ ID NO: 22 - DNA, 26 bp, Artificial Sequence
  Fig. 22   Mutagenic oligonucleotides for inactivating carbohydrate binding sites in rMLB. - 1beta1 (D235A).
gggttggccatggctgtggcgcaagc

SEQ ID NO: 23 - DNA, 26 bp, Artificial Sequence
  Fig. 22  Mutagenic oligonucleotides for inactivating carbohydrate binding sites in rMLB. - 2gamma2 (Y249A). -
cgaataatcatcgctcctgccacagg

SEQ ID NO: 24 - DNA, 35 bp, Artificial Sequence
  Fig. 22  Mutagenic oligonucleotides for inactivating carbohydrate binding sites in rMLB. - pT7 EcoRV to SspI.
cttcctttttcaatattattgaagcatttatcagg

SEQ ID NO: 25 - DNA, 35 bp, Artificial Sequence
  Fig. 22   Mutagenic oligonucleotides for inactivating carbohydrate binding sites in rMLB. - pT7 SspI to EcoRV. -
cttcctttttcgatatcattgaagcatttatcagg

SEQ ID NO: 26 - DNA, 40 bp, Artificial Sequence
  Fig. 23 Mutagenic oligonucleotides for constructing modular ITF gene cassettes. - pT7 Delta NdeI to StuI.
ctttaagaaggagatatacaggcctacgagaggctaagac

SEQ ID NO: 27 - DNA, 33 bp, Artificial Sequence
  Fig. 23  Mutagenic oligonucleotides for constructing modular ITF gene cassettes. - rMLB silent NheI.
gttacctgcagtgctagcgaacctacggtgcgg

SEQ ID NO: 28 - DNA, 32 bp, Artificial Sequence
  Fig. 23 Mutagenic oligonucleotides for constructing modular ITF gene cassettes. - rMLA Delta AgeI.
cccaccagaccaccggcgaagaatatttccgg

SEQ ID NO: 29 - DNA, 40 bp, Artificial Sequence
  Fig. 23      Mutagenic oligonucleotides for constructing modular ITF gene cassettes.
gtttgtatgcggagagcgtccctcgagctctgaggtgcgc

SEQ ID NO: 30 - DNA, 43 bp, Artificial Sequence
  Fig. 23 Mutagenic oligonucleotides for constructing modular ITF gene cassettes. - rMLB Delta EcoNI to AgeI.
ccgaataatcatcgctccggccaccggtaaaccaaatcaaatg

SEQ ID NO: 31 - DNA, 11 bp, Artificial Sequence
  Flanking region of the ProML gene cassette in expression vector pT7ProML
tacatatgtac

SEQ ID NO: 32 - DNA, 20 bp, Artificial Sequence
  Flanking region of the ProML gene cassette in expression vector pT7ProML
ccatgataaggatcctctag

SEQ ID NO: 33 - DNA, 9 bp, Artificial Sequence
  Flanking region of the IML gene cassette in expression vector PIMML-02-P
caggcctac

SEQ ID NO: 34 - DNA, 34 bp, Artificial Sequence
  Flanking region of the IML gene cassette in expression vector PIML-02-P
cactagtaactcctcggatcctctagagtcgacc

SEQ ID NO: 35 - protein, 4 aa, Artificial Sequence
  Modulator module peptide
KDEL

SEQ ID NO: 36 - protein, 4 aa, Artificial Sequence
  Modulator module peptide
HDEL

SEQ ID NO: 37 - protein, 16 aa, Artificial Sequence
  Portion of the ML propeptide
SSEVRYWPLVIRRVIA

SEQ ID NO: 38 - protein, 13 aa, Artificial Sequence
  A degredation product of mylein basic protein
VHFFKNIVTPRTP

SEQ ID NO: 39 - DNA, 34 bp, Artificial Sequence
  Figure 1.a bFGF specific primer for PCR amplification of the bFGF gene (5′ to 3′)
atattgtcgaccatatggcagccgggagcatcac

SEQ ID NO: 40 - DNA, 32 bp, Artificial Sequence
  Figure 1.a bFGF specific primer for PCR amplification of the bFGF gene (3′ to 5′)
gaaggttacagacgattctcggtatacttaat

SEQ ID NO: 41 - DNA, 15 bp, Artificial Sequence
  Figure 1.b DNA sequence encoding C-terminal processing sequence of bFGF
tctgctaagagccat

SEQ ID NO: 42 - protein, 5 aa, Artificial Sequence
  Figure 1.b Amino acid sequence of C-terminal processing sequence of bFGF
SAKSH

SEQ ID NO: 43 - DNA, 756 bp, Viscum album; identical to SEQ ID NO: 7

SEQ ID NO: 44 - protein, 252 aa, Viscum album; identical to SEQ ID NO: 8

SEQ ID NO: 45 - DNA, 789 bp, Viscum album; identical to SEQ ID NO: 9

SEQ ID NO: 46 - protein, 263 aa, Viscum album; identical to SEQ ID NO: 10

SEQ ID NO: 47 - DNA, 48 bp, Viscum album; identical to SEQ ID NO: 11

SEQ ID NO: 48 - protein, 10 aa, Artificial Sequence
  Figure 21  Portion of amino acid sequence encoded by synthetic gene fragment
GGGYHHHHHH

SEQ ID NO: 49 - DNA, 78 bp, Artificial Sequence
  Fig. 21 Synthetic linker cassette for providing modularity at the 3′ end of rMLB delta 1 alpha 1 beta 2 gamma with affinity module (“His-Tag”).
atttggcttggtctacaccgacggccatggcccaccacctatagtagtggtggtagtggtgatcattgaggagcctag

(19) United States
(12) Patent Application Publication
(10) Pub. No.: US 2008/0227157 A1
(43) Pub. Date: Sep. 18, 2008

(54) Nucleic acids encoding fusion proteins based on ribosome-inactivating proteins of the mistletoe Viscum Album

(75) Inventors: Jurgen Eck, Heppenheim (DE); Arno Schmidt, Buttelborn (DE); Holger Zinke, Bickenbach (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Cytavis BioPharma Gmbh, Hamburg (DE)

(21) Appl. No.: 11/803,114

(22) Filed: May 11, 2007

Related U.S. Application Data
(63) Continuation of application No. 09/347,064, filed on Jul. 2, 1999, now abandoned.
Application No. 09/347,064 is a continuation of application No. PCT/EP98/00009, filed on Jan. 2, 1998.

(30) Foreign Application Priority Data
Jan. 2, 1997 (EP) EP97100012.0

(51) Int. Cl. C12P 21/04; C12N 15/11; C12N 15/00; C12N 1/20

(52) U.S. Cl. 435/69.7; 536/23.4; 435/320.1; 435/252.3; 435/252.31; 435/252.33; 435/252.35; 435/254.3; 435/254.23

(57) ABSTRACT

The invention relates to nucleic acid molecules which encode fusion proteins which contain as components at least one effector module, a processing module and a targeting module. The nucleic acid molecules according to the invention preferably also encode a modulator module and/or an affinity module. The invention furthermore relates to vectors containing these nucleic acid molecules, hosts transformed with the vectors according to the invention, fusion proteins encoded by nucleic acids according to the invention or produced by the hosts according to the invention as well as to medicaments containing the polypeptides or vectors according to the invention. The invention thus also concerns corresponding processes, uses and kits.

### CROSS REFERENCE TO RELATED APPLICATION

This application is a continuation of U.S. patent application Ser. No. 09/347,064, filed Jul. 2, 1999, which is a continuation of International Application PCT/EP98/00009, filed Jan. 2, 1998, the disclosures of which are hereby incorporated herein by reference.

### BACKGROUND OF THE INVENTION

During the last few years medical research discovered a wide range of diseases that are associated with the change or degeneration of exogeneous cells which is reflected, e.g., in a cell-specific or modified set of receptors. A widely used strategy for developing therapeutical approaches is based on the principle to couple a cytocidal substance which per se is not capable of penetrating the cell's core with a second non-toxic substance which is capable of penetrating the cell's core by binding to a surface protein. The more cell-type specific the targeting molecule the more selectively pathogenic cells can be destroyed without damaging healthy cells. Such cell-type specific toxic fusion proteins are used in the form of so-called immunotoxins and mitotoxins (Vitetta et al., 1987; Lambert et al., 1988; Lappi et al., 1990; Pastan et al., 1991; Ramakrishnan et al., 1992; Pastan et al., 1992; Brinkmann, 1996) to selectively destroy tumor cells.

Known examples of cytocidal components are the bacterial toxins diphterotoxin (Collier, 1988), Pseudomonas exotoxin (Pastan et al., 1989) and tetanus toxin (Brinkmann, 1996), as well as plant-derived ribosome-inactivating proteins (RIP; Barbieri et al., 1993). The plant toxins are differentiated in type I RIPs such as gelonin or saporin which consist of a single toxic domain, and type II RIPs (including mistletoe lectin) which have a second domain with sugar-binding properties (Stirpe et al., 1992; Barbieri et al., 1993). The best-known representative of the latter group is ricin. For the toxic effect to develop, a complex uptake and processing pathway is required: after receptor-mediated uptake, transport across clathrin-coated vesicles in endosomes (Nicolson, 1974) the toxin component is processed/released from the fusion protein as prerequisite for translocation into the cytoplasm. There, the toxin develops its toxic effect and destroys the cell. Mistletoe lectin has been described as potent inducer of apoptosis (Janssen et al, 1996). This property, in turn, is associated with the interaction of A and B chain, with RIP activity being crucial. Depending on the concentration and point in time, the cytotoxicity of mistletoe lectin of apoptotic or necrotic nature. If high concentrations or dosages are used, necrotic cell death can be observed. The same is true for moderately toxic concentrations which are applied for a time period exceeding 24 hrs. In a period of few hours or at low concentrations the nature of the ML-induced cell death is apoptotic; this observation was made for various cell types (MOLT-4, THP-1, PBMC; Möckel et al, 1997).

One of the first attempts at linking a toxin with a targeting molecule was the chemical coupling via thioether (Masuho et al., 1982). In some cases, however, due to the irreversible coupling the toxin is inactivated (Vitetta et al., 1993). This is why usually coupling agents are used which lead to a coupling via a disulfide bond such as N-succinimidyl-3-(2-pyridyldithio)propionate (SPDP; Carlsson et al., 1978; Jansen et al., 1982), risking, however, that components that are coupled via disulfide bonds possess a relatively low in vivo stability. Also, along with this protein-chemical modification often a substantial loss of activity could be observed (Thorpe et al., 1981; Battelli et al., 1990; Bolognesi et al., 1992). Another major drawback of the chemical coupling is the generation of an inhomogeneous mixture of substances which entails the use of complicated methods for enriching the desired product (Pastan, 1992).

In order to avoid the problem of chemical coupling, researchers have begun to develop bifunctional antibodies that can bind to a toxin with one binding site and to a target cell with the other (Milstein et al., 1983; Webb et al., 1985; Glennie et al., 1988). While this made it possible for the toxin to be easily released during internalization, a partial dissociation of the complexes and hence a partial unspecific toxicity caused by the toxins could be observed already during circulation in the blood. Furthermore, the process for producing the specific antibodies is very complicated. Due to the high molecular weight of these constructs the immunogenic potential is increased as well as tumor penetration deteriorated (Brinkmann, 1996). Also, production of the bispecific antibodies is a very time-consuming process.

Modern molecular-biological methods have made it possible to clone toxic proteins such as diphterotoxin, Pseudomonas exotoxin, ricin or saporin (Greenfield et al., 1983; Gary et al., 1984; Lamb et al., 1985; Benatti et al., 1989) and thus to make them accessible to genetic fusions with target domains. The use of recombinant bacterial toxins has had medical successes regarding their effectiveness, however, it still is problematic since large parts of the population have been immunized by vaccination and therefore possess neutralizing antibodies against the toxin component (Brinkmann, 1996). It is therefore advantageous to use plant toxins such as mistletoe lectin or ricin. For a toxic effect to develop (of type II RIPs or recombinant fusion proteins), however, it is crucial that the toxin/the toxin component is intracellularly released (Barbieri et al., 1993). For example, the A chain of ricin (ricin A) was used to recombinantly construct mitotoxins, whereby two recombinant IL2-ricin A fusion proteins were constructed which differed in the choice of the linker sequence. The construct with the intracellularly protease-sensitive diphterotoxin loop is cytotoxic vis-à-vis CTLL-2 cells while the second variant with a not intracellularly processable linker sequence is not cytotoxic (Cook et al., 1993). The authors make use of the protease-sensitive sequences that naturally occur in bacterial toxins. It is not or only possible to a limited extent to transfer the findings to other toxins as effector module. For toxins other than those described by Cook new possibilities of activation/processing must be created. Naturally, type II-RIPs are synthesized in the plant in form of RIP-inactive pre-pro-proteins and then processed to mature toxins in specific cell compartments (Lord, 1985). What could be shown was the translation of pro-ricin-mRNA in Xenopus oocytes (Westby et al., 1992). However, no indications for an in vivo activation of the pro-proteins could be found which excludes the use of a recombinant proricin as toxin (Richardson et al., 1989). On the basis of this and other results it has so far been started from the assumption that the processing of the pro-sequences of the type I-RIPs is brought about by specific plant proteases and assumed that this principle also applies to the mistletoe lectin (Hara-Nishimura et al., 1991).

During the search for a suitable toxin as effective ingredient in immunotoxins it was mainly ricin that was examined. On the basis of the A domain of the type II-RIPs ricin (ricin A) a number of immunotoxins was prepared and tested for cancer therapy (Spitler et al., 1987; Shen et al., 1988; Byers et al., 1989; Vitetta et al., 1991). However, it is a disadvantageous property of ricin A that it may also unspecifically penetrate cells so that it produces grave side-effects such as the “vascular leak syndrome” in most patients (Gould et al., 1989; Soer-Rodriguez et al., 1993). In another study efforts at using saporin as component of immunotoxins have been described. This study deals with the comparison of biochemical and recombinant production methods of immuno- or mitotoxins, wherein the type I-RIP saporin was coupled to the mitogen “bFGF” both chemically and by gene fusion (Lappi et al., 1994). The substances produced by different methods exhibit the same anti-tumor effect in in vitro and in in vivo studies. However, the production of the recombinant substance is less problematic by far. It is, however, true that the intracellular release of the toxin was only made possible by the not generalizable condition that the targeting molecule bFGF used possesses a protease sensitive cleavage site. Therefore, it does not seem possible to broadly use the data provided by the authors on a wide range of target cells of interest.

Sun et al. (1997) describe a chemical-covalent conjugate consisting of the Cholera Toxin B subunit (CTB) and the Myelin Basic Protein (MBP), with which EAE, the animal model of MS, can be effectively suppressed at an oral application of 50 μg protein. The conjugate with the toxin is 50 to 100-fold more toxic than the antigen MBP alone. The two components MBP and CTB were each isolated from the natural source. This approach shows that in principle a toxin may be transported to the site where it shall be effective, i.e. to the target cells, by way of antigen recognition. However, the mode of production of the conjugates involves the difficulties described above for the chemical coupling and the limited availability and consistent purity of the components.

Fusion proteins have been described for their use as vaccines (Price, 1996). For this purpose, antigens were coupled to GM-CSF in the yeast expression system to stimulate the immune response, with the individual antigen always being coupled to the C terminus of the GM-CSF, optionally with an intervening linker. The fusion proteins described are limited regarding their use to the stimulation of antigen-presenting cells by the growth factor GM-CSF and regarding their preparation to the expression in yeast.

Better et al. (1995) describe fusion proteins from humanized antibodies and the RIP gelonin. Using these fusion proteins, the authors were able to target CD5-positive T and B cells. The toxicities differed widely, depending on the orientation and nature of the components. PBMC from 2 different donors were insensitive to antibody ricin A chain fusion proteins, but sensitive to those fusion proteins with gelonin as toxin. This finding illustrates that the choice of a suitable toxin can be decisive for the effectiveness of an immune fusion protein. The approach taken by Better et al., however, requires that antibody genes encoding those antibodies recognizing a specific determinant of target cells are available. These requirements, however, are exactly not necessarily met in the case of autoreactive T cells since they rather are defined by their antigen recognition.

Another approach taken in order to render autoreactive T cells harmless by presenting to them their specific antigen is based on the technique of loading MHC molecules isolated from spleen cell membranes with antigen fragments such as MBP, HSP and acetylcholine receptor peptides (Spack et al., 1995). The presentation of the respective antigen without co-stimulatory signals renders the T cells anergic, i.e., the binding of the antigen does not induce proliferation but the cells remain in a quiescent state. In the animal model of the autoimmune disease Myastenia Gravis a progression of the disease could be avoided by using such a protein complex. The disadvantage of the concept of anergy induction is that the effect that does not last long since the antigen, which per se is not toxic, does not kill but only temporarily inhibits the cell if administered in low amounts.

There is a general need in the present state of the art for a modular system of suitable effector, processing, modulator, targeting and affinity modules which allows a universal applicability for different medical indications. If the cell populations relevant for a disease, particularly in the field of the immunologically competent cells, are known, it would be desirable to be able to specifically influence or switch them off.

The problem underlying the present invention is therefore to remove the disadvantages known in the art to be involved in the construction of immunotoxins and at the same time to make sure that the immunotoxins develop their toxic effect in a broad range of target cells only intracellularly.

The solution to this problem is provided by the embodiments characterized in the claims.

### BRIEF DESCRIPTION OF THE SEVERAL VIEW OF THE DRAWINGS

FIG. 1.a illustrates construction of a vector for the expression of a type TPE (bFGF-MLA) rML-ITF. As can be seen in the figure, the bFGF gene is amplified using bFGF specific primers (SEQ ID NO: 39 (5′→3′); SEQ ID NO: 40 (3′→5′)) that contain a Nde I restriction site.

FIG. 1.b illustrates a carboxyl-terminal processing sequence of bFGF SEQ ID NO: 41 and the corresponding derived amino acid sequence (SEQ ID NO: 42).

FIG. 1.c depicts an expression vector of the effector module (rMLA).

FIG. 2 depicts vectors for the expression of the modules TPE (bFGF-MLA) and M (rMLB) for the in vitro association.

FIG. 3 illustrates construction of a vector for the expression of a type EPM^{T} (ProML) rML-ITF.

FIG. 4.a is an image of a pair of gels which indicate recombinant production of bFGF-MLA.

FIG. 4.b is an image of a gel which indicates recombinant production of rMLA.

FIG. 5.a is an image of a gel which indicates recombinant production of bFGF-MLA/rMLB (total protein stain).

FIG. 5.b is a gel which indicates recombinant production of bFGF-MLA/rMLB (Western blot analysis).

FIG. 6 is a pair of gels which indicate recombinant production of ProML.

FIG. 7 is a graph which indicates cytotoxicity of bFGF-MLA.

FIG. 8.a is a graph which indicates cytotoxicity of bFGF-MLA/rMLB.

FIG. 8.b is a graph which indicates modulation of the cytotoxicity of bFGF-MLA by rMLB.

FIG. 9.a is a graph which indicates cytotoxicity of ProML.

FIG. 9.b is a graph which indicates cytotoxicity of ProML as compared to rML.

FIG. 10 depicts an exemplary selection of possible combinations of the rML-ITF modules.

FIG. 11.a lists the nucleotide sequence (SEQ ID NO: 1) and derived amino acid sequence (SEQ ID NO: 2) of rMLA.

FIG. 11.b lists the nucleotide sequence (SEQ ID NO: 3) and derived amino acid sequence (SEQ ID NO: 4) of rMLB.

FIG. 11.c lists the nucleotide sequence (SEQ ID NO: 5) and derived amino acid sequence (SEQ ID NO: 6) of the rML-propeptide. The nucleotide sequence of FIG. 11 shows various restriction sites, start and stop codons which the person skilled in the art will remove or modify if necessary for the purpose according to the invention. Such embodiments are shown in FIG. 11.a′-11.c′, described infra.

FIG. 11.a′ lists the nucleotide sequence (SEQ ID NO: 43) and the derived amino acid sequence (SEQ ID NO: 44) of rMLA, recombinant A domain of the mistletoe lectin.

FIG. 11.b′ lists the nucleotide sequence (SEQ ID NO: 45) and the derived amino acid sequence (SEQ ID NO: 46) of rMLB, recombinant B domain of the mistletoe lectin.

FIG. 11.c′ lists the nucleotide sequence (SEQ ID NO: 47) and the derived amino acid sequence (SEQ ID NO: 37) of the rML propeptide.

FIG. 11.d depicts flanking regions (SEQ ID NOs: 31 and 32) of the ProML gene cassette in expression vector pT7ProML.

FIG. 11.e depicts flanking regions (SEQ ID NOs: 33 and 34) of the IML gene cassette in expression vector pIML-02-P.

FIG. 12 is an image of a gel which indicates recombinant production of rML.

FIG. 13 is an image of a gel which indicates recombinant production of rIML (rML Δ1a1β2γ).

FIG. 14 is a graph which indicates cytotoxicity of rIML with inactivated carbohydrate binding sites as compared to rML (wild-type).

FIG. 15 illustrates construction of a vector for the expression of an rML derivative without carbohydrate affinity.

FIG. 16, comprising FIGS. 16.1, 16.2, and 16.3, illustrates construction of modular periplasmic expression systems for the production of ITF-toxins.

FIG. 17 illustrates assembly of ITF toxins on the basis of vectors pIML-03-H and pIML-03-P with specific activity to target cells.

FIG. 18 depicts a vector for the expression of an ITF toxin, specific of a P2-reactive neuritogenic T cell line.

FIG. 19 lists the nucleotide sequence (SEQ ID NO: 13; and the corresponding amino acid sequence; SEQ ID NO: 14) of a synthetic gene cassette encoding amino acids 53 to 78 of the P2 protein.

FIG. 20 lists the nucleotide sequence (SEQ ID NO: 15; and the corresponding amino acid sequence; SEQ ID NO: 16) of a synthetic linker cassette for providing modularity at the 3′ end of rMLB Δ1α1β2γ.

FIG. 21 lists the nucleotide sequence (SEQ ID NOs: 17 and 49; and the corresponding amino acid sequence; SEQ ID NO: 18) of a synthetic linker cassette for providing modularity at the 3′ end of rMLB Δ1α1β2γ with affinity module (“His-Tag”).

FIG. 22 lists the nucleotide sequences of mutagenic oligonucleotides for inactivating carbohydrate binding sites in rMLB:

(i) 1α domain: 1α₁ (SEQ ID NO. 19) and 1α₂ (SEQ ID NO. 20);

(ii) 1β domain: 1β1 (SEQ ID NO. 21);

(iii) 2γ domain: 2γ1 (SEQ ID NO: 22) and 2γ2 (SEQ ID NO: 23);

(iv) pT7 selection primer: pT7 Eco RV→Ssp I (SEQ ID NO: 24) and pT7 Ssp I→Eco RV (SEQ ID NO: 25).

FIG. 23 lists the nucleotide sequences of mutagenic oligonucleotides for the construction of modular ITF gene cassettes:

(i) pT7 Δ Nde→Stu I (SEQ ID NO: 26);

(ii) pT7 Nhe I (SEQ NO: 27);

(iii) pT7 ΔAge I (SEQ ID NO: 28);

(iv) pT7 Ava I (SEQ. ID. NO: 29); and

(v) pT7 IML ΔNde I→Age I (SEQ ID NO: 30).

FIG. 24 is a pair of gels which indicate purification of ITF-P2-C1 on Ni-NTA sepharose under denaturing conditions.

FIG. 25 is a gel which indicates purification of ITF-P2-C1 on Ni-NTA sepharose under physiological conditions.

FIG. 26 is a gel which indicates processing of pITF-P2-C1 during the production in E. coli.

FIG. 27 is a gel which indicates production of ITF by in vitro folding.

FIG. 28, comprising FIGS. 28.a, 28.b, and 28.c, is a trio of FACS analyses of P2-specific T cells after 2 hrs' incubation with ITF-P2-C1.

FIG. 29, comprising FIGS. 29.a, 29.b, 29.c, and 29.d, is a quartet of FACS analyses of P2-specific T cells after 24 hrs' incubation with ITF-P2-C1.

### SUMMARY OF THE INVENTION

The invention relates to nucleic acid molecules which encode fusion proteins which contain as components at least one effector module, a processing module and a targeting module. The nucleic acid molecules according to the invention preferably also encode a modulator module and/or an affinity module. The invention furthermore relates to vectors containing these nucleic acid molecules, hosts transformed with the vectors according to the invention, fusion proteins encoded by nucleic acids according to the invention or produced by the hosts according to the invention as well as to medicaments containing the polypeptides or vectors according to the invention. These medicaments are particularly significant for the therapy of diseases associated with a pathological reproduction and/or increased activity of cell populations. A temporary, periodic and strong proliferation, infiltration and immune activity of cells of the immune system is found in autoimmune diseases and allergies, the specificity of these immune cells being due to their reaction to a particular antigen or allergen. These medicaments may also be advantageously used for treating tumors. The polypeptides and vectors described in the present invention may be used to develop medicaments and to test toxin activity-modulating factors. The invention thus also concerns corresponding processes, uses and kits. The modules, with the exception of the affinity and the targeting module, are preferably encoded by nucleic acids extracted or derived from the mistletoe lectin proprotein coding sequence.

### DETAILED DESCRIPTION OF THE INVENTION

The invention relates to a nucleic acid molecule encoding a fusion protein displaying the following components:
(a) an effector module, which has an intracellular cytotoxic effect;
(b) a processing module, which is covalently linked to the effector module and which displays a recognition sequence for a protease; and
(c) a targeting module which is covalently linked to the processing module and which specifically binds to the surface of a cell, thereby mediating the internalization of the fusion protein in the cell,
   wherein the effector module comprises the mistletoe lectin A chain or a fragment or derivative thereof and/or the processing module comprises the sequence of the mistletoe lectin pro-peptide or a fragment or derivative thereof which is proteolytically cleavable.

According to the invention, the term “module” refers to a peptide which is encoded by a DNA sequence and exhibits certain functional properties. These functional properties are attributable to the primary, secondary and/or tertiary structure of these peptides and relate to biochemical, molecular, enzymatic, cellular and/or physiological functions. A module according to the invention is furthermore characterized in that it displays favorable adapters on the DNA level which easily allow a fusion to other modules and that these adapter sequences do not disadvantageously interfere on the peptide level with the functions of the modules.

In the present invention, the term “fusion protein” is defined such that the nucleic acids according to the invention and the fusion proteins encoded by them are recombinantly produced molecules.

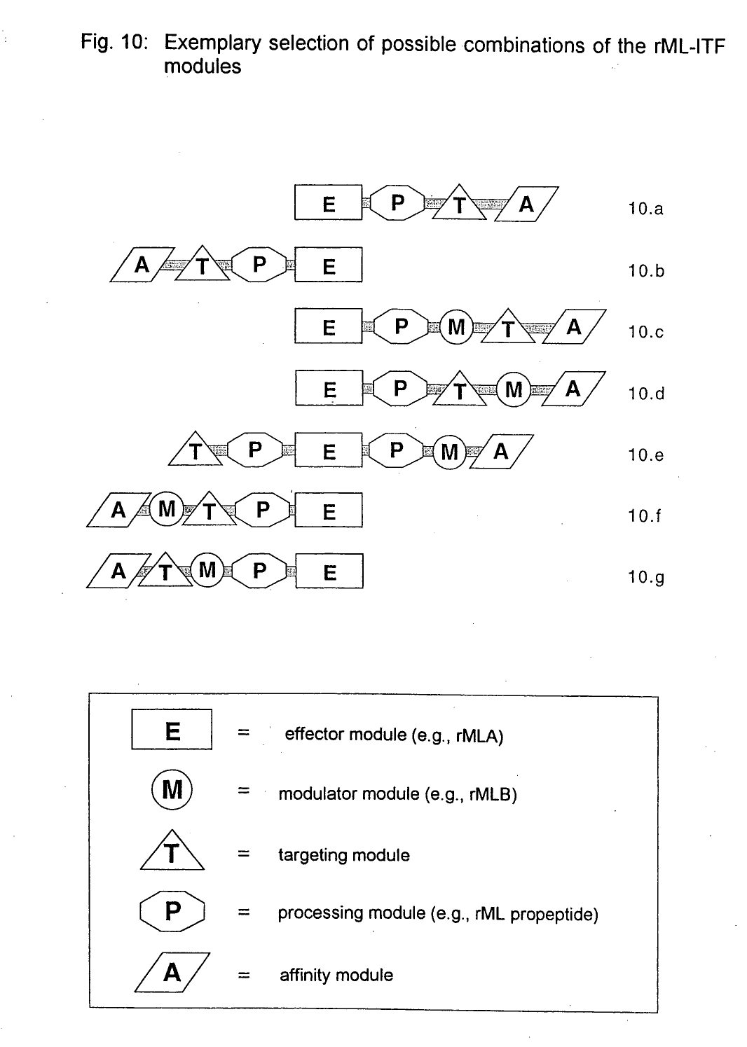

The term “targeting module which is covalently linked to a processing module” is understood in the present invention to also refer to those embodiments in which other modules or sequences covalently intervene between the two aforementioned modules. In this context, reference is made to FIG. 10.c which shows an embodiment according to the invention: There, the targeting module is covalently linked to the processing module via a modulator module. It is important within the meaning of the present invention that the linkage of processing and targeting module, with or without intermediary sequence, is of covalent nature.

According to the invention, the function of the effector module is to kill or to permanently modify the vital processes of the target cells. This function can be triggered by enzymatic activities of the effector module in that physiological intracellular processes are impaired (e.g., metabolic processes, particularly processes of the energy metabolism, molecular-genetic processes, particularly translation, transcription and replication and specific cellular reaction sequences such as, e.g., the induction of apoptotic processes). In any case a target cell is modified via the intracellular activity of the effector module in its physiological status, e.g., its growth behavior, e.g., it is retarded or completely killed and destroyed. A preferred example of a suitable effector module is the recombinant A domain of the mistletoe lectin (rMLA) or an intracellular toxic fragment or derivative thereof. The term “fragment” of a mistletoe lectin A chain is understood in the present invention as a peptide which exhibits part of the amino acid sequence of said chain and exhibits intracellular toxic activity. The toxicity does not have to be on the same level as that of the complete A chain. A fragment can, for example, be generated by proteolytic cleavage of the recombinantly produced A chain or by recombinant manipulation of the A chain encoding nucleic acid and subsequent expression. The person skilled in the art knows on the basis of his general expert knowledge and the teaching of the present invention how to recombinantly produce the fragments mentioned in the present application and later on and how to test them for their activity. The catalytic activity of rMLA resides in the depurination of the 28S rRNA of eukaryotic cells. The use of rMLA as effector module is of particular interest, since in therapeutic dosages it brings about cell death mainly by inducing apoptosis so that in contrast to a necrosis there is no tissue-damaging inflammatory response caused by cell debris and intracellular components. Programmed cell death (apoptosis) inter alia is involved in the regulation of cell populations of the immune system, e.g., also in the elimination of T cells which can be stimulated or “overstimulated” by their specific antigen depending on the concentration. In the case of autoimmune diseases this phenomenon is the natural mechanism for controlling an autoimmune response (termination of an incident) (Schmied et al., 1993) and therefore can be therapeutically used to rush autoreactive T cells into apoptosis by administering specific amounts of the antigen (Gold et al., 1997).

According to the invention, the function of the processing modules is on the one hand to covalently link the effector module to modulator, targeting or affinity modules to a polypeptide chain, which allows to recombinantly produce the fusion proteins. On the other hand, they excel by their content of suitable recognition sequences for proteases, which allows the intracellular release of the effector module in the target cell by the cell's own proteases during receptor-mediated endocytosis in the endosomes and prelysosomes. The processing module of the mistletoe lectin, e.g., in the case of C-terminal fusion to the rMLA, in contrast to the corresponding sequences in propeptides of other plant-derived type II-RIPs such as, e.g., the ricin, surprisingly meets both the requirements for intracellular processing by endosomal proteases of mammalian cells or human cells, as well as rMLA-inactivating properties in a non-processed condition. Preferably, the proteases cleaving the processing module are mammalian proteases. Particularly preferred are proteases of human origin. It is furthermore preferred that these proteases are of intracellular origin.

As targeting modules all molecules on polypeptide basis are understood according to the invention which are capable of allowing access to the fusion protein according to the invention to the cell's core via a specific affinity to a cell surface protein. As target cells particularly immune cells of the blood such as T lymphocytes are useful which can be distinguished via their individual set of receptors by using suitable targeting modules. Proteins, protein fragments or peptides may serve as targeting modules. For example, these peptides could be MHC-binding peptides which could be advantageously used to selectively inactivate clonal T cell lines, for example allergenic T_{H}2 cell lines.

The elucidation of the nucleotide sequence of the mistletoe lectin gene described in the co-pending European patent application with the application no. EP 95109949.8 created the basis for the present invention. The disclosure content of said application is explicitly incorporated into the present application by reference. The recombinant availability of the ProML gene made it possible to generate with a flexible modular concept (exemplarity shown in FIG. 10.a-10.g) new immunotoxin substances with a broad range of target cell specificity expanding surprisingly few efforts. The use of short peptides as targeting modules, which may be particularly used for specifically binding to T cell receptors, allows a direct chemical synthesis of the DNA sequence individually required (which becomes part of the nucleic acid according to the invention), which is substantially less time-consuming than, e.g., the construction of suitable antibodies. Another advantage of the concept according to the invention for producing new highly specific toxins vis-à-vis the construction of immunotoxins via bispecific antibodies is the covalent linkage of the modules via processing modules which prevent an extracellular dissociation of the modules and allow the intracellular release of the toxin. It was furthermore found according to the invention that the natural propeptide of the mistletoe lectin, due to its protease-sensitive properties, which so far have not been reported for the propeptides of other type II-RIPs, is an excellent source for suitable processing modules for the construction of the fusion proteins according to the invention. What is most striking is that processing modules of plant origin are recognized by non-plant proteases, which feature allows their universal use.

The term “plant origin” means in the context of the present invention a peptide sequence which is encoded by a nucleic acid molecule homologous to regions of the plant genome or a component thereof. The homology of the nucleic acid molecules is brought about by hybridization under stringent conditions.

Another advantage of the invention is that when the fusion proteins are used no problems are caused by the various vaccines, which is often the case when immuno- and mitotoxins on the basis of bacterial toxins are used. rMLA as effector module of the fusion proteins according to the invention exhibits improved properties vis-à-vis ricin A which so far has been used most frequently for constructing immunotoxins. A direct comparison shows that chemically coupled MLA-based immunotoxins are more efficient by far than those on the basis of ricin A. Also, ricin A as well as immunotoxins on the basis of ricin carry strong side-effects caused by their unspecific toxicity, which so far have not been reported for MLA.

Another advantage of the fusion proteins according to the invention is the possibility of their recombinant production, which is preferably carried out in E. coli. This preferred embodiment of the fusion proteins according to the invention is thus free of glycosylations and is therefore not bound by the glycoside receptors of the liver cells as is the case with the toxins obtained from plants. This leads to less liver damages with simultaneously prolonged half-times in the blood and thus represents a substantial improvement of the therapeutical possibilities, since plant-derived toxins are mainly glycosylated with terminal mannose residues, which leads to a fast degradation in the liver. A major advantage of the recombinant production of fusion proteins in, e.g., E. coli is that these proteins do not display a glycosylation which reduces the unspecific toxicity of plant toxins on non-parenchymal hepatocytes (Skilleter et al., 1985; Magnusson et al., 1993) and simultaneously prolong the therapeutic half-time (Vitetta et al., 1993). Thus, the use of the fusion protein according to the invention, for example for the specific inactivation of pathological immune cells of the blood, offers a broad range of advantages vis-à-vis the toxins known so far. The enormous advantages of these properties of the fusion proteins according to the invention particularly in the medical sector are evident for the person skilled in the art.

Another important advantage of the fusion proteins is that, compared with conventional immunotoxins, they may have a considerably lower molecular weight, which reduces the danger of immune responses and improves the distribution of the substance in dense cell tissues.

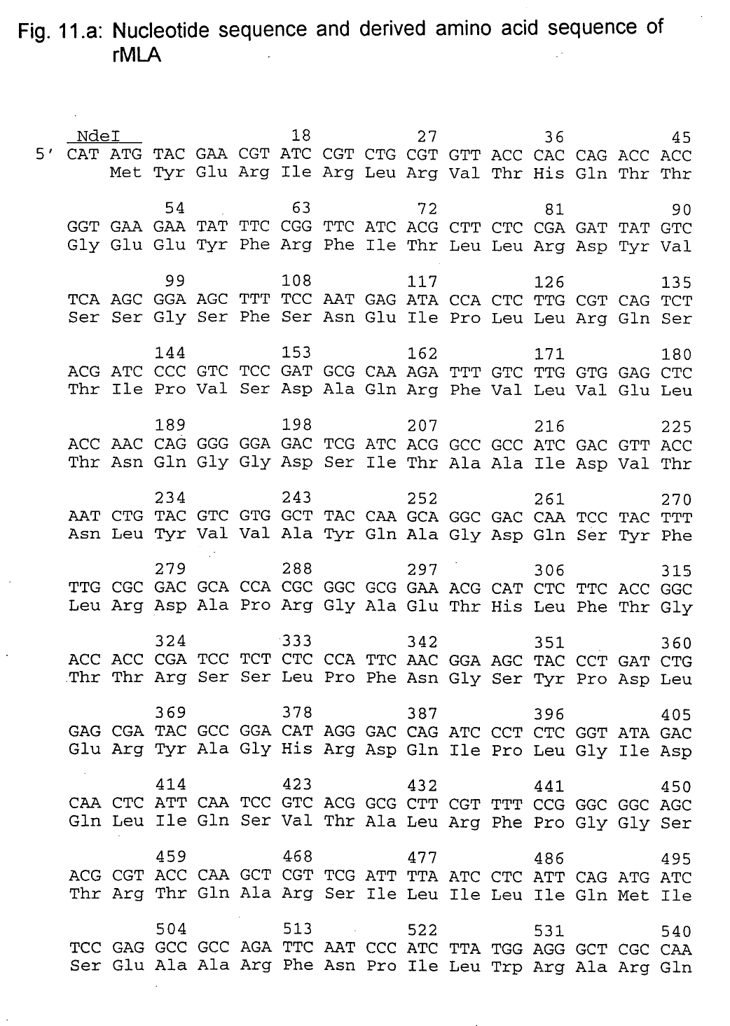

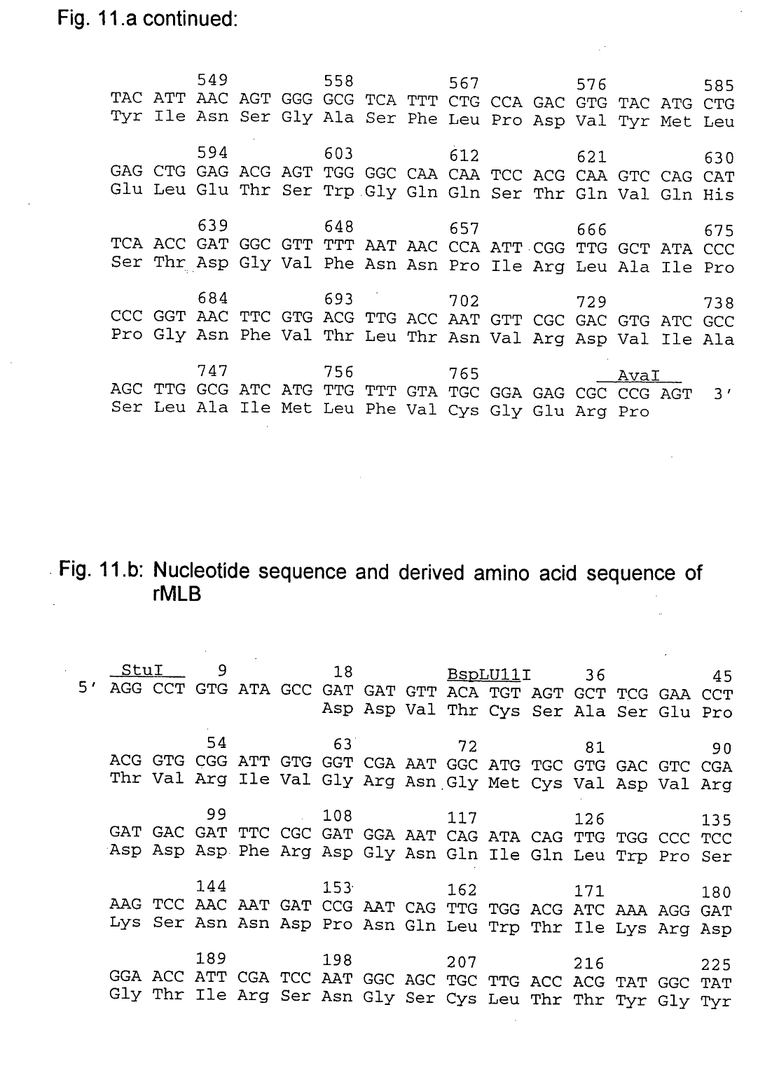

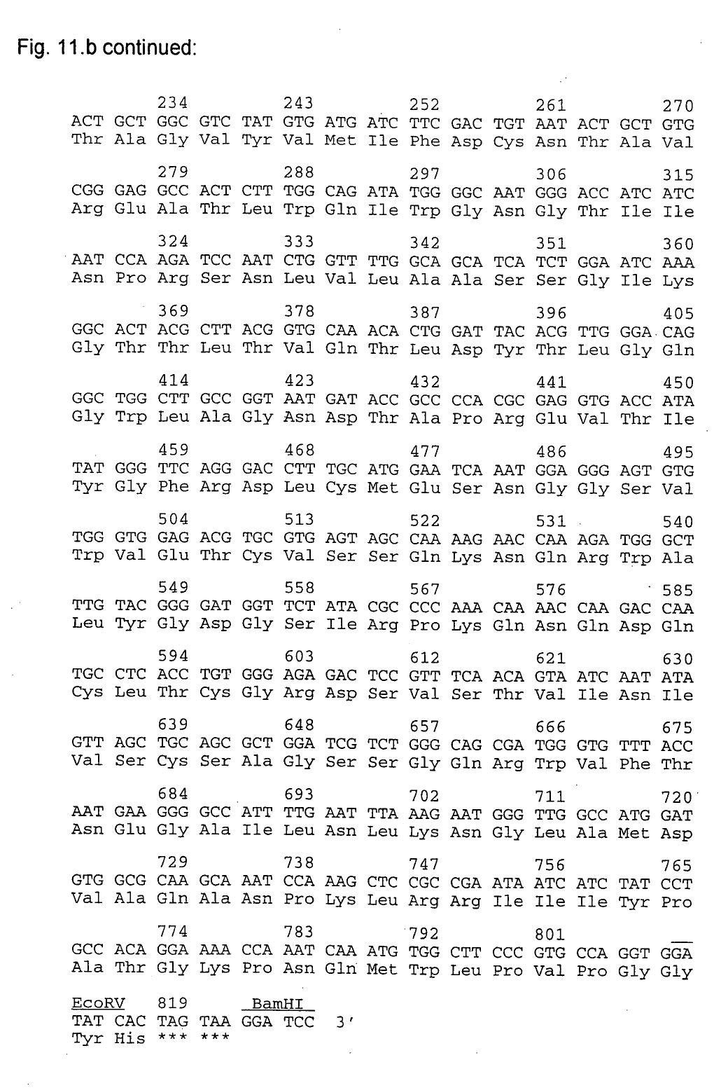

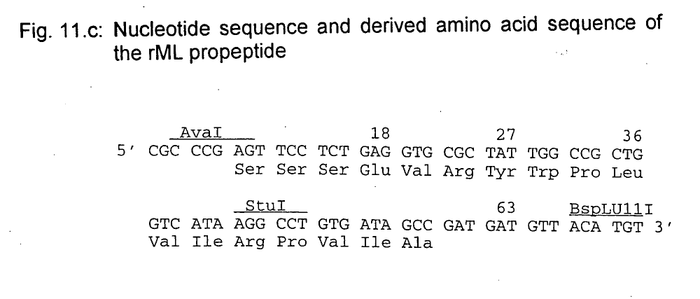

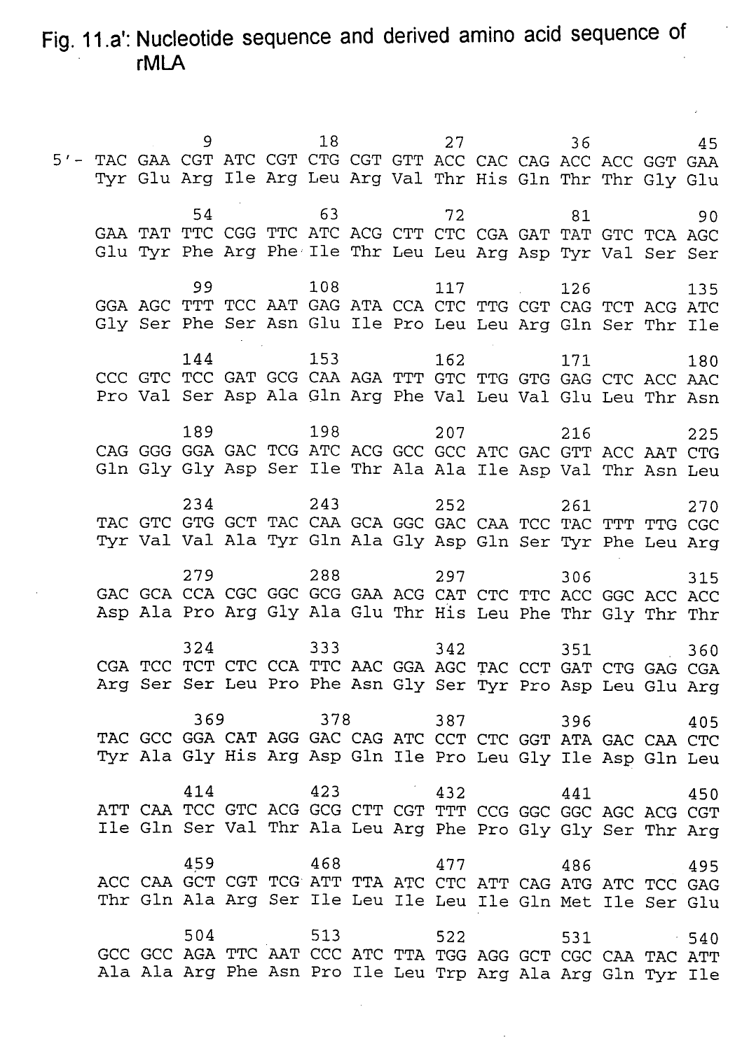

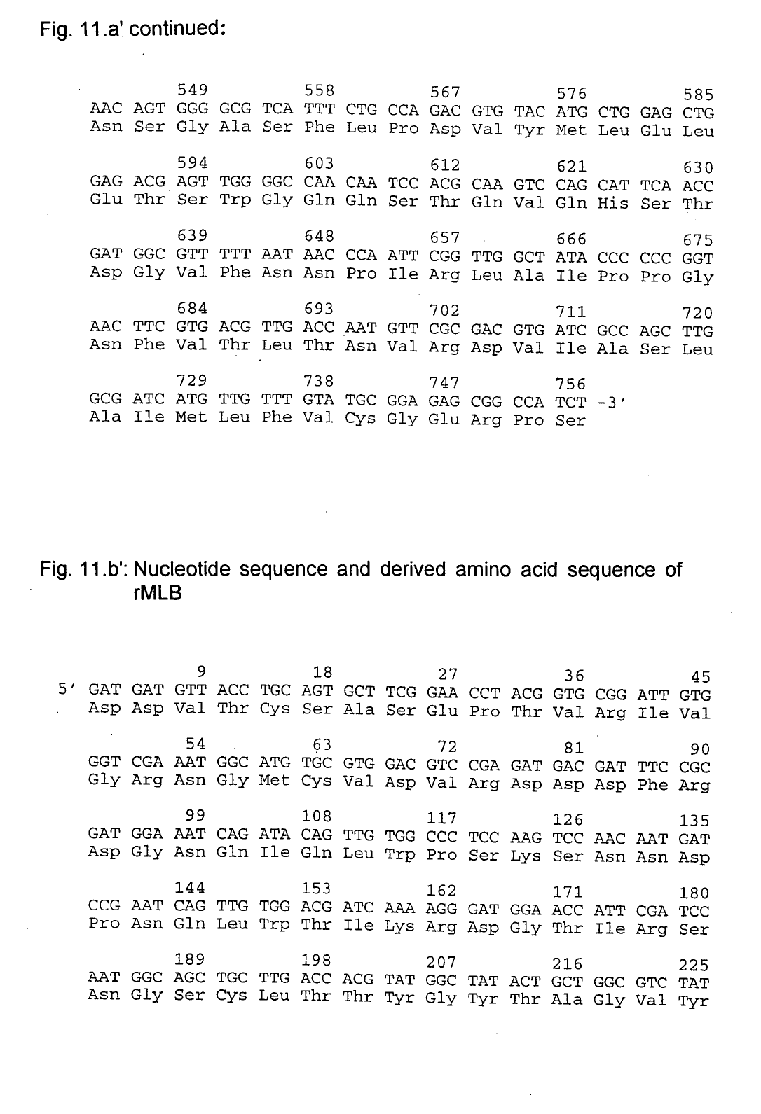

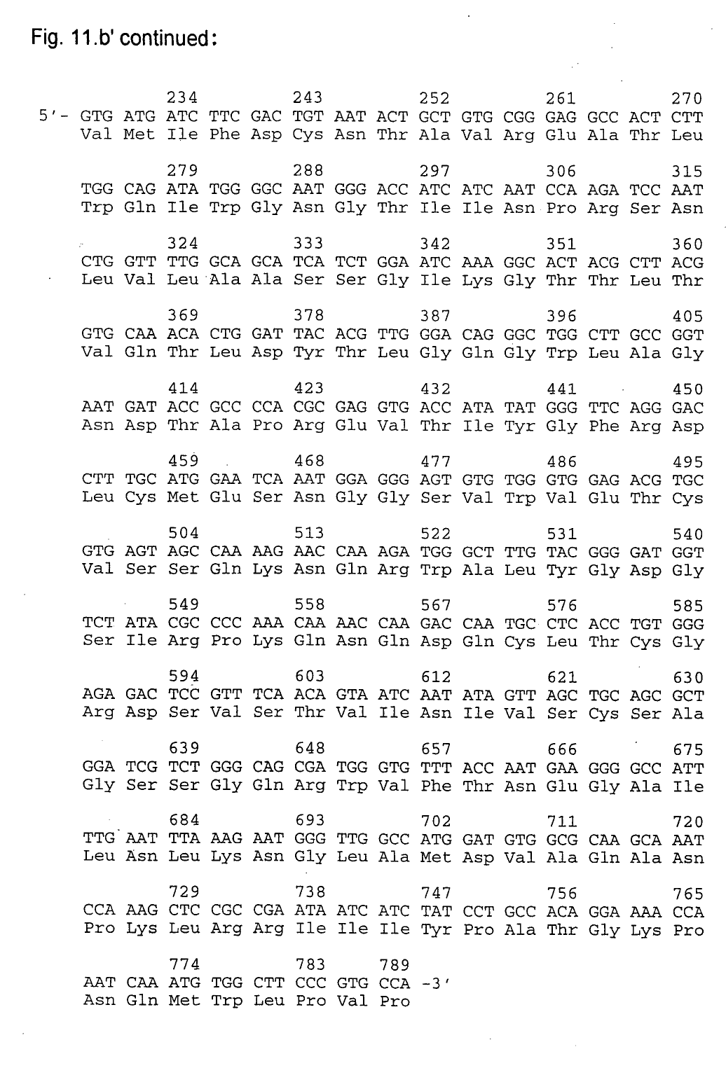

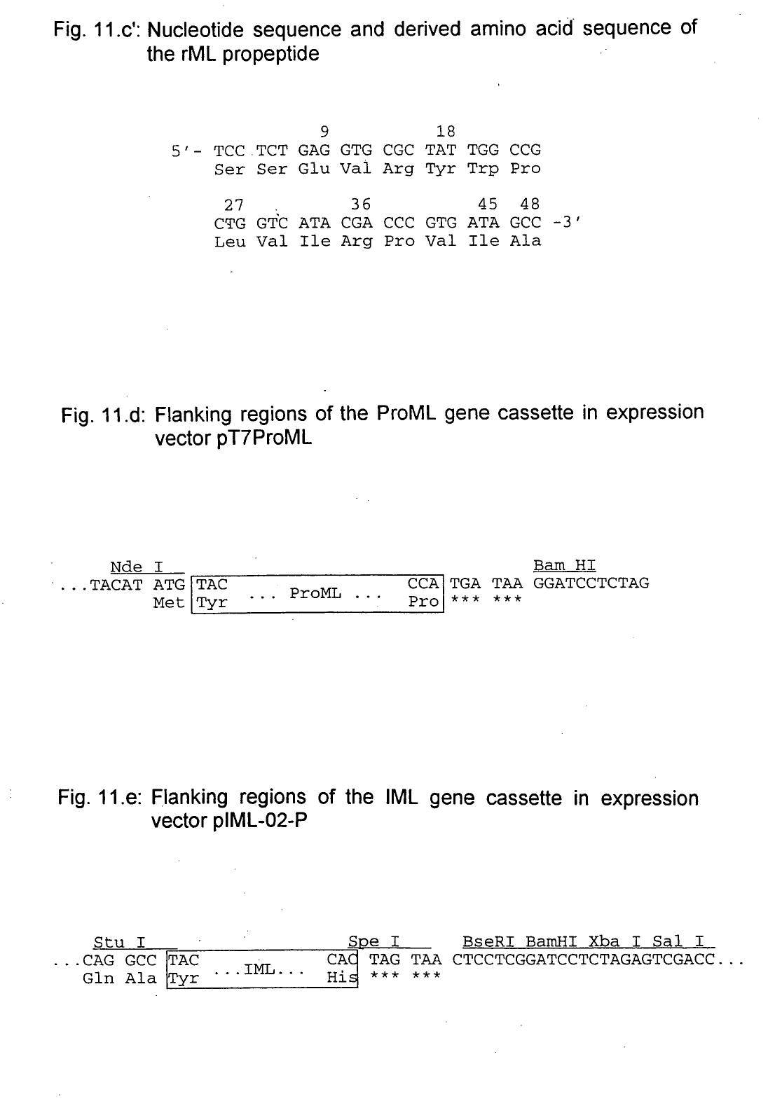

In a preferred embodiment the invention relates to a nucleic acid molecule, wherein
(a) the mistletoe lectin A chain is encoded by a nucleic acid molecule selected from the group consisting of:
   (i) nucleic acid molecules which comprise a nucleotide sequence encoding the amino acid sequence indicated in FIG. 11.a or a fragment thereof;
   (ii) nucleic acid molecules which comprise the nucleotide sequence indicated in FIG. 11.a or a fragment thereof; and
   (iii) nucleic acid molecules which hybridize to a nucleic acid molecule from (i) or (ii); and
   (iv) nucleic acid molecules which are degenerate to the nucleic acid molecules mentioned in (iii); and/or

(b) the mistletoe lectin propeptide is encoded by a nucleic acid molecule selected from the group consisting of:
(i) nucleic acid molecules which comprise a nucleotide sequence encoding the amino acid sequence indicated in FIG. 11.c or a fragment thereof;
(ii) nucleic acid molecules comprising the nucleotide sequence indicated in FIG. 11.c or a fragment thereof;
(iii) nucleic acid molecules which hybridize to any nucleic acid molecule from (i) or (ii); and
(iv) nucleic acid molecules which are degenerate to the nucleic acid molecules mentioned in (iii).

Hybridization in the context of the invention means hybridization under conventional hybridization conditions. Preferably, hybridization is carried out under stringent conditions. Such conditions are described, e.g., in Sambrook et al., “Molecular Cloning, A Laboratory Handbook”, CSH Press, Cold Spring Harbor, 1989, or in Hames and Higgins “Nucleic acid hybridisation”, IRL Press, Oxford, 1985. Such conditions are, for example, achieved with a hybridization buffer containing 0.1×SSC and 0.1% SDS. The hybridization and, if applicable, subsequent washing steps (washing buffer optionally contains also 0.1×SSC and 0.1% SDS) are carried out at about 65° C.

In another embodiment the invention relates to a nucleic acid molecule, wherein
(a) the effector module possesses the biological activity of the mistletoe lectin A chain and comprises an allele or derivative of the above-mentioned mistletoe lectin A chain by amino acid deletion, substitution, insertion, addition and/or exchanges; and/or
(b) the processing module is proteolytically cleavable and comprises an allele or derivative of the above-mentioned mistletoe lectin propeptide by amino acid deletion, substitution, insertion, addition and/or exchanges.

The above-mentioned alleles and derivatives can be naturally occurring or artificial, e.g., alleles and derivatives generated by recombinant DNA techniques. They include molecules which differ from the above-mentioned nucleic acid molecules by degeneration of the genetic code. It is a matter of fact that posttranslational or modifications carried out only after production of the above-mentioned changes of the above-mentioned effector modules and/or processing modules still are subsumed under the term derivatives as long as these derivatives have the same or similar activity and/or function as the above-mentioned effector modules and/or processing modules.

In another preferred embodiment the invention relates to a nucleic acid molecule, wherein the fusion protein furthermore comprises the following components: (d) a modulator module which is covalently linked to the processing module, the effector module and/or the targeting module and which modulates the intracellular toxic effect of the effector module.

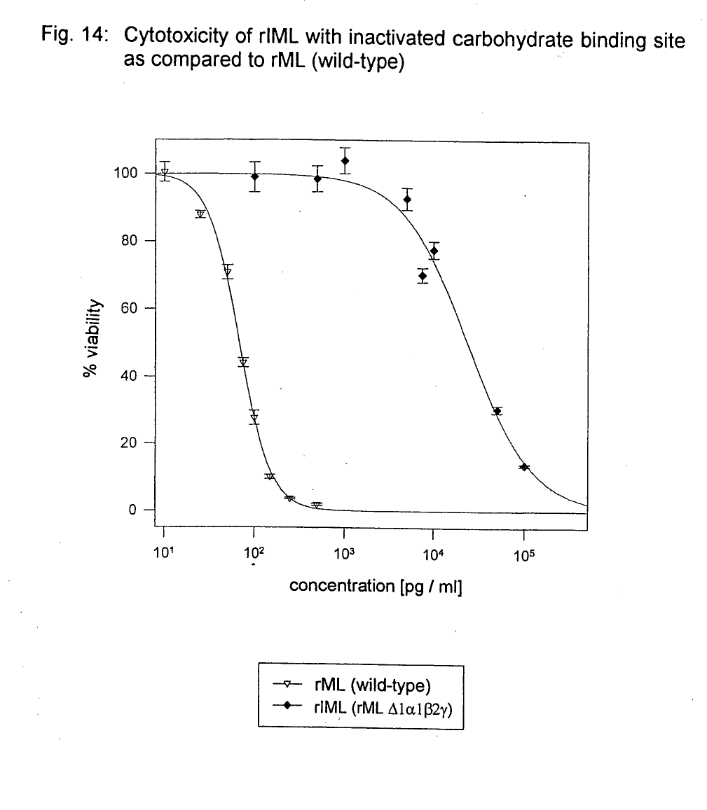

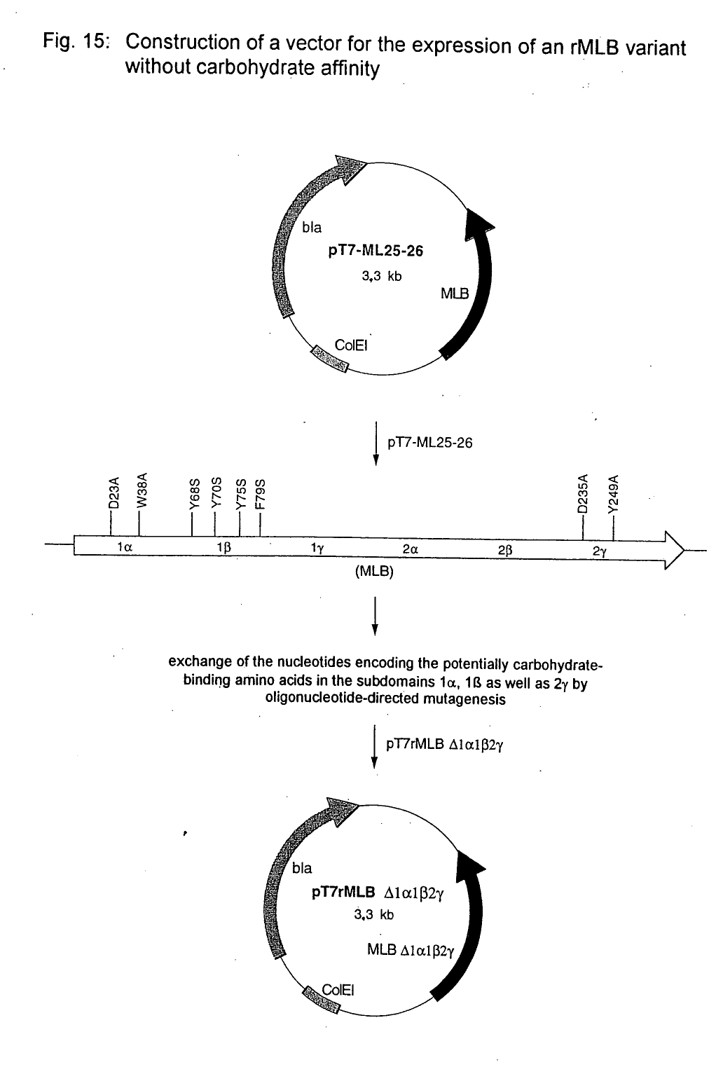

In the context of the present invention, all polypeptide sequences are understood as “modulator module” which are capable of intracellularly modulating the cytotoxic effect of an effector module and which are linked to at least a further module of the fusion protein according to the invention on the genetic level preferably by a processing module linking both modules. Examples of suitable modulator modules are components which assist in membrane translocation or those that participate in intracellular transport mechanisms. The desired modulation preferably resides in enhancing the cell-type specific effectiveness or in avoiding unspecific toxicity. For rMLA it was found that these requirements are met by the recombinant B domain of the mistletoe lectin (rMLB), which effects an increase in toxicity of the effector module by actively supporting its translocation from the endoplasmic reticulum to the cytoplasm of the cell. In the past it was already shown that the cytotoxic effect of this class of substances may be increased by several orders by using type II RIPs instead of type I RIPs for producing, e.g., antitumoral agents, but that the therapeutic effect of these preparations which was hoped for could not be achieved in the last analysis because of the very grave side-effects. A possible way out of this dead-end is shown by attempts at inactivating by chemical derivatization the sugar-binding moieties of the ricin B chain after coupling to the antibody—so-called “blocked ricin” (Shah et al., 1993)—which, however, did not at all solve the problem because the substances still carried severe side-effects. In a particularly preferred embodiment of the present invention for the first time the attempt is made to exchange by using molecular-biological methods the amino acids responsible for sugar binding for amino acids that are biologically not functional (functionally inert) in this respect. For ricin which is similar to mistletoe lectin two sugar binding moieties have been known from the art for some time in the 1α and 2γ sub-domain of the B chain (Rutenber et al., 1987; Vitetta et al., 1990; Swimmer et al., 1992; Lehar et al., 1994). The tests carried out in the present invention on the basis of these findings to inactivate the carbohydrate affinity of the recombinant mistletoe lectin have shown that the sugar binding moieties described for ricin can also be found in mistletoe lectin. Surprisingly, however, it turned out that the exchanges of the analogous amino acids described for ricin do not switch off the sugar binding moiety of the mistletoe lectin but can only attenuate it by factor 5. A subsequent more detailed analysis of the crystal structure of ricin B for the presence of other cryptic sugar binding moieties by computer-aided calculations of the field of force has indicated that there may be a third sugar binding moiety—both for lactose and for N-acetyl-neuraminic acid—in the 1β-sub-domain. Literature reported a third sugar binding moiety for ricin B—there, too, in the 1β domain—with the participation of a single amino acid (Frankel et al., 1996), which additionally corroborates the above assumption. After substitution of the four amino acids which on the basis of the calculations are presumed to be involved in carbohydrate binding of the 1β domain of the recombinant mistletoe lectin, in addition to the exchanges in the 1α and 2γ domain (Example 7, FIG. 15), in fact an almost complete loss of ability of the B chain variant “rMLB Δ1α1β2γ” to bind to a lactosyl-agarose affinity matrix could surprisingly be observed. Furthermore, rMLB Δ1α1β2γ (rIMLB) did not only show the same folding competence as the wild-type sequence but it was still capable of covalently associating with the recombinant mistletoe lectin A chain (Example 8.c). FIG. 13 shows a Western blot analysis of the in vitro association of rMLB Δ1α1β2γ with rMLA using immunochemical detection with monoclonal antibodies against both single chains in the size of the expected molecular weight of the holotoxin of about 60 kDa. The cytotoxicity of the non-carbohydrate binding holo-toxin (rIML) so obtained vis-à-vis the human lymphatic cell line MOLT-4 shows 50% viability at an rIML concentration of 25 ng/ml. This corresponds—vis-à-vis 70 pg/ml when rML is used—to an attenuation of the unspecific in vitro toxicity by factor 350 (Example 9, FIG. 14).

The availability of such a modified modulator module (rIMLB) for the first time makes it possible to recombinantly produce anti-immune cell toxins for which there are chances that the fatal side-effects of the substances so far available on the basis of the natural type II RIPs may be reduced to a tolerable extent by using rIMLB. In order to guarantee a targeting module-mediated specificity the carbohydrate binding can be minimized in the case of rMLB by targeted amino acid exchange, for example exchanging D23 for A, W38 for A, D235 for A, Y249 for A, Y68 for S, Y70 for S, Y75 for S, F79 for S (the nomenclature refers to the amino acid sequence of the rMLB according to FIG. 11b with D1 as N-terminal amino acid).

In another preferred embodiment the invention relates to a nucleic acid molecule, wherein the modulator module is encoded by a nucleic acid molecule selected from the group consisting of:
(i) nucleic acid molecules which comprise a nucleotide sequence encoding the amino acid sequence indicated in FIG. 11.b or a fragment thereof;
(ii) nucleic acid molecules which comprise the nucleotide sequence indicated in FIG. 11.b or a fragment thereof;
(iii) nucleic acid molecules which hybridize to a nucleic acid molecule from (i) or (ii); and
(iv) nucleic acid molecules which are degenerate to the nucleic acid molecules mentioned in (iii).

In another preferred embodiment the invention relates to a nucleic acid molecule, wherein the modulator module possesses the above-mentioned modulating activity and comprises an allele or derivative of the above-mentioned mistletoe lectin B chain by amino acid deletion, substitution, insertion, addition and/or exchanges.

It has already been discussed above how the terms “hybridization”, “alleles” and “derivatives” are to be understood in the context of the present invention. These terms have to be applied mutatis mutandis for the embodiments discussed herein.

As further modulator modules in the context of the present invention short peptide fragments such as the peptides having the amino acid sequences KDEL (SEQ ID NO: 35) or HDEL (SEQ ID NO: 36) are used. These peptides are signal peptides which mediate the active retrograde transport of proteins in direction of the endoplasmic reticulum, which can be used to increase the toxicity of the effector modules taken up (Wales et al., 1993). In the context of the invention, polypeptide sequences which keep the catalytic activity of an effector module outside a cell neutral are likewise to be classified as modulator module. An example of these sequences is the propeptide of the mistletoe lectin which inactivates the catalytic activity of rMLA and releases the catalytic activity of rMLA only during intracellular processing in prelysosomal cell compartments, offering the advantage of a drastically reduced unspecific toxicity of fusion proteins circulating in the blood.

The modulation of the toxicity by a modulator module is very important. For example, it may be desirable to reduce in target cells the toxicity of an effector module in order to achieve more advantageous interferences with the target cell. For example, it may be desired to kill target cells slowly so as to avoid that potentially detrimental cellular components are released into the organism. Detrimental reactions like immediate-type hypersensitivities or anaphylactic shocks can be avoided. It is also possible to induce cellular programmed processes such as apoptosis by modulating the toxic effects. Apoptosis is a natural mechanism of clonal selection and thus a comparatively gentle method for the surrounding tissue and the entire organism of specifically eliminating pathological cells.

In context with this embodiment it was found according to the invention that rMLB can modulate the toxicity of rMLA, which offers the possibility of specifically influencing the toxicity of the fusion proteins according to the invention. This finding is of utmost importance for the field of medicine, since for the first time ever it is possible to vary the effect of one and the same immunotoxin in one and the same cell by choosing a suitable modulator. The person skilled in the art of course starts from the assumption that the modulating effect of the rMLB chain also has an effect on other toxins such as those of the RIP I- or RIP II-type. Based on the knowledge of the modulating effect of the rMLB chain the person skilled in the art is readily capable of testing the modulating effect of other sugar-binding molecules, e.g., of those molecules that naturally occur in type II-RIPs. The property of the mistletoe lectin B chain to have a modulating effect on the uptake and activation of effector molecules extending beyond the binding of sugar moieties raises expectations that at least other type II RIP B chains of plant origin have a similar property profile. Such modulators can also be advantageously used in the context of the invention. Such modulators are also comprised by the present invention.

In another preferred embodiment of the invention the nucleic acid molecule for the fusion protein furthermore displays the following component:
(e) an affinity module which is covalently linked to the effector module, the processing module, the targeting module and/or the modulator module.

Components of the fusion proteins according to the invention are referred to as affinity modules which do not have a therapeutic effect but offer the possibility of purifying the fusion proteins according to the invention, by, e.g., methods of affinity chromatography. Other methods such as ion exchange, gel permeation or hydrophobic interaction chromatography, with which the fusion proteins can be purified, are well-known to the person skilled in the art. When affinity modules are used it is possible to obtain preferably homogeneous or essentially homogeneous substances using methods of affinity chromatography. Ideally, the affinity modules are short peptide fragments such as a hexahistidine sequence with affinity to sepharose chelate complexes which are preferably fused to the sequence periphery (FIG. 10.a-10.g). This embodiment of the invention allows a quick and unproblematic purification of the fusion protein according to the invention.

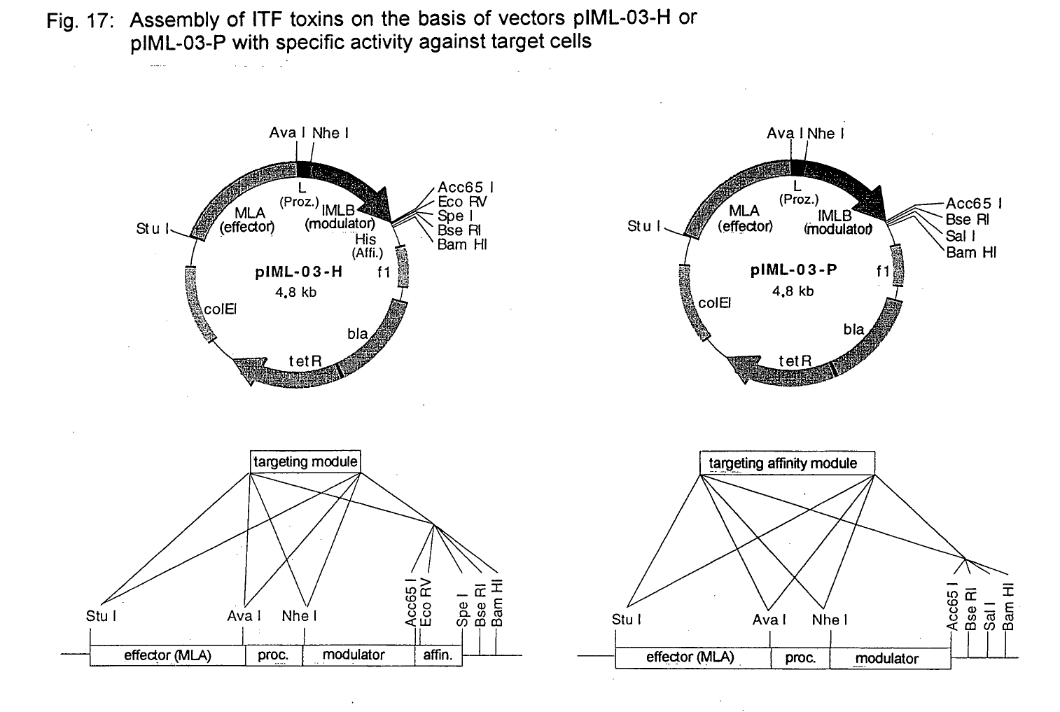

Due to the recombinant production of the fusion protein the modules mentioned in the above-mentioned embodiments can be arranged in the desired sequence by freely combining the corresponding nucleic acid sequences. On the basis of his expert knowledge the person skilled in the art is capable of producing corresponding recombinant nucleic acid molecules, for example by introducing suitable restriction cleavage sites. A selection of possible combinations or arrangements is shown in FIG. 10.a-10.g. The periplasmic cell compartment of E. Coli most closely meets the requirements of a disulfide bond-containing protein on the microenvironment required for the formation of a functional tertiary structure. Starting therefrom, as described in detail in Example 10, a periplasmic modular expression system was constructed which allows the realization of any arrangements required of the modules in the ITF expression vectors (FIG. 17).

In another preferred embodiment of the nucleic acid molecule according to the invention the processing module is of plant origin and comprises or preferably contains the sequence SSSEVRYWPLVIRPVIA (SEQ ID NO: 37) of the ML propeptide. Other propeptides, too, which are encoded by RIP genes in plant genomes are suitable as or contain processing modules. The person skilled in the art is capable on the basis of his expert knowledge and the teaching provided by the invention of selecting or constructing such processing modules. In still further embodiments peptides which exhibit the general amino acid sequence S4-S3-S2-S1-/S1′ can be used as proteolytic cleavage sites for the optionally N or C terminal fusion to an effector module. S2 preferably means the amino acid residues phenylalanine, tyrosine, valine or leucine and represents a recognition site for proteases of the cathepsin family. Another advantageous cleavage site is present if S1 is arginine or lysine, which generates a recognition site for proteases of the trypsin family. The risk of an unspecific effect of a fusion protein according to the invention on healthy cells can be reduced by using recognition sites for cell-type specific proteases such as the elastase of granulocytes, with S1 preferably being alanine or serine. S3 and S4 can be any amino acid residues except proline.

In another preferred embodiment of the nucleic acid molecule according to the invention the targeting module specifically recognizes a cell of the immune system, a tumor cell or a cell of the nervous system.

The main emphasis of the present research projects is in the field of the set of receptors of immune cells, which results in a quickly growing number of known receptors as well as their ligands. Due to the modular nature of the fusion proteins according to the invention new findings in this field can be converted to the production of therapeutically useful substances more quickly than before. This aspect is gaining particular importance in the development of preparations which are individualized for the patient. Promising possible uses of such modular fusion proteins are in the treatment of dysfunctions of the nervous and of the immune system. These cells are cells that mainly circulate in the blood or lymphatic system which are physically well accessible to the fusion proteins according to the invention. The problems of poor tumor penetration by immunotoxins therefore do not occur. Also, particularly for cells of the immune system apoptosis is a natural mechanism of the clonal expansion control so that the use of, e.g., rMLA as effector module advantageously uses the natural susceptibility of the immune cells for apoptosis (cf. also Bussing et al., 1996). Furthermore, the advantages of a modular system typically lend themselves for the treatment of allergies, since a broad range of various patient-specific targeting modules is required in this field. For example, in the case of allergies of the immediate type a T_{H}2 cell induced B cell class switch to the allergenic IgE production takes place in contrast to the T_{H}1 cell mediated IgG response. One therapeutical approach using the fusion proteins according to the invention is to use allergenic peptides which normally present MHCII as targeting modules and thus to selectively eliminate the responsive T_{H}2 cells from the patient's body. The same principle allows a therapy of autoimmune diseases. The therapeutical approaches currently used for MS as an example of autoimmune diseases include diverse interferences with the regulation of the immune system (Hohlfeld, 1997). The causal treatment of autoimmune diseases concentrates on the depletion of the respective autoantigen-specific T cells. A presently favored approach is based on the expression of a specific TCR subtype, for example, for MS the activity of the MBP-reactive T cells could be modulated by vaccination with the Vβ35.2 peptide (Vandenbark et al., 1996). The principle underlying this method is mainly based on a shift of the cytokine response from T_{H}1 to T_{H}2, i.e., from proinflammatory to inhibitory cytokines. In the final analysis, a systemic effect is achieved.

In the case of the demyelinating neuropathy (Guillain-Barré syndrome, neuritis) the autoantigen is the myelin of the peripheral nervous system (P2). In the animal model of the neuritis EAN the aa region 53-78 could be identified as neuritogenic peptide. EAN can be induced either actively by the neuritogen P2 directly or by adoptive transfer of neuritogenic T cells which were isolated from diseased rats.

The recombinant P2 peptide was already successfully used for alleviating EAN in rats, while making use of the apoptosis-inducing effect of P2 (dosage 100 μg daily i.v.; Weishaupt et al., 1997).

A prerequisite for the alleviation of an incident in a patient is that a correspondingly high, apoptosis-inducing concentration of the antigen reaches the autoreactive T cells in the periphery or at the site of the autoimmune response. When small amounts of antigen are bound to T cells they naturally proliferate. The coupling of the toxin to the specific recognition sequence of the neuritogenic T cells can thus mediate a prompt T cell elimination, without risking an adverse stimulatory effect. The trigger for, e.g., Multiple Sclerosis is the production and proliferation of autoreactive T-lymphocytes (Olive, 1995) which recognize a degradation product of the “myelin-basic-protein”—in most cases the sequence VHFFKNIVTPRTP (SEQ ID NO: 38). The result is that the nerve cells of the patient are being attacked by the body's own immune system. Here, too, the use of pathogenetic peptides as targeting modules is the key to the application of a therapy based on the invention. A similar disease is Myasthenia Gravis, where there is an autoimmune response to acetylcholine receptors. Further potential fields of application are the treatment of diverse leukemias or neoplasias.

Thus, in a particularly preferred embodiment of the invention the target cell is a cell of the immune system. It may be a cell of the unspecific immune system or a cell of the specific immune system. In the latter case, it may be B cells or T cells, particularly T_{H}2 cells. Also, degenerate cells of the immune system can be target cells. Also cells, particularly degenerate cells of the nervous systems, for example nerve cells, may be target cells for the selection of suitable targeting modules.

In another preferred embodiment of the nucleic acid molecule according to the invention, the affinity module is a histidine sequence, thioredoxin, a maltose-binding protein, or GFP (green fluorescent protein). Additionally, the affinity module may be STREP-TAG®, available from IBA GmbH, Gottingen, Germany, a peptide having highly selective binding affinity for engineered streptavidin; the FLAG peptide (DYKKDDDK[SEQ ID NO: 39]), available as FLAG-TAG® from Strategene, Corp., La Jolla, Calif., U.S.A; or T7-TAG®, a T7 peptide that is an 11 amino acid gene leader peptide, available commercially from CN Biosciences Corp., Darmstadt, Germany. The affinity module is a peptide sequence which is characterized by a ligand binding specificity or by the presence of suitable epitopes which allows a selective purification preferably by affinity chromatography methods, e.g., by way of immobilized ligands or immobilized antibodies. Such affinity modules always have the property of binding ligands very specifically and with high binding constants, which in turn are preferably coupled as ligands to chromatographic matrices. In this way, highly purified fusion proteins from lysates or cell supernatants can be produced using processes with only few steps.

Another preferred embodiment of the present invention relates to a nucleic acid molecule, wherein the modulator module comprises the mistletoe lectin B chain or a fragment or derivative thereof or the peptides KDEL (SEQ ID NO: 35) or HDEL (SEQ ID NO: 36).

In this embodiment, for example, the rMLB-sequences are replaced by fragments or derivatives of rMLB. As already discussed above in context with the use of the rMLA chain, the person skilled in the art on the basis of his expert knowledge is capable of recombinantly providing nucleic acids which encode such fusion proteins. With respect to a test with which the modulator function of the fragments or derivatives can be detected, reference is made to the examples below.

In a particularly preferred embodiment of the nucleic acid molecule according to the invention the mistletoe lectin B chain exhibits an exchange in amino acid positions 23, 38, 68, 70, 75, 79, 235 or 249 or a combination of such exchanges. Particularly preferred is the embodiment, whereby the exchanges are in position D23 for A, W38 for A, D235 for A, Y249 for A, Y68 for S, Y70 for S, Y75 for S, F79 for S (the nomenclature relates to the amino acid sequence of the rMLB according to FIG. 11b with D1 as N-terminal amino acid).

This embodiment is particularly preferred because the amino acid residues in the positions mentioned participate in the formation of sugar binding moieties which can bind the sugars or glycoproteins or glycolipids on cell surfaces. An elimination of sugar binding sites has the effect that an unspecific, sugar-mediated attachment to undesired cells is avoided. The frequency with which the fusion protein according to the invention actually reaches the site of intended effect is thus significantly increased.

In another preferred embodiment of the present invention the nucleic acid molecule is DNA.

In another preferred embodiment of the present invention the nucleic acid molecule is RNA.

The invention furthermore relates to a vector which contains the nucleic acid molecule according to the invention.

The construction of suitable vectors for the propagation and preferably the expression of the nucleic acid according to the invention is known to the person skilled in the art. As far as the vector is used for producing the fusion protein the skilled person will want to achieve an as high as possible yield of fusion protein and will therefore introduce a strong promoter into the vector. It may, however, be advantageous, for example if the vector is a component of a medicament, that the expression of the nucleic acids is switched on only in the target cell. In this case, the person skilled in the art will choose an inducible expression system. In the context of the present invention, the vector may contain more than one nucleic acid according to the invention.

For the expression or propagation of the vector a suitable host is required. Thus, the invention furthermore relates to a host which is transformed with the vector according to the invention or which contains a nucleic acid molecule according to the invention. The invention comprises also those hosts which contain several vectors and/or nucleic acid molecules according to the invention.

Transformation methods have been described in the art for the various cell types and host organisms and can be chosen by the skilled person depending on suitable aspects.

According to the invention, the following prokaryotic hosts are particularly preferred: E. coli, Bacillus subtilis or Streptomyces coelicolor and the following eukaryotic hosts: Saccharomyces sp., Aspergillus sp., Spodoptera sp. or Pichia pastoris. For eukaryotic expression systems it is particularly advantageous to use modulator modules since a damage of the host by the expression product can be avoided when a modulator module is used.

The invention furthermore relates to a fusion protein which is encoded by a nucleic acid molecule according to the invention or produced by a host according to the invention.

The advantages and possible uses of the fusion protein according to the invention have already been discussed in context with the various embodiments of the nucleic acid molecule according to the invention to which reference is herewith made.

Furthermore, the invention relates to a process for producing the fusion protein according to the invention, whereby a host according to the invention is grown under suitable conditions and the fusion protein is isolated.

Preferably, the process according to the invention is a microbiological, fermentative process that is carried out under conventional conditions. The fusion protein generated may be isolated from the supernatant or from the host after it has been broken up. The latter embodiment includes denaturing and renaturing the fusion protein as far as it is produced, for example in bacteria, in the form of inclusion bodies.

The implications for the pharmaceutical sector and the fundamental importance of the invention for medicine has already been discussed above. Accordingly, the invention also relates to a medicament which contains a fusion protein according to the invention and a pharmaceutically acceptable carrier.

So far, the attempts described for the production of immunotoxins using the A domain of the mistletoe lectin had to use the route of biochemical coupling, e.g., with SPDP (Paprocka et al., 1992). In two respective studies (Tonevitsky et al., 1991, 1996) the effectiveness of the nMLA immunotoxins obtained was compared with the corresponding ricin A immunotoxins, wherein the nMLA immunotoxins proved to have an effectiveness that was 15-80 times higher than that of the immunotoxins on the basis of ricin A. The possibility of taking recourse to recombinantly produced mistletoe lectin components, which was not part of the prior art, facilitates the production of the medicament according to the invention.

The form and dosage of administration of the medicament according to the invention is to be chosen by the attending physician who is particularly familiar with the condition of the patient. Other factors which may influence form and dosage of administration are age, sex, body surface area and weight of the patient as well as the route of administration. Pharmaceutically acceptable carriers are known in the art and comprise phosphate-buffered saline solutions, water, emulsions such as oil/water emulsions, etc. Pharmaceutical compositions comprising such carriers can be formulated according to conventional methods. The medicament may be administered systemically or locally and will usually be administered parenterally. Usual routes of administration are, e.g., intraperitoneally, intravenously, subcutaneously, intramuscularly, topically or intradermally. Intravenous administration is preferred. Preferred dosages for the intravenous administration are in the range of 1 ng active substance per kg body weight up to 500 μg/kg. For ex vivo applications dosages in the range of 1 pg/ml to 500 ng/ml are preferred. Preferably, these dosages are administered daily. As far as the treatment requires continued infusion, the dosages also are within the above ranges.

Furthermore, the invention relates to a medicament which contains
(a) a fusion protein which is encoded by a nucleic acid molecule according to the invention, wherein the fusion protein comprises an effector, processing, targeting and optionally an affinity module or a vector which contains the nucleic acid molecule; and
(b) a modulator module which is covalently linked to a processing module and/or an effector module which modulates the intracellular toxic effect of the effector module or a vector which contains a nucleic acid encoding the modulator module.

The modulator module may be covalently linked in the medicament according to the invention to the other modules and thus be encoded by the same vector as those modules or it may occur as a separate unit and is encoded, e.g., by a second vector, preferably, however, it is encoded together with the other modules by sequences present in a single vector.

In the embodiment, in which the medicament contains the above-mentioned polypeptides the latter are preferably produced as covalently linked fusion protein before the medicament is formulated, thereby particularly ensuring that the polypeptide complex which exhibits both the effector, processing and targeting module as well as the modulator module is incorporated into one and the same target cell. If the medicament contains the vector(s) according to the invention, usually 10⁶ to 10²² copies per vector are applied according to the above-mentioned schemes of administration. The vectors according to the invention may also be used in gene therapy. Methods for a use of the vectors in gene therapy are likewise known in the prior art.

The embodiment according to which the medicament contains the vectors is particularly advantageous if no immediate effect of the toxin is desired. This may, for example, be the case, if the medicament is administered as accompanying therapy. In this embodiment the target cell specificity is achieved by using a suitable vector, for example a retroviral vector. A number of retroviral vectors are known from the state of the art which are specific of, e.g., T cells. Expression of the nucleic acids may, for example, be achieved via temperature-sensitive promoters. In practice, for example, the patient can be exposed for a suitable period to a heat source by which expression of the nucleic acids is switched on and the toxin develops the desired effect in the target cell.

In a preferred embodiment of the medicament according to the invention discussed above, the modulator is or comprises the mistletoe lectin B chain or a fragment or derivative thereof.

For the above reasons it is therefore preferred that the mistletoe lectin B chain exhibits an exchange in amino acid positions 23, 38, 68, 70, 75, 79, 235 or 249 (the nomenclature relates to the amino acid sequence of the rMLB according to FIG. 11.b with D1 as N-terminal amino acid) or a combination of such exchanges, the exchange in position 23 preferably being an exchange of D23 for A, in position 38 preferably W38 for A, in position 235 preferably D235 for A, in position 249 preferably Y249 for A, in position 68 preferably Y68 for S, in position 70 preferably Y70 for S, in position 75 preferably Y75 for S and in position 79 preferably F79 for S. It is particularly preferred, like in the embodiments discussed hereinbelow which refer to these exchanges, that the chain contains at least two, preferably at least three, four, five, six, seven and most preferably 8 exchanges.

The invention furthermore relates to a kit containing
(a) a vector which contains a nucleic acid molecule according to the invention; and/or
(ba) a vector which contains a nucleic acid molecule according to the invention, wherein the nucleic acid molecule encodes an effector, processing, targeting and optionally an affinity module; and
(bb) a vector which contains a nucleic acid molecule encoding a modulator which modulates the intracellular toxic effect of the effector module.

In particular, the kit according to the invention allows to examine the efficiency of the various modules in various/on various target cells in vitro. Exemplarily of the in vivo situation, e.g., neoplastically transformed cells are cultivated in vitro and transfected with the vectors according to embodiment (a) or according to embodiments (ba) and (bb). The effect of expression of the various modules on the viability of the transfected cells can be observed, for example, under the microscope. Thus, the kit according to the invention provides valuable results for the development of medicaments, for example, for tumor therapy.

In a preferred embodiment, the modulator in the kit according to the invention is the mistletoe lectin B chain or a fragment or derivative thereof.

It is particularly preferred that the mistletoe lectin B chain exhibits an exchange in amino acid positions 23, 38, 68, 70, 75, 79, 235 or 249 or a combination of such exchanges, the exchange in position 23 preferably being an exchange of D23 for A, in position 38 preferably W38 for A, in position 235 preferably D235 for A, in position 249 preferably Y249 for A, in position 68 preferably Y68 for S, in position 70 preferably Y70 for S, in position 75 preferably Y75 for S and in position 79 preferably F79 for S.

The invention furthermore relates to the use of the mistletoe lectin B chain or a fragment or derivative thereof for modulating the effectiveness of an intracellularly active toxin.

As already discussed above, the present invention for the first time ever shows that the sugar-binding component of a type II-RIP is capable of intracellularly modulating and particularly of increasing the cytotoxic effect of a toxin. According to the invention it is expected that, e.g., the mistletoe lectin B chain does not only modulate the toxicity of the mistletoe lectin A chain but also that of other toxins, particularly of those of type I or type II-RIP. The teaching of the present invention allows the person skilled in the art to easily determine whether the modulator actually modifies the toxicity of a toxin of interest or not. In this regard, the use according to the invention comprises the use of all intracellular toxins and not only the mistletoe lectin A chain.

According to the invention a use is preferred wherein the toxin intracellularly is a cleavage product of a fusion protein which exhibits the following components:
(a) an effector module which comprises the toxin;
(b) a processing module which is covalently linked to the effector module and which exhibits a recognition sequence for a protease; and
(c) a targeting module which is covalently linked to the processing module and which specifically binds to the surface of a cell, thereby mediating the internalization of the fusion protein into the cell; and optionally
(d) an affinity module which is covalently linked to the effector module, the processing module, the targeting module and/or the modulator module.
   This preferred embodiment makes additional use of the modular concept according to the invention which has been described earlier. In this regard, this embodiment offers particular practical advantages for the development of medicaments.

Particularly preferred is a use, according to which the mistletoe lectin B chain exhibits an exchange in amino acid positions 23, 38, 68, 70, 75, 79, 235 or 249 or a combination of such exchanges and wherein the exchange in position 23 is preferably an exchange of D23 for A, in position 38 preferably W38 for A, in position 235 preferably D235 for A, in position 249 preferably Y249 for A, in position 68 preferably Y68 for S, in position 70 preferably Y70 for S, in position 75 preferably Y75 for S and in position 79 preferably F79 for S.

Furthermore preferred is a use according to which the toxin is the A chain of type II RIPs (mistletoe lectin, ricin, abrin, ebulin, modeccin and volkensin) or of type I RIPs (saporin, gelonin, agrostin, asparin, bryodin, colocin, crotin, curzin, dianthin, luffin, trichosanthin and trichokirin), or an intracellularly toxic fragment or derivative thereof.

The invention also relates to a method for testing in vitro a prospective modulator by carrying out the following steps:
(a) transfecting a target cell with a vector which contains a nucleic acid molecule encoding an effector, processing, targeting and optionally affinity module;
(b) transfecting a target cell with a vector which contains a nucleic acid encoding a prospective modulator;
(c) expressing the nucleic acids in the target cell; and
(d) measuring the modulating activity of the prospective modulator on the toxicity of the toxin.

The process according to the invention can be used to test a multitude of prospective modulators which may be of different origin. Preferably, the modulators are of plant origin. In a preferred embodiment, the process can be used to test the influence of modifications on a modulator. For example, a modulator can be modified by recombinant techniques such that it exhibits an additional domain which is not present in a natural state and which fulfills a desired biological function. The process according to the invention can be used to test whether and in how far this modification influences the modulating properties of the modulator. As a matter of course, other modifications to the modulator commonly known to the person skilled in the art can be tested with this process. The skilled person can choose suitable target cells in accordance with his experimental objectives.

It is possible for the person skilled in the art to stably or transiently introduce a nucleic acid molecule encoding an effector, processing, targeting and optionally affinity module into a desired target cell. Accordingly, the invention furthermore relates to a process for testing in vitro a prospective modulator by carrying out the following steps:
(a) transfecting a target cell which contains a nucleic acid molecule encoding an effector, processing, targeting and optionally affinity module with a vector which contains a nucleic acid encoding a prospective modulator;
(b) expressing the nucleic acids in the target cell; and
(c) measuring the modulating activity of the prospective modulator on the toxicity of the toxin.
   Finally, the invention relates to a process for preparing a modulators, by carrying out the above-described in vitro test methods and additionally the following step: (e) or (d) isolating the modulator.
   The isolation may preferably be carried out according to standard techniques.

Before the invention is explained by way of the examples, general aspects are presented of how the invention may technically be put into practice on the basis of the general expert knowledge:

The modular nature of effector module (E), modulator module (M), targeting module (T), processing module (P) and affinity module (A) is usually brought about by introducing suitable restriction sites at the N and C terminus of the corresponding nucleic acid molecules or genes. The nucleic acid sequence of the effector module, in the embodiment of rMLA discussed herein, contains a recognition sequence of the restriction endonuclease NdeI at the N terminus, which allows for the N-terminal fusion of the effector module to processing modules (Example 1). C-terminal fusions are facilitated by, e.g., an AvaI restriction site (FIG. 11.a). In the sequence encoding the modulator module (preferably rMLB) for example the N-terminal restriction site StuI or BspLU11 I and the C-terminal restriction site EcoRV may be used for gene fusion with other modules (FIG. 11.b). Processing modules which can be obtained from, e.g., the recombinant propeptide of the mistletoe lectin (FIG. 11.c), may be adapted to the respective required restriction sites and the respective target cell specific protease profile in form of chemically synthetized gene cassettes due to their short sequence. The latter may even increase the selective effect of the fusion proteins according to the invention.

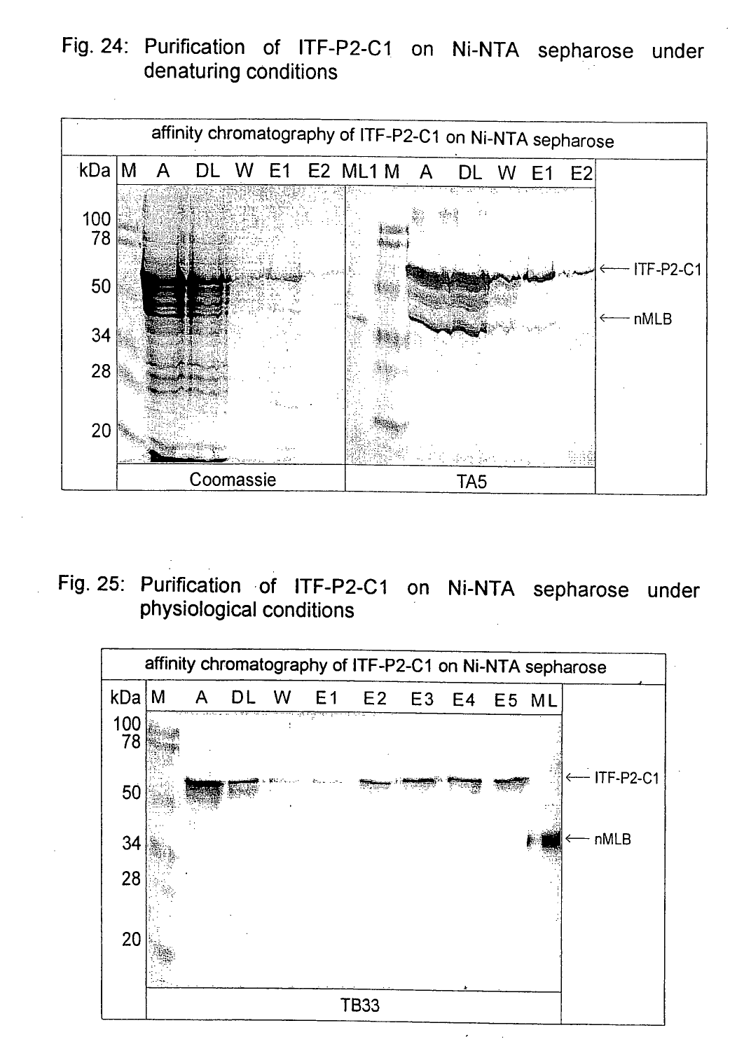

The provision of the fusion proteins in highly purified form is preferably achieved by one or several chromatographic steps, preferably by affinity chromatography which permits an enrichment of the fusion proteins for example using the affinity modules. Furthermore, a selection for a functional targeting module may allow further purification. The purification steps may be carried out in any order whatever. Example 3 shows the use of a two-step purification method without using an affinity module. In the first step, the fusion protein according to the invention is purified via its targeting module mediated heparin affinity and in the second step it is further purified via an immobilized antibody which exhibits affinity to the effector module. The most effective method for enriching proteins from cell extracts is affinity chromatography. Of particular advantage for the enrichment of ITFs is the use of the His-Tag as affinity module (hexahistidine sequence with affinity to nickel-NTA-sepharose), since even the presence of chaotrophic salts does not have a detrimental effect on the binding behavior. The use of the affinity modules “His-Tag” for producing ITFs is illustrated exemplarily for ITF-P2-C1 in native form in Example 12.b, in denatured form in Example 12.c. Thus, the proteins can be enriched and purified both in native (FIG. 25) as well as in denatured form (FIG. 24) so that the more advantageous method can be used depending on the specific behavior of the respective ITF variant. It is interesting to note that even when purification is carried out under denaturing conditions not only the exogenous protein is almost complete elimination but also the proteolytic degradation products (FIG. 24), which again emphasizes the advantageousness of this method. A process for producing soluble ITF starting from ITF-containing inclusion bodies that are dissolved in GuHCl is described in Example 12.c.

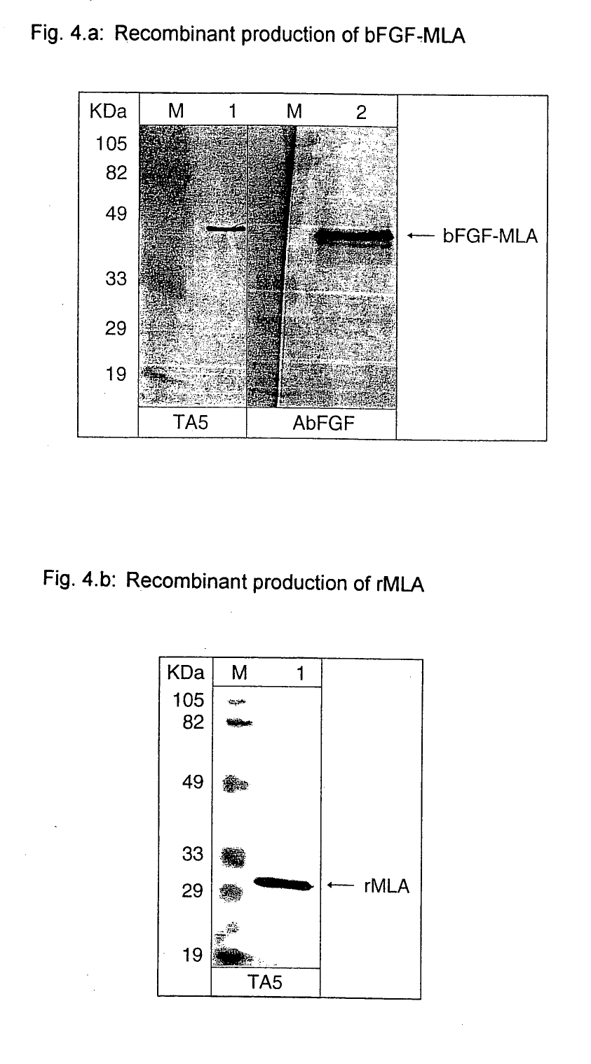

As an example of the fusion protein according to the invention of the TPE type (targeting, processing, effector module) the “basic fibroblast growth factor” (bFGF) was fused as targeting module to the N terminus of rMLA via a processing module. The processing module used is the protease-sensitive domain corresponding to a C-terminal sequence section of the bFGF. The domain is delimited from the N-terminal sequence section of bFGF by the presence of poorly defined elements of the secondary structure. Due to this property the protease recognition sequences in this section are recognizable for proteases of the target cells. The substance may be provided by heterologous expression of the fusion gene in E. coli in accordance with Example 3. FIG. 4.a shows the identity of the substance thereby obtained by immunological detection with the monoclonal anti-bFGF— and anti-nMLA antibodies in a Western blot analysis.

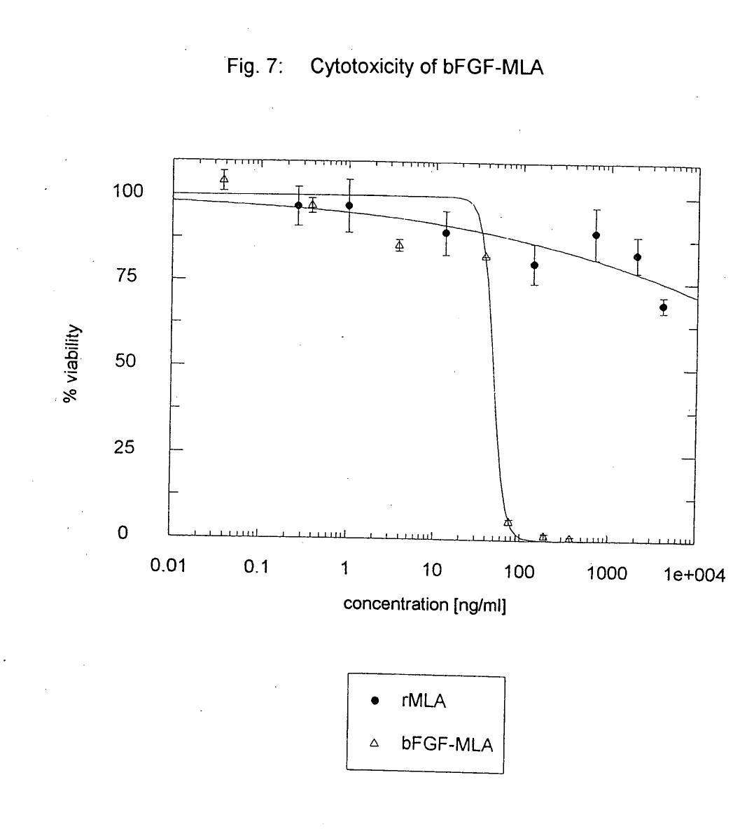

The functionality of such a bFGF-MLA fusion protein was shown vis-à-vis B16 cells according to Example 5. The advantage of using B16 cells is that it is known that they represent bFGF receptors on their cell surface to an increased extent. A comparison of the cell-killing effect of bFGF-rMLA (FIG. 4.a) with the effect of the effector module, in form of rMLA (FIG. 4.b) alone, impressively shows the realization of the concept according to the invention of using a targeting module. While rMLA does not have a toxic effect on the B16 cells in the concentration range of 200 pg/ml to 4 μg/ml examined, bFGF-MLA has a strong cytotoxic effect with a half-maximum viability (IC₅₀ value) of the B16 cells at a concentration of 48 ng/ml (FIG. 7.a). It was possible to show by way of the invention that the effector module rMLA, which is otherwise not effective can be selectively used to kill B16 cells by covalently linking it to a targeting module via a processing module.

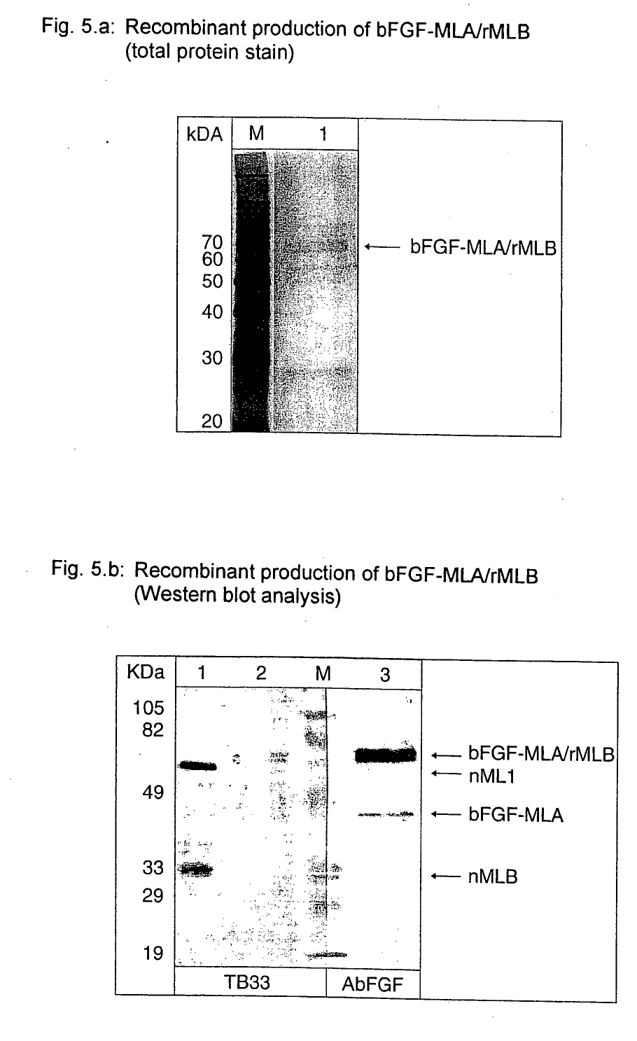

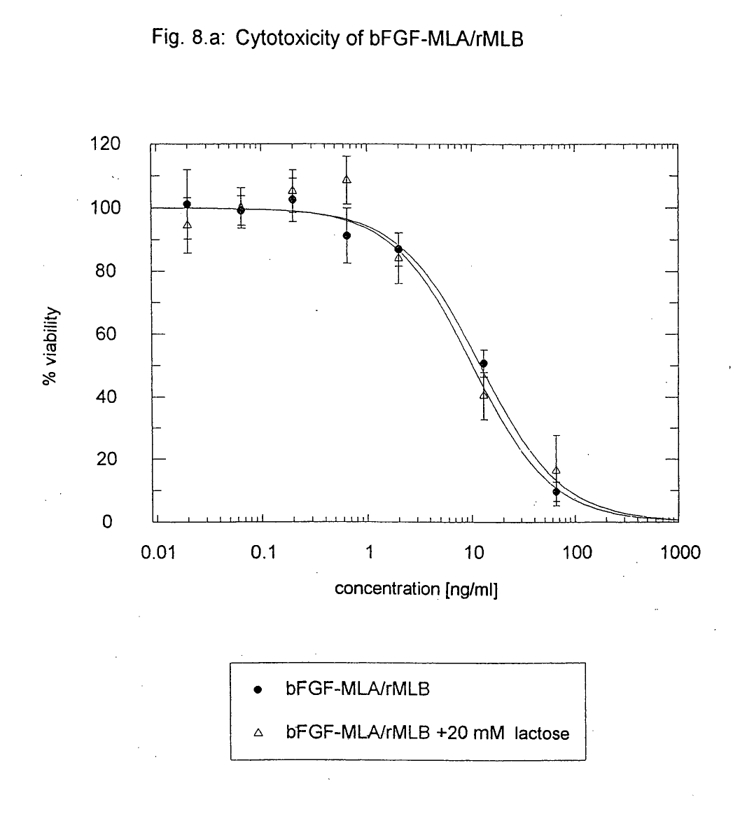

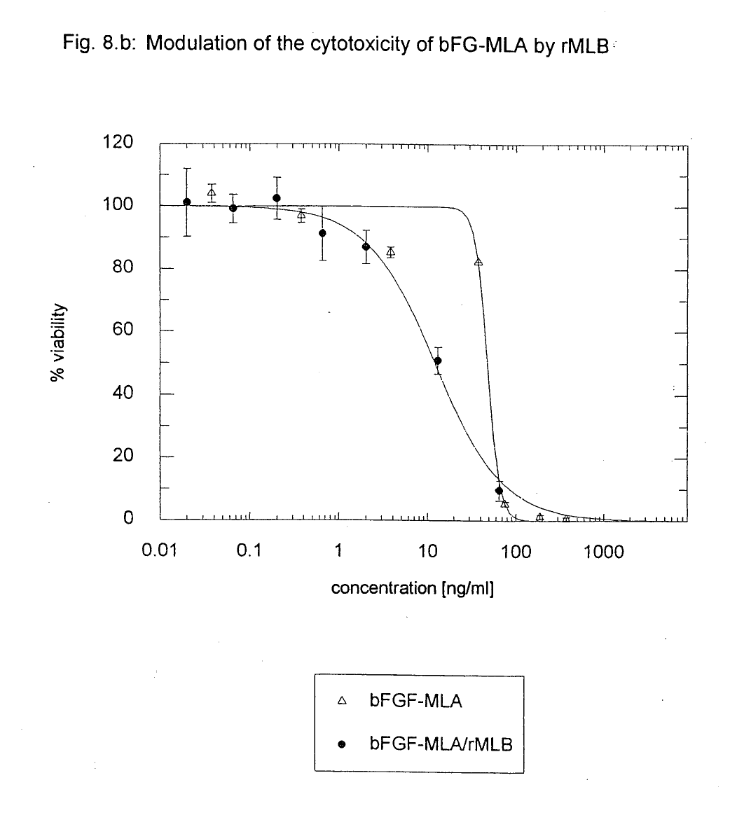

Another embodiment demonstrates the effect of the modulator module (rMLB) on an effector module (rMLA). A type TPE fusion protein, here bFGF-MLA (see above), is associated in accordance with Example 4 with rMLB via an in vitro renaturing process carried out together with rMLB (FIG. 5.a-5.b). The association during the renaturing process makes use of the specific properties of rMLB for the covalent association with rMLA by forming a disulfide bond. The required starting material in form of the two polypeptide chains can be obtained by expression in E. coli in form of cytoplasmic inclusion bodies in accordance with Example 2. The toxicity-increasing effect of the modulator module (rMLB) could be detected in an in vitro model according to Example 6. A comparison of the cytotoxicity of bFGF-MLA/rMLB with the cytotoxicity of the non-modulated TPE construct (bFGF-MLA) shows an improvement of the IC₅₀ value by factor 5, from 48 ng/ml to 10 ng/ml (FIG. 8.b). This result impressively substantiates the functionality of rMLB as modulator module. The carbohydrate binding activity of the modulator module (rMLB) modulated in the rML-ITF shown here does not have any influence on the uptake into the cells, which is proven by the fact that the addition of lactose, a competitive inhibitor of the carbohydrate binding of rMLB, does not result in an inhibition of the functionality of the associated polypeptide TPE/M (FIG. 8.a).

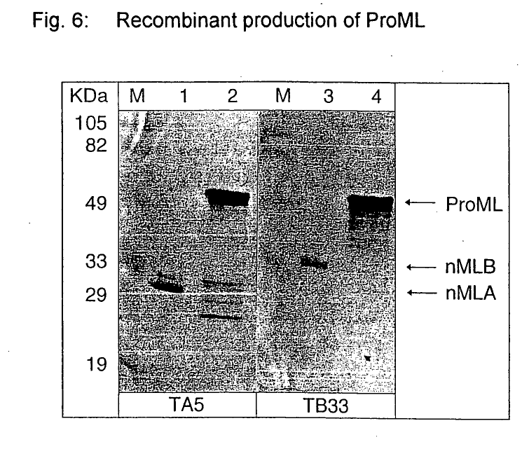

Comparative Example 1 shows the use of a polypeptide with the combination of the modules EPMT for examining the functionality of the ProML propeptide as processing module. In this specific Example a wild-type/rMLB chain is used as modulator and targeting module (MT) in whose sub-domains 1α and 2γ an intrinsic carbohydrate binding activity was left which in the present Example can be advantageously used for a poorly specific binding to glycosyl surface structures of the MOLT4 target cells and thus for targeting the construct. This targeting function is attributable on the structural level to the above-mentioned sub-domains and is thus clearly distinguishable from the modulating domains in terms of their function. This minimum model makes use of the novel properties of the recombinantly produced ProMLs, particularly starting from its propeptide. Here, the effector module (rMLA) is coupled to the modulator module (rMLB) via the propeptide of the mistletoe lectin according to Example 3. This rML-ITF, in form of ProML (FIG. 6), can be obtained via the expression in E. coli and accumulation of cytoplasmic inclusion bodies, as illustrated in Comparative Example 2.

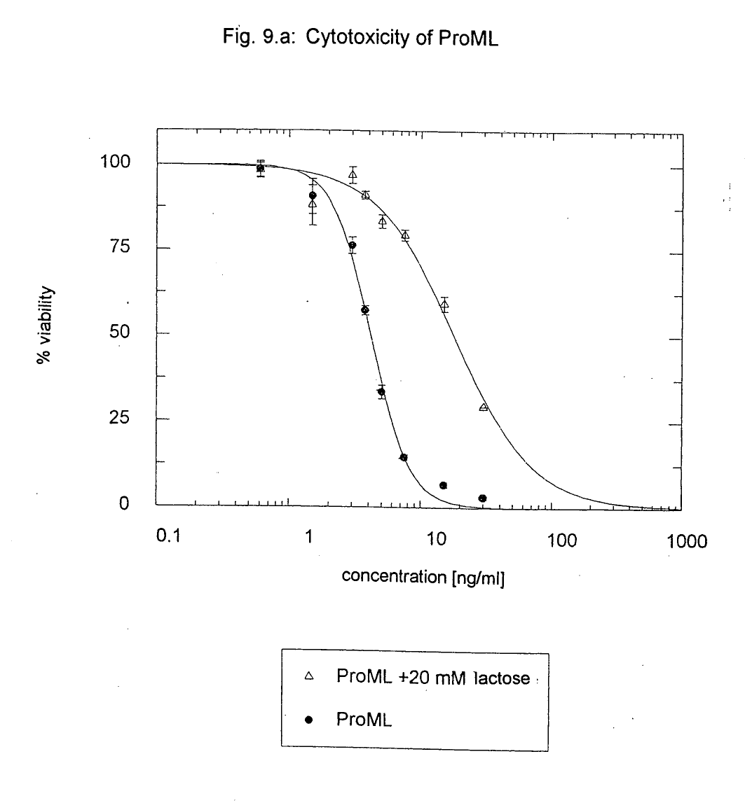

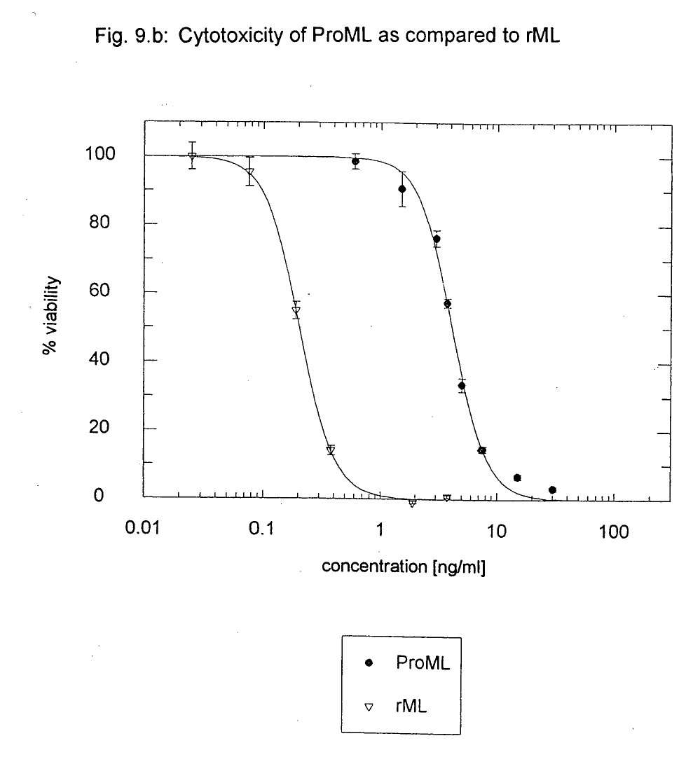

The suitability of ProML, which is depicted in comparative examples and is not part of the invention, as EPMT module is proven by the functionality test vis-à-vis immune cells of the blood such as, e.g., the human leukemia cell line MOLT-4 according to Example 9 (FIG. 9.a). The effect of ProML observed, with an IC₅₀ value of 5 ng/ml, shows the surprising property of a type II-RIP propeptide of being capable of providing a functional processing module in form of a protease-sensitive sequence which so far has not been known. Furthermore, the effector module (rMLA) is kept inactivated outside of the cell by the intact propeptide. So far it had not been possible to show this effect for other known pro-forms of type II-RIPs. In order to perform specific cell targeting it is advantageous to eliminate the unspecific binding activity of the modulator domain. For this purpose it is crucial to know the carbohydrate binding sites as well as the amino acids involved in the binding process. As described in Example 7 for the case of the B chain of the mistletoe lectin these were exchanged on nucleic acid level by mutation. Then the carbohydrate binding-inactivated rIML was produced according to the instructions in Examples 8a.-8c. by expressing the single chains and in vitro co-folding (FIG. 13). The cytotoxicity of this rML variant is, as can be seen from Example 9, drastically reduced so that in the desired low-dosage range of a potential ITF therapy a drastic reduction of the risk of side-effects as compared to immunotoxins and mitotoxins so far known can be started from (FIG. 14).

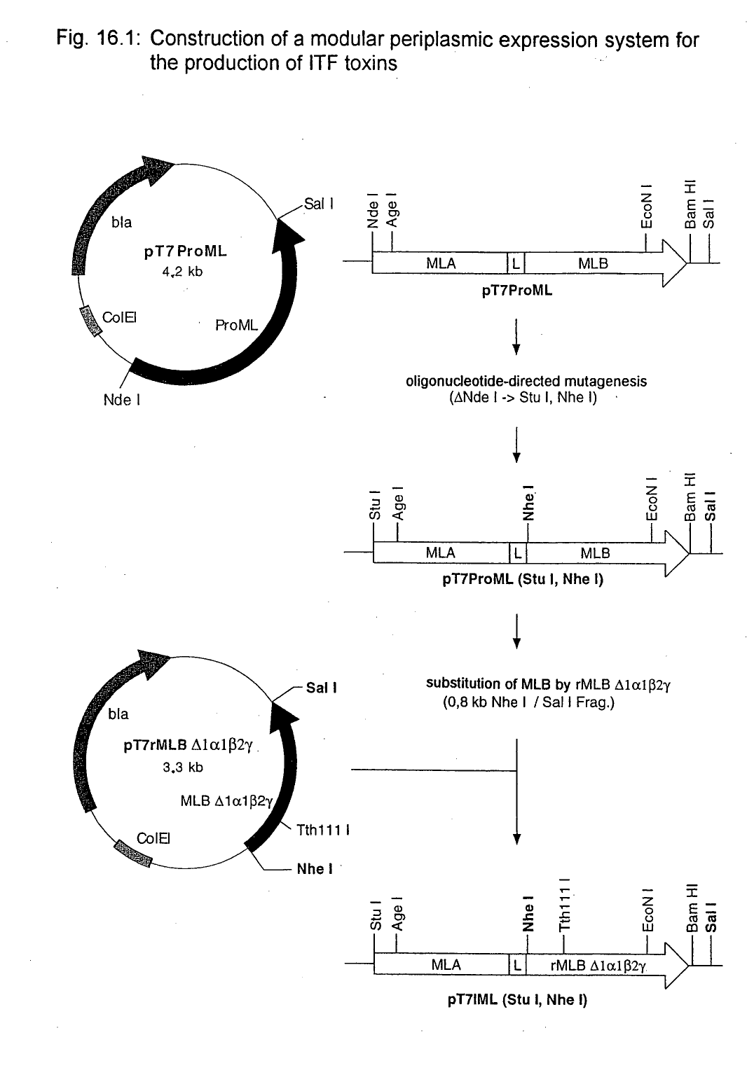

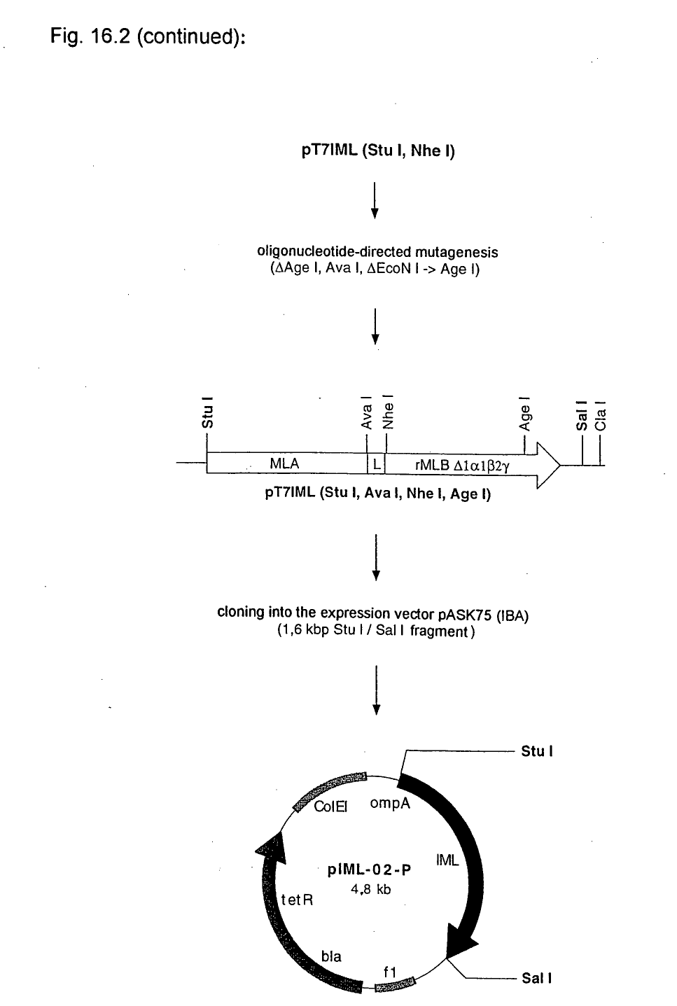

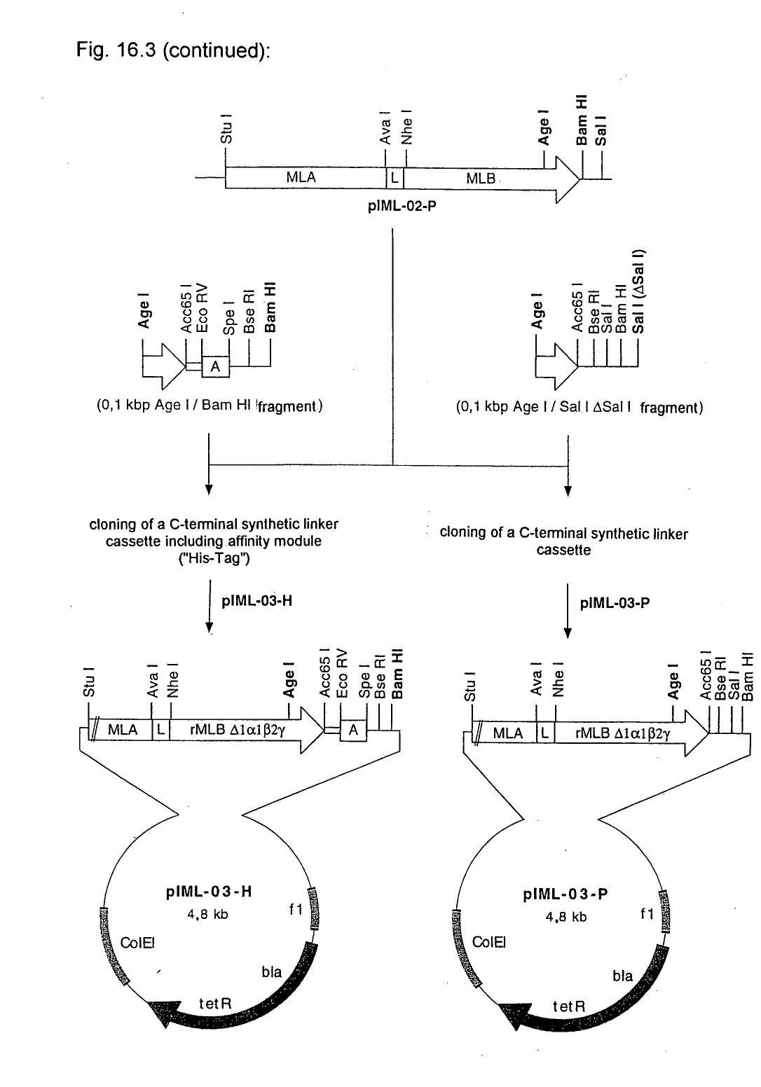

Example 10 describes how to construct vectors which serve as starting point for the construction of any ITF toxins by modular insertion of targeting modules as well as the possibility of realizing different arrangements and combinations of the individual ITF modules (FIG. 16 and FIG. 17).

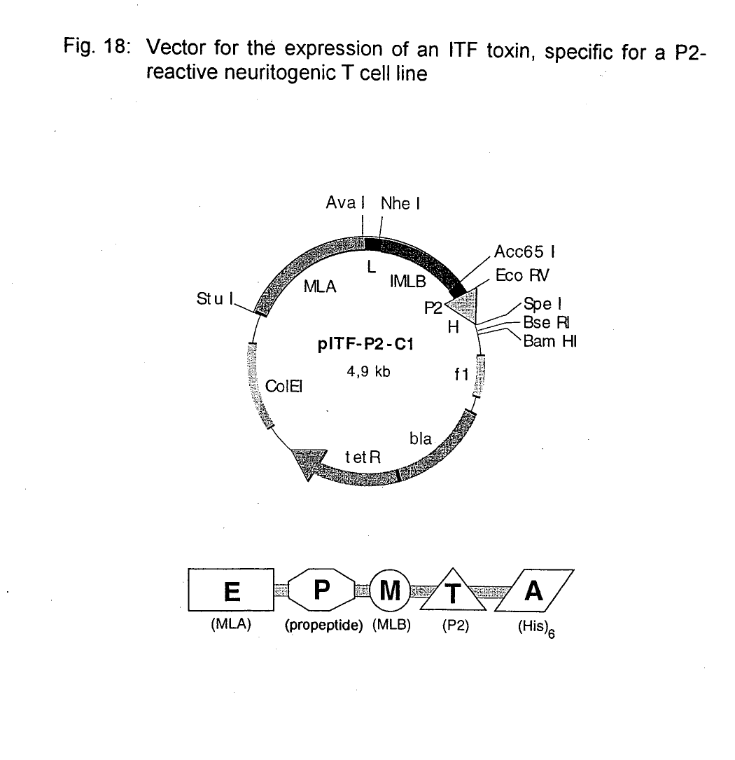

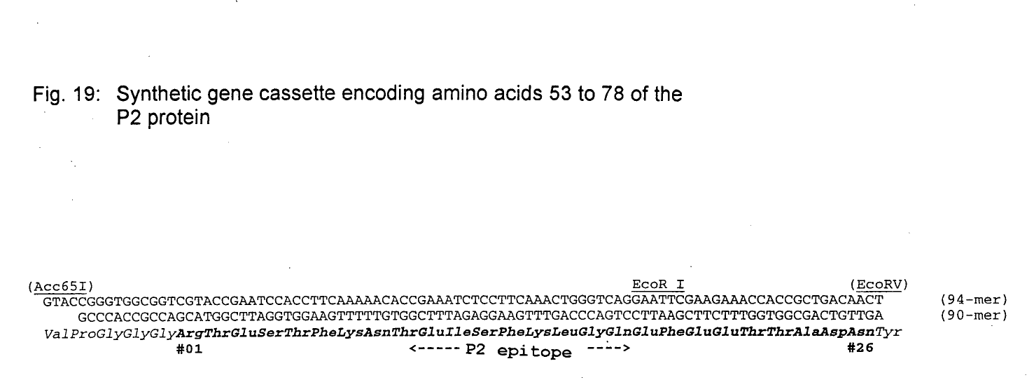

In order to demonstrate the functionality of an ITF toxin with a specific targeting module, the sequence of the neuritogenic P2 peptide (Weishaupt et al., 1995) was inserted into vector pIML-03-H (Example 11, FIGS. 17 and 18) in form of a synthetic gene fragment (FIG. 19) and expressed (Example 12.a). This ITF variant can then be purified via the affinity module, both under native (Example 12.b, FIG. 24) as well as under denaturing conditions (Example 12.b, FIG. 25) or the molecule can be renatured in vitro (Example 12.c; FIG. 27). The effectiveness of such an ITF toxin is described below in more detail.

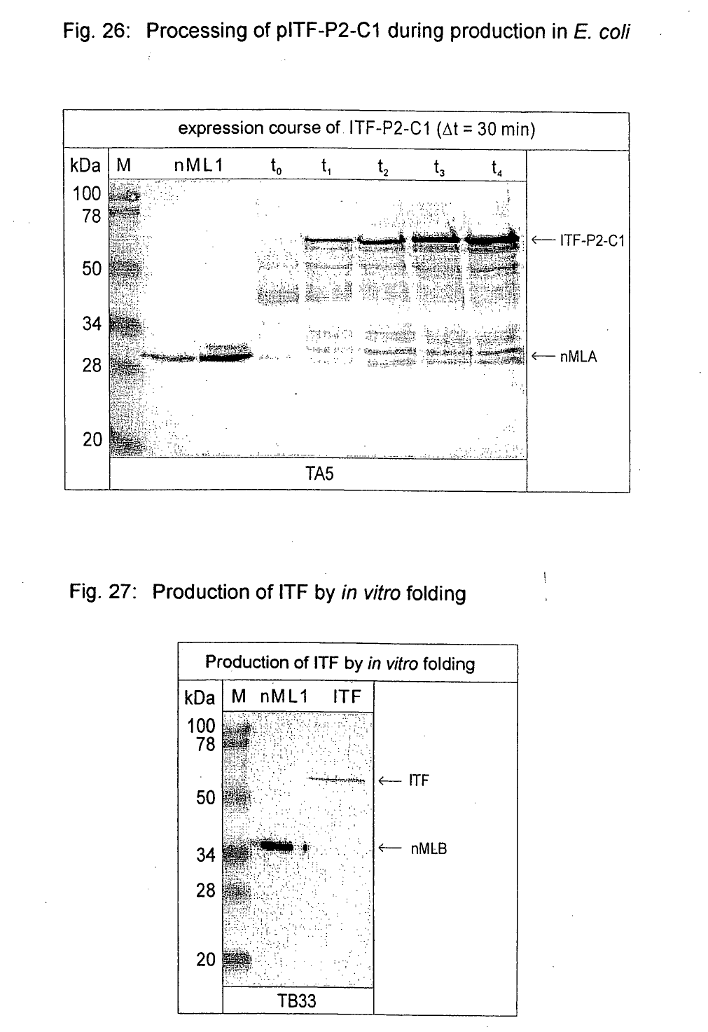

A prerequisite and at the same time one of the main problems of the development of cytotoxic substances on the basis of ribosome-inactivating proteins is the linkage of toxin, modulator and targeting modules so that they remain stably linked outside the target cells and under physiological conditions while intracellularly they are cleaved so that the toxic effect can be developed. This requirement is met by using polypeptide linkers (processing modules) which guarantee a stable linkage outside the cells while intracellularly they are hydrolytically cleaved by specific enzymes—usually proteases. In the mistletoe lectin based ITF toxins such a linker—or processing module within the meaning according to the invention—which allows for the required functionality of the toxin, could for the first time ever be successfully used. A consequence of the protease-sensitivity of the processing module used, is however, that already during the heterologous expression of the corresponding ITF genes in E. coli hydrolytically cleaved effector modules are accumulated as by-products (Example 12, FIG. 26) which have to be removed in the subsequent processing and purification of the ITFs. The ratio of degradation products can basically be reduced by using E. coli strains with a suitable protease deficiency.

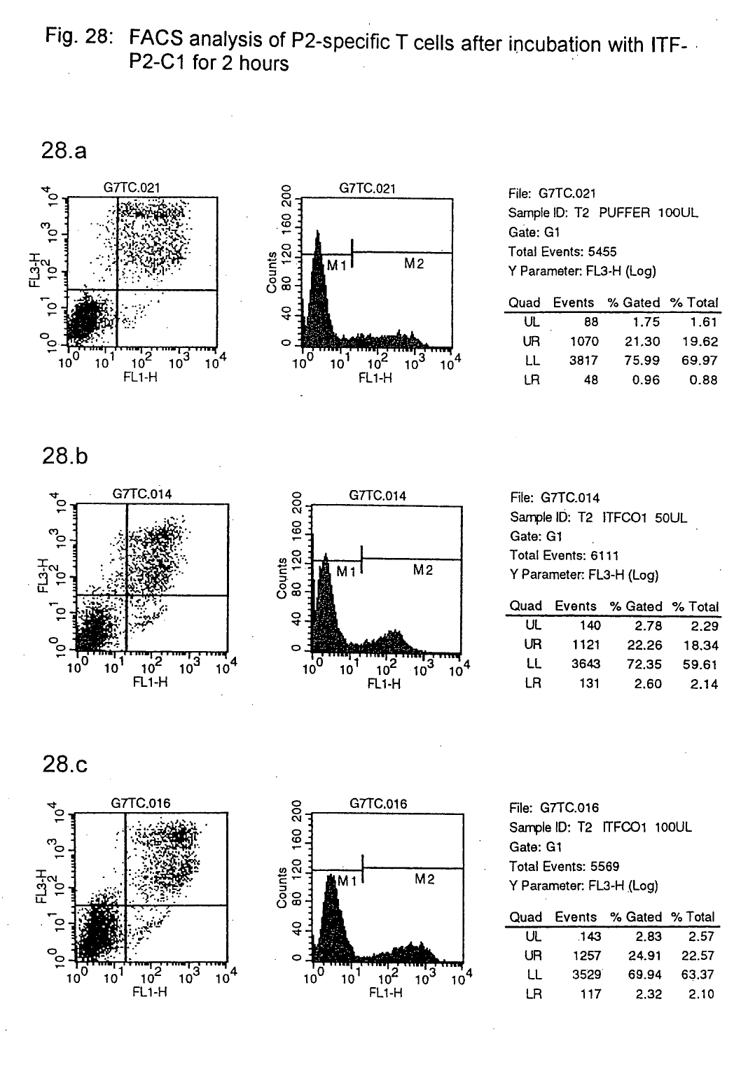

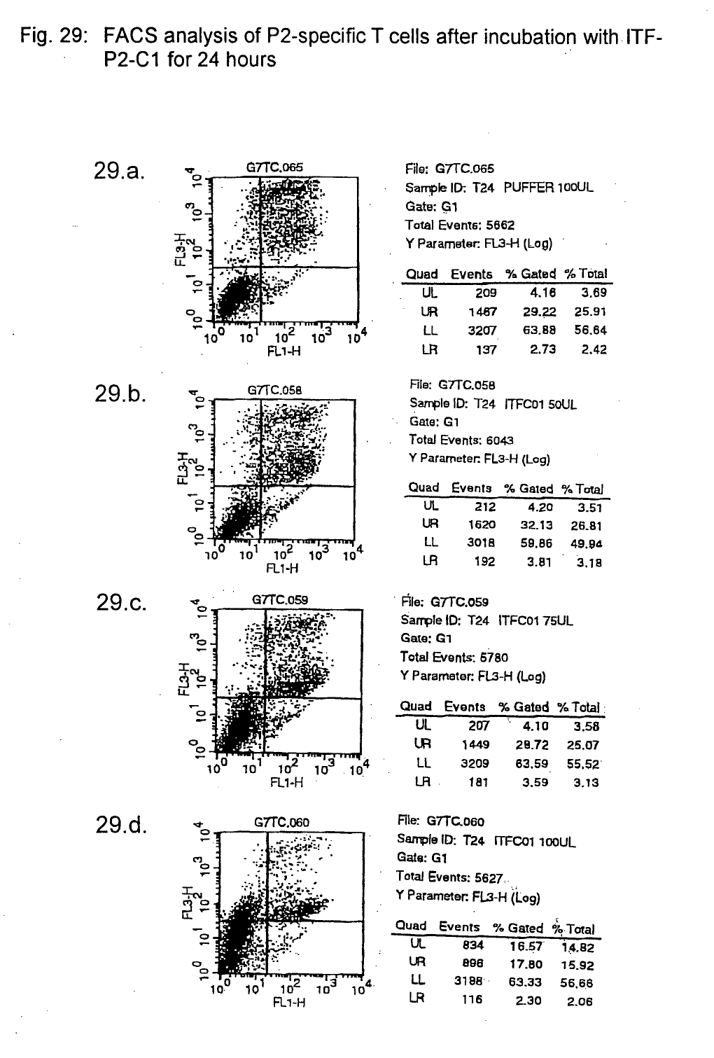

The effect of the ITF with the neuritogenic P2 peptide as targeting domain on P2-specific autoreactive T cells in vitro is for example analyzed by flow cytometry in a FACS (fluorescence activated cell sorter; Example 13). The staining method (annexin-V/propidiumiodide) allows to differentiate between apoptotic and necrotic. The measurements after 2 hrs (FIG. 28) and after 24 hrs (FIG. 29) show (detailed explanation in Example 13) that depending on the duration of treatment and concentration ITF induces both kinds of cell death.

The examples serve to illustrate the invention.

### EXAMPLE 1

### Construction of a Vector for the Heterologous Expression of a Fusion Protein of the TPE Type (bFGF-MLA) in E. Coli

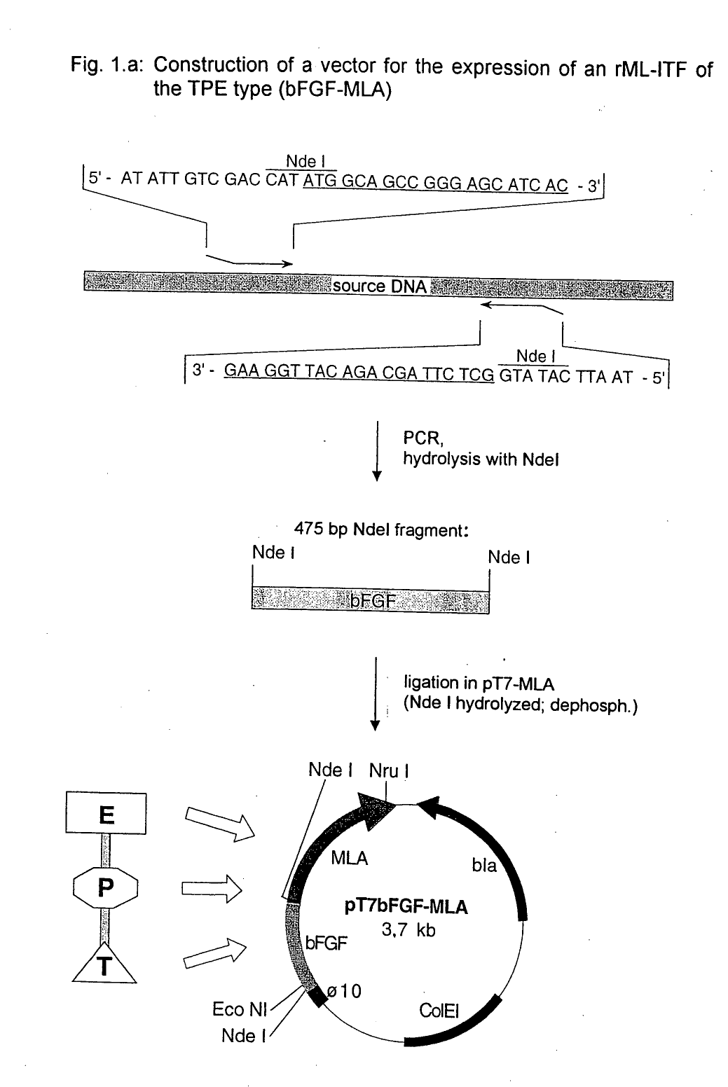

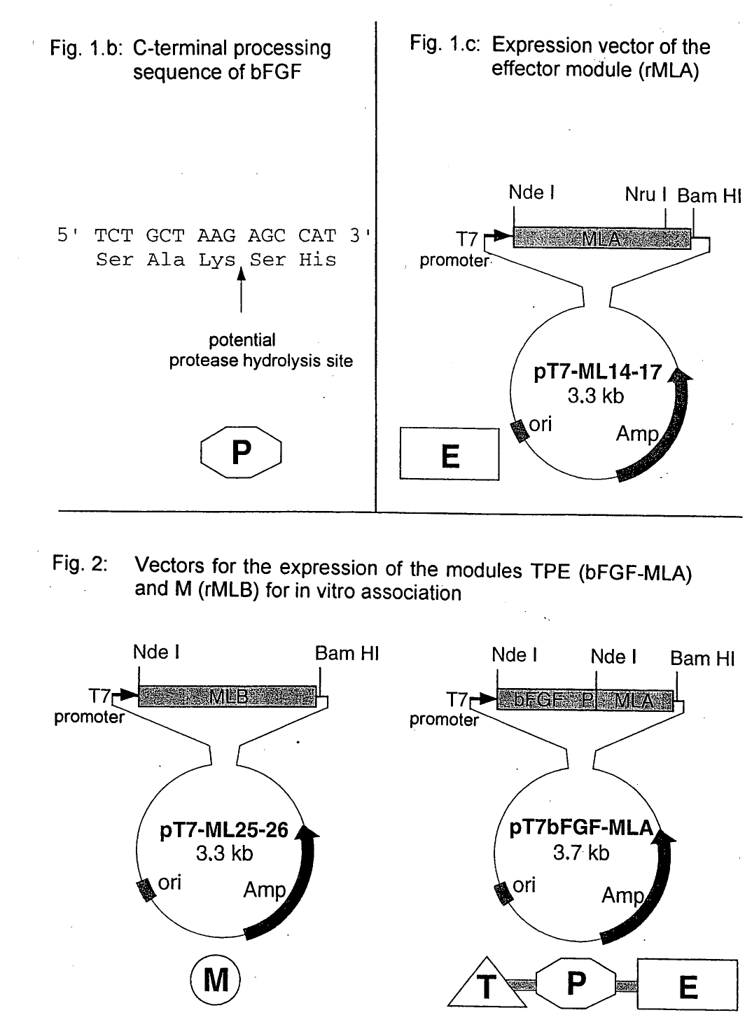

As Example of a target cell specific use of the ribosome inactivating activity of the mistletoe lectin A chain (rMLA), a fusion gene was constructed which leads to the cytoplasmic accumulation of a fusion protein, consisting of the basic fibroblast growth factor (bFGF) and rMLA in a suitable host cell (E. coli BL21). The fusion protein thus possesses the bFGF portion as targeting module and the rMLA domain as effector module. The C-terminal sequence of the bFGF contains a trypsin cleavage site (Lappi et al., 1994) and serves as processing module (FIG. 1.b).

Starting from a plasmid DNA preparation (plasmid minipreparation, Qiagen) of the plasmid pUC-bFGF (R&D Systems, Wiesbaden) which was propagated by E. coli XL1-Blue the bFGF gene (Abraham et al., 1986) was amplified by polymerase chain reaction (PCR) using bFGF-specific primers (FIG. 1.a). After hydrolysis of the amplification product with the restriction endonuclease NdeI and subsequent purification (PCR Purification Kit, Qiagen) the DNA fragment was covalently linked in a T4-ligase reaction to the likewise NdeI-hydrolyzed and dephosphorylated vector pT7-ML14-17 (FIG. 1.c), whose construction is described in detail in EP application no. 95109949.8. After transformation of the ligation mixture in E. coli XL1-Blue clones in which the desired plasmid pT7bFGF-MLA had been intracellularly established were selected by plating on ampicillin-agar. The plasmid DNA of selected clones was tested by hydrolysis with suitable restriction endonucleases for the presence in electrophoresis of predicted characteristic fragment sizes. The correct sequence of the bFGF gene from a selected positive clone was verified by nucleotide sequence analysis.

The expression vector pT7bFGF-MLA (FIG. 1.a) obtained contains the bFGF-MLA encoding fusion gene under the control of the phi 10 promoter. After induction with IPTG T7-polymerase is produced in E. coli BL21 resulting in a high transcription rate of the bFGF-MLA gene. The gene product produced can then be isolated from the soluble or the inclusion body fraction of the cells.

### EXAMPLE 2

### Construction of the Vectors for the Heterologous Production of an Associated Fusion Protein of the TPE/M Type (bFGF-MLA/rMLB)

For the production of an associated fusion protein: type TPE/M consisting of in vitro-coupled bFGF-MLA and rMLB a vector for the expression of bFGF-MLA (pT7bFGF-MLA) and a vector for the expression of rMLB (pT7-ML25-26) is required (FIG. 2). The construction of the vector pT7bFGF-MLA is described in Example 1. For the construction of the vector pT7-ML25-26 the complete, rMLB-coding sequence was amplified by specific PCR from complex genomic Viscum album DNA. Translational control elements and recognition sequences, which were used to clone the gene for rMLB into the expression vector, were introduced via non-complementary regions of the primer-oligonucleotides used (detailed description in: EP application no. 95109949.8).

### COMPARATIVE EXAMPLE 1

### Construction of a Vector for the Heterologous Expression of a Polypeptide of the EPM^{T} Type (ProML) in E. coli

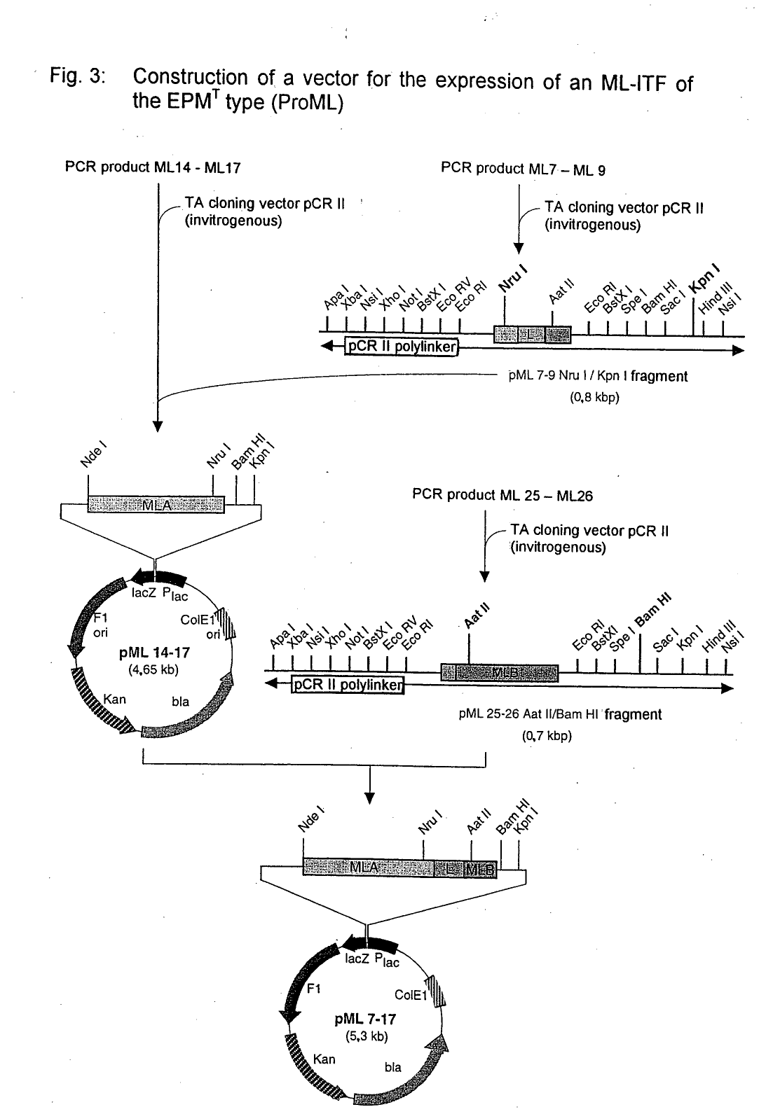

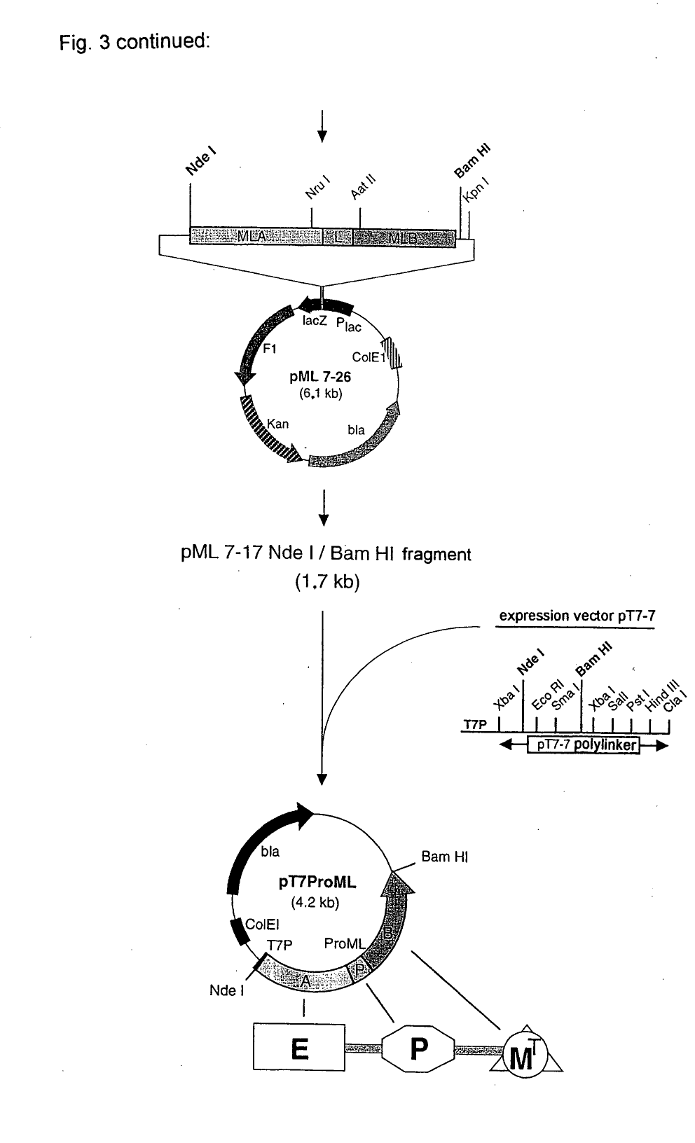

For the recombinant production of ProML—the RIP-inactive ML precursor protein synthesized in the mistletoe—the gene fragments for the rMLA (pML14-17), the propeptide (pML7-9) and the rMLB (pML25-26; detailed description in: EP application no. 95109949.8), which were isolated from the mistletoe by PCR and then cloned, were combined in two sequential ligase reactions and then cloned into expression vector pT7-7 (FIG. 3).

For this purpose, the pro-sequence was prepared on agarose gel electrophoresis after NruI/KpnI hydrolysis of the vector pML7-9 and cloned into vector pML14-17 which had been hydrolyzed with NruI/KpnI and dephosphorylated (FIG. 3). After transformation of E. coli XL1 Blue the plasmid DNA of ampicillin-resistant clones was validated for insertion of the pro-sequence by hydrolysis with NruI/KpnI. To the vector pML7-17 obtained in this manner the sequence of the rMLB chain with the pro-sequence was fused following the same strategy, however, using the restriction endonucleases AatII and BamHI, which resulted in vector pML7-26. Expression vector pT7proML was obtained according to the same steps by recloning the ProML sequence into vector pT7-7 via the restriction sites NdeI and BamHI. FIG. 11.d shows the location of the recognition sequences of the restriction endonucleases which facilitates an insertion of the modular gene cassette into a corresponding vector. In FIG. 11.d. also the arrangement of translation control elements, here of the start codons ATG as well as the stop codons TGA and TAA, as an example of cytoplasmic expression of a polypeptide of the EPM^{T} type (ProML) in E. coli is shown. The ProML gene is under the control of the phi10-T7 promoter. Upon transformation of the plasmid in E. coli BL21, which provides for the T7 polymerase gene in trans position, after induction with IPTG T7-RNA polymerase is produced the gene which is under the control of the T7 promoter is transcribed in the sense of a synergic sequence. The massive onset of the production of specific mRNA results—depending on how efficient translation is and on the protein properties—in the accumulation of the gene product in the soluble phase or in cytoplasmic inclusion bodies.

### EXAMPLE 3

### Process for the Production of a Fusion Protein (bFGF-MLA) by Soluble Expression in E. coli

The heterologous expression of the respective rML-ITF genes described in this example and in Example 6 is carried out in E. coli BL21 which possesses a chromosomally integrated T7 gene under the control of the Lac promoter. After addition of IPTG, T7-RNA polymerase mediated expression of the nucleic acid encoding the fusion protein takes place. The gene product can be obtained from the soluble (this Example) or the insoluble fraction (Example 6) of the cell disruption. The enrichment (increase/decrease) of the fusion proteins in the desired fraction can be controlled by the amount of IPTG used for induction.

For the production of recombinant bFGF-MLA fusion protein 10 ml of an E. coli BL21-(pT7bFGF-MLA; FIG. 1.a) pre-culture stationary grown in LB-Amp medium in 1000 ml LB-Amp medium were transferred to 2000 ml flasks and incubated at 37° C. and 190 rpm. When a cell density corresponding to an OD₅₇₈ of 0.9 was reached, expression of the fusion gene was induced by addition of 500 μM IPTG. Three hours after induction the cells were harvested by centrifugation (10 min, 6000 rpm, 4° C., Sorvall GS3 Rotor). The cell sediment was resuspended in buffer A (600 mM NaCl; 10 mM Tris-HCl, pH 7.4; 4° C.) and broken up by passing it twice through a “French-Press” pressure chamber (SLM Instruments) at 1500 psi. The insoluble cell components were removed by centrifugation (17000 rpm, 30 min, 4° C., Sorvall SS34 Rotor).

Soluble bFGF-MLA fusion protein with a functional bFGF portion was enriched by binding to an immobilized heparin affinity matrix (1 ml HiTrap heparin sepharose; Pharmacia) at a constant flow of 1 ml per min (Äkta chromatography device; Pharmacia). Protein that bound to the affinity matrix was eluted with buffer B (2M NaCl; 10 mM Tris-HCl; pH 7.4) and dialyzed against buffer C (50 mM NaH₂PO₄, 300 mM NaCl, 1 mM EDTA, 10% (v/v) glycerol, 0.05% (v/v) Tween-20) to prepare it for further purification. bFGF-containing degradation products as well as co-purified E. coli proteins were removed by binding the bFGF-MLA fusion protein to an anti-rMLA immunoaffinity matrix (260 μg anti-nMLA-IgG (TA5), immobilized to protein A-sepharose CL4B (Sigma, Deisenhofen) according to the method described by Harlow & Spur, 1988). The monoclonal antibody anti-nMLA-IgG TA5 (Tonevitsky et al., 1995) was provided for by the author. Like the other antibodies used herein they are producible by standard methods using the corresponding immunogen (for TA5 it is ML-1 or MLA). After two hours of incubation of the affinity matrix in the protein solution while agitating the solution at 4° C. the proteins not bound were removed by washing with buffer D (1 M NaCl; 50 mM NaH₂PO₄; pH 7.4). Bound protein was eluted with buffer E (0.1 M glycine; 300 mM NaCl; 1 mM EDTA; 10% (v/v); glycerol 0.05% (v/v) Tween-20; pH 3.0) directly in calibration buffer (1M NaH₂PO₄; pH 8.0). The identity of the protein was confirmed by Western blot analysis using the monoclonal antibodies anti-nMLA (TA5) (Tonevitsky et al., 1995) and anti-bFGF (F-6162, Sigma, Deisenhofen) and a second, alkaline phosphatase conjugated detection antibody anti-mouse IgG-IgG (Sigma, Deisenhofen; FIG. 4.a).

### EXAMPLE 4

### Production of an Associated Fusion Protein: Type TEP/M (bFGF-MLA/rMLB)

bFGF-MLA and rMLB can be provided by using the expression vectors pT7bFGF-MLA and pT7-ML25-26 (FIG. 2). For this purpose 10 ml each of an E. coli-BL21/pT7bFGF-MLA or E. coli-BL21/pT7-ML25-26 pre-culture grown stationary in LB-Amp medium in 1000 ml LB-Amp medium each were transferred to 2000 ml flask and shaken at 37° C. and 190 rpm. When a cell density corresponding to an OD₅₇₈ of 0.9 was reached, expressions were induced by addition of 500 μM IPTG. Three hours after induction the cells were harvested by centrifugation (10 min, 6000 rpm, 4° C., Sorvall GS3 Rotor).

bFGF-MLA-containing cell sediment A and the rMLB-containing cell sediment B were resuspended in 20 ml disruption buffer (20 mM NaH₂PO₄; 50 mM NaCl; 1 mM EDTA; pH 7.4; 4° C.) and broken up by passing the solution twice through a “French-Press” pressure chamber (SLM Instruments) at 1500 psi. The insoluble cell components were sedimented by centrifugation (30 min, 10000 rpm, 4° C., SS34-Rotor). Sediments A and B which contained inclusion bodies were each washed with STET buffer (8% (w/v) sucrose; 50 mM EDTA; 0.05% (v/v) Tween-20; 50 mM Tris-HCl; pH 7.4) and then dissolved under stirring for 4 hrs in 15 ml denaturing buffer (6 M guanidinium chloride; 20 mM DTT; 50 mM Tris-HCl; pH 8.0; room temperature). The insoluble cell components were sedimented by centrifugation (17000 rpm, 30 min, 4° C., Sorvall SS34 Rotor). The bFGF-MLA content of solution A was detected by Western blot analysis using immunochemical detection with monoclonal anti-bFGF antibody (F-6162, Sigma), using a bFGF standard (F-0291, Sigma, Deisenhofen). The rMLB content of solution B was detected by Western blot analysis using immunochemical detection with monoclonal anti-rMLB antibody (TB33, Tonevitsky et al., 1995) and an alkaline phosphatase conjugated anti-mouse IgG-IgG detection antibody (Sigma, Deisenhofen), using an ML1 quantitative standard (MADAUS AG, Cologne; batch no. 220793). The monoclonal antibody anti-nMLB-IgG TB33 used was provided for by the author. Like the other antibodies used herein they are producible by standard methods using the corresponding immunogen (for TB33 it is ML-1 or MLB).

For in vitro association of bFGF-MLA with rMLB a protein solution (6 M guanidinium chloride; 2 mM DTT; 50 mM Tris-HCl; pH 6.0) with a coupling agent content of 0.5 mg each was added dropwise and under stirring at a rate of about 1 ml/hr at 4° C. to folding or coupling buffer (50 mM NaH₂PO₄; 50 mM KCl; 1 mM EDTA; 10% (v/v) glycerol; 100 mM glucose; 20 mM lactose; 1 mM reduced glutathion; 1 mM oxidized glutathion; pH 8.0) of the 28-fold volume of the protein solution to a theoretical end concentration of bFGF-MLA/rMLB of 7.5 μg/ml. After stirring the solution for 24 hrs at 4° C. the insoluble components were sedimented (17000 rpm, 30 min, 4° C., Sorvall SS34 Rotor). The soluble proteins were concentrated by factor five (N₂ overpressure stirred cell with Diaflo ultrafiltration membrane YM30, Amicon) and dialyzed against chromatography buffer (20 mM NaH₂PO₄; 300 mM NaCl; 1 mM EDTA; 0.1 g/l PVP K17; pH 8.0).

The soluble and lactose-binding bFGF-MLA/rMLB was enriched by affinity chromatography on a β-lactosyl-agarose affinity matrix (No. 20364; Pierce) with a constant flow rate of 0.3 ml/min. Bound protein was eluted with 400 mM lactose-containing chromatography buffer. The eluted fraction obtained was dialyzed against storage buffer (20 mM NaH₂PO₄; 300 mM NaCl; 1 mM EDTA; 0.1 g/l PVP K17; pH 7.0). The purity of the bFGF-MLA/rMLB sample used was documented by PAGE and subsequent silver staining (FIG. 5.a). The identity of the sample's band was confirmed by Western blot analysis with the monoclonal antibodies anti-bFGF (F-6162, Sigma) and anti-rMLB (TB33, Tonevitsky et al., 1995) as well as an alkaline phosphatase conjugated anti-mouse IgG-IgG detection antibody (Sigma, Deisenhofen; FIG. 5.b).

### COMPARATIVE EXAMPLE 2

### Provision of an rML-ITF of the EPMT Type (ProML) by Expression in E. coli in Form of Cytoplasmic Inclusion Bodies

For the production of recombinant ProML 10 ml of an E. coli-BL21/pT7proML pre-culture grown stationary in LB-Amp medium in 1000 ml LB-Amp medium were transferred to 2000 ml flasks and shaken at 37° C. and 190 rpm. When a cell density corresponding to an OD₅₇₈ of 0.9 was reached, the expression was induced by addition of 500 μM IPTG. Three hours after induction the cells were harvested by centrifugation (10 min, 6000 rpm, 4° C., Sorvall GS3 Rotor).

The cell sediment was resuspended in 20 ml disruption buffer (20 mM NaH₂PO₄; 50 mM NaCl; 1 mM EDTA; pH 7.4; 4° C.) and broken up by passing it twice through a “French-Press” pressure chamber (SLM Instruments) at 1500 psi. The insoluble cell components were sedimented by centrifugation (30 min, 10000 rpm, 4° C., SS34-Rotor). The sediment which contained inclusion bodies was five times washed with STET buffer (8% (w/v) sucrose; 50 mM EDTA; 0.05% (v/v) Tween-20; 50 mM Tris-HCl; pH 7.4) and then dissolved under stirring for 4 hours in 15 ml denaturing buffer (6 M guanidinium chloride; 20 mM DTT; 50 mM Tris-HCl; pH 8.0; room temperature). The insoluble cell components were removed by centrifugation (17000 rpm, 30 min, 4° C., Sorvall SS34 Rotor).

The ProML content of this solution was detected by Western blot analysis using immunochemical detection with monoclonal anti-rMLA antibody (TA5, Tonevitsky et al., 1995) using an ML1 quantitative standard (MADAUS AG, Cologne; batch no. 220793). The protein solution was rebuffered by gel filtration (PD10, Pharmacia) to renaturing conditions (6 M guanidinium chloride; 10 mM NaH₂PO₄; pH 4.5) and adjusted to a ProML concentration of 400 μg/ml. Renatured ProML was obtained by adding the protein solution dropwise (about 1 ml/hr) under stirring to the 20-fold volume folding buffer (50 mM KCl; 1 mM EDTA; 100 mM glucose; 10 mM lactose; 10% (v/v) glycerol; 3 mM oxidized glutathion; 0.6 mM red. glutathion; 50 mM Tris-HCl; pH 8.5; 4° C.). The supernatant obtained after centrifugation (17000 rpm, 30 min, 4° C.) was concentrated at 4° C. by factor 4 (N₂ overpressure stirred cell with Diaflo ultrafiltration membrane YM30, Amicon) and again subjected to centrifugation (17000 rpm, 30 min, 4° C.). Then the sample was dialyzed against the storage buffer (300 mM NaCl; 1 mM EDTA; 100 mg/l PVP-K17; 20 mM NaH₂PO₄; pH 8.0; 4° C.). Yield and identity of the renatured ProMLs was confirmed by Western blot analysis, a PAGE carried out under reducing conditions using the MLA and MLB specific monoclonal antibodies TA5 and TB33 (Tonevitsky et al., 1995) as well as an alkaline phosphatase conjugated anti-mouse IgG-IgG detection antibody (Sigma, Deisenhofen; FIG. 6).

For selectively enriching ProML with a functionally renatured B chain portion the protein solution was diluted 1/10 in chromatography buffer (100 mM NaCl; 1 mM EDTA; 100 mg/l PVP-K17; 0.05% (w/v) BSA; 50 mM Na acetate/glacial acetic acid; pH 5.6; 4° C.), bound to a β-lactosyl-agarose affinity matrix (No. 20364, Pierce) with a constant flow rate of 0.3 ml/min and eluted with chromatography buffer-containing 400 mM lactose. The eluted fraction obtained was dialyzed against storage buffer (20 mM NaH₂PO₄; 300 mM NaCl; 1 mM EDTA; 0.1 g/l PVP-K17; pH 7.0).

### EXAMPLE 5

### Functionality of a Fusion Protein of the TPE Type (bFGF-MLA) Vis-à-Vis Target Cells

The cytotoxicity of the fusion protein bFGF-MLA was determined vis-à-vis a mouse cell line B16 (DKFZ Heidelberg, TZB-No.: 630083) in a range of concentration of 375 ng/ml to 37.5 pg/ml. For this purpose a 96-well microtiter plate (Nunc, Wiesbaden) was inoculated with 1500 B16 cells each in 100 μl culture medium each (RPMI-1640 (R-7880, Sigma) plus 5% FKS). The concentration of the bFGF-MLA solution used for this purpose was determined by Western blot analysis using immunochemical detection with monoclonal anti-bFGF antibody (F-6162, Sigma) using a bFGF-containing solution of known bFGF content (F-0291, Sigma).

After 24 hours of incubation in an incubator (37° C.; 5% CO₂) it was verified under the microscope whether the cells adhered to the culture plate. 10 μl of the supernatant were replaced by culture medium which contained bFGF-MLA fusion protein in serial dilutions and six replicas were made per bFGF-MLA dilution factor. After further 72 hours of incubation the cytotoxic effect was quantitated by determining the viability of the cells according to the WST-1 method (Scudiero et al., 1988). The color reaction was evaluated by determining the optical density at a wave length of 490 nm (reference wave length: 690 nm) with a microtiter plate photometer (MWG-Biotech, Ebersberg). The IC₅₀ value (the bFGF-MLA concentration that results in a reduction of the viability vis-à-vis a positive control by 50%) was obtained by a 4 parameter curve fitting to the measured values. The bFGF-MLA fusion protein showed a cytotoxic activity with an IC₅₀ value of 48 ng/ml (FIG. 7).

For a verification of the cytotoxic effect of the bFGF-MLA fusion protein by bFGF-mediated internalization via a specific binding to bFGF receptor molecules present on the surface of the B16 cells the cytotoxic effect of rMLA on B16 cells in a concentration range of 4 μg/ml to 200 pg/ml was determined using the above-described method (FIG. 7). In the concentration range of the IC₅₀ value of the bFGF-MLA fusion protein of 48 ng/ml no cytotoxic effect of rMLA could be observed. In the highest concentration of 4 μg/ml used a viability of the B16 cells of more than 60% could be observed, which can be interpreted as a commencing cytotoxicity of rMLA via unspecific uptake.

A substance (bFGF-MLA) could be obtained by fusion of the effector module to the processing module and the targeting module which substance is capable of killing target cells with an IC₅₀ value of 48 ng/ml. In contrast thereto, the effector module (rMLA) does not exhibit an unspecific toxicity up to an examined concentration of 4 μg/ml. The toxicity of the effector module could be increased at least by factor 100 by way of the fusion to the processing and the targeting module.

### EXAMPLE 6

### Functionality of an Associated Fusion Protein of the TPE/M Type (bFGF-MLA/rMLB) Vis-à-Vis Target Cells

The cytotoxicity of the in vitro associated fusion protein (bFGF-MLA coupled to rMLB under co-folding) determined vis-à-vis the mouse cell line B16 (DKFZ Heidelberg, TZB-No.: 630083) in a concentration range of 65 ng/ml to 1 pg/ml, the concentrations having been determined by an “Enzyme Linked Lectin Assay” (ELLA; Vang et al., 1986).

For this purpose, a 96-well microtiter plate (Nunc, Wiesbaden) was inoculated with 1500 B16 cells each in 100 μl culture medium (RPMI-1640 (R-7880, Sigma) each plus 5% FKS). After 24 hours of incubation in an incubator (37° C., 5% CO₂) it was verified under the microscope whether cells adhered. 10 μl of the supernatant were replaced by a culture medium which contained bFGF-MLA/rMLB fusion protein in serial dilutions and six replicas were made per bFGF-MLA dilution factor. After further 72 hours incubation the cytotoxic effect was quantitated by determining the viability of the cells according to the MTT method (M-5655, Boehringer; Mossmann, 1983).

The color reaction was evaluated by determining the optical density at a wave length of 562 nm (reference wave length: 690 nm) with a microtiter plate photometer (MWG-Biotech, Ebersberg). The IC₅₀ value (the bFGF-MLA/rMLB concentration that results in a reduction of the viability vis-à-vis a positive control by 50%) was obtained by a 4 parameter curve fitting to the measured values.

The rMLB associated fusion protein bFGF-MLA shows a cytotoxic effect with an IC₅₀ value of 10 ng/ml (FIG. 8.a). The cell-specific uptake via binding to bFGF-specific cell surface receptor was verified by a parallel test which was identical except for the presence of 20 mM lactose in the medium. The cytotoxic effect is not attenuated for bFGF-MLA/rMLB (FIG. 8.a).

The IC₅₀ value as standard for the specific toxicity of the TPE fusion protein (bFGF-MLA) could be increased for bFGF-MLA/rMLB from 48 ng/ml to 10 ng/ml by adding the modulator (FIG. 8.b). It could be shown that the toxicity of the effector module (rMLA) specified via a targeting module (bFGF) can be increased by several times using a modulator module (rMLB).

### COMPARATIVE EXAMPLE 3

### Cytotoxicity of a Polypeptide of the EPMT Type (ProML) Vis-à-Vis Human Lymphatic Leukemia Cells

The development of the cytotoxic activity of ProML was measured using the human mononuclear lymphatic leukemia cell line MOLT-4 (European Collection of Animal Cell Cultures No. 85011413) in a concentration range of 0.6 ng/ml-30 ng/ml.

MOLT-4 cells were cultivated in serum-free MDC-1 medium (PAN SYSTEMS, Aidenbach) and adjusted for the test to a cell density of 1.6×10⁵ cells/ml at a viability of >98%. 90 μl (corresponding to 18000 MOLT-4 cells) were seeded per well of a 96-well microtiter plate (Nunc, Wiesbaden) and mixed with 10 μl each of ProML-containing MDC-1 medium, in increasing dilution factors. The ProML content of the solution used was first quantitated by ELLA analysis (Vang et al., 1986) using an ML1 quantitative standard (MADAUS AG, Cologne, batch no. 220793). Preparations with pure medium and with ProML storage buffer added were used as controls. Six replicas were made for each ProML concentration and for each control. The cells were incubated for 72 hours at 37° C. and 5% CO₂ in an incubator.

The cytotoxic effect was quantitated by determining the viability of the cells according to the WST-1 method (Scudiero et al., 1988). The color reaction was evaluated by determining the optical density at a wave length of 490 nm (reference wave length: 690 nm) with a microtiter plate photometer (MWG-Biotech, Ebersberg). The IC₅₀ value (the ProML concentration which results in a reduction of the viability (or the optical density) vis-à-vis the positive control by 50%) was obtained by a 4 parameter curve fitting to the measured values. ProML develops cytotoxicity vis-à-vis MOLT-4 cells with an IC₅₀ value of 5 ng/ml. The fact that this effect is based on a specific rMLB mediated endocytosis is confirmed by an increase of the IC₅₀ value to 26 ng/ml in the presence of 20 mM lactose (FIG. 9.a).

The result surprisingly shows the potency of the natural pro-sequence to function as effective processing module. The toxicity of ProML with an IC₅₀ value of 5 ng/ml has been attenuated vis-à-vis the RIP-active rML with an IC₅₀ value of 200 pg/ml by factor 25 (FIG. 9.b). Together with its property of keeping the effector domain inactive in the non-processed condition ProML possesses ideal properties for its use as EPM component in medicaments.

### EXAMPLE 7

### Construction of an rMLB-Derived Modulator Module with Reduced Carbohydrate Affinity

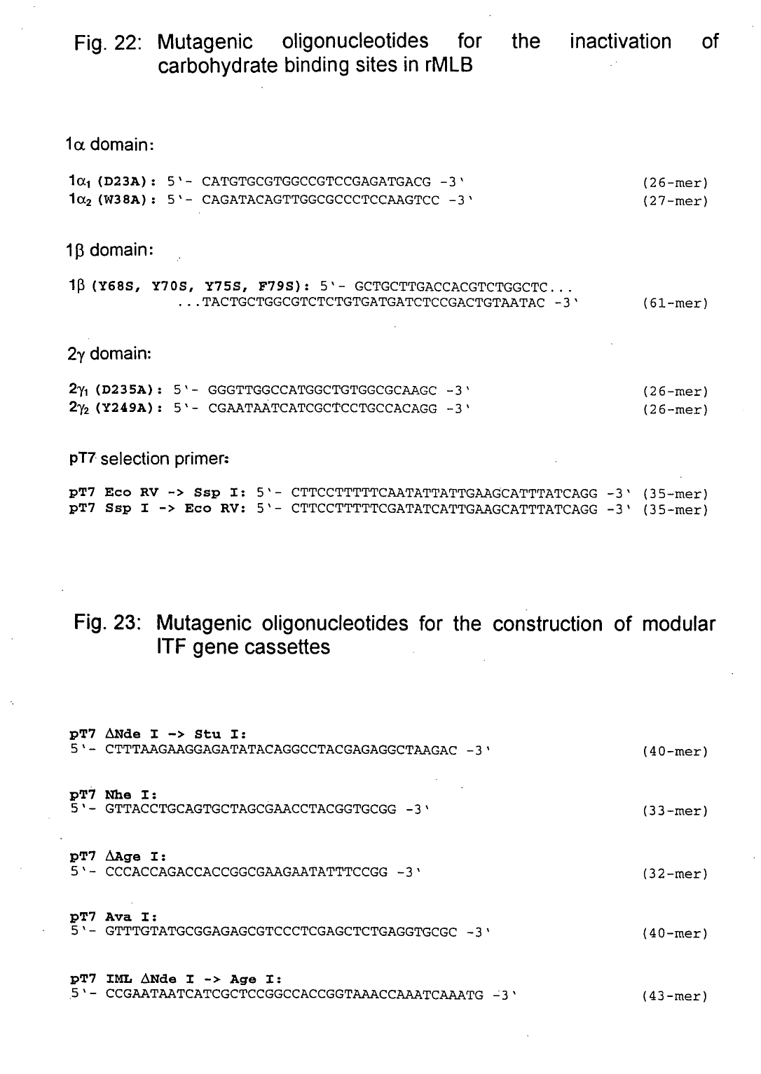

On the basis of the information regarding ricin in the literature as well as additional computer-aided calculations of the field of force a total of eight amino acids was identified in the mistletoe lectin B chain for which a functional role in carbohydrate binding could be assumed to be likely. For this reason the codons for these amino acids were exchanged by successively performed oligonucleotide-directed mutageneses according to Deng et al., 1992 (Chamäleon Mutagenesis Kit, Stratagene) for alanine (D23 for A, W38 for A, D235 for A, Y249 for A) or serine codons (Y68 for S, Y70 for S, Y75 for S, F79 for S; FIG. 15, FIG. 22.a). As selection primer the primers “pT7 Ssp I->Eco RV” and “pT7 Eco RV->Ssp I” (FIG. 22.b) were alternately used. The plasmid DNA of individually selected clones (E. coli X11 Blue) obtained by the mutageneses was examined by nucleotide sequence analysis for the presence of the expected mutated DNA sequence.

### EXAMPLE 8

### Production of Recombinant Mistletoe Lectin Variant (8.a-8.c)

(8.a) Expression of rMLA in E. Coli in Form of Insoluble Inclusion Bodies and Preparation of an rMLA-Containing Guanidinium Chloride Solution

For the expression of recombinant mistletoe lectin A chain 1000 ml LB/Amp medium (in 2 l aeration-causing flask) were inoculated with 10 ml of a stationary grown pre-culture (50 ml) and cultivated at 37° C. and 190 rpm. The growth of the culture was observed by turbidimetry at 578 nm. When an OD₅₇₈ of 1.0 was reached, the expression of the rML genes was induced by adding 0.5 mM IPTG. Two hours later, the cells were harvested (20 min, 6000 rpm, 4° C., Beckmann JA10 Rotor). The cell sediment thus obtained was resuspended in 20 ml disruption buffer (100 mM NaCl, 1 mM EDTA, 5 mM DTT, 1 mM PMSF, 50 mM Tris/HCl pH 8.0) and twice broken up in an N₂ gas pressure homogenizer at 1500 psi. The rMLA inclusion bodies were sedimented by subsequent centrifugation (30 min, 10000 rpm, 4° C., Beckmann JA20). The sediment was washed tree times with 30 ml STET buffer each (50 mM EDTA, 8% (w/v) glucose, 0.05% (v/v) Tween-20, 50 mM Tris/HCl, pH 7.4 according to Babbitt et al., 1990) to eliminate E. coli proteins. After dissolving the remaining cell sediments in guanidinium chloride (6 M GuHCl, 100 mM DTT, 50 mm Tris/HCl, pH 8.0) for 12 hours at room temperature insoluble components were sedimented by centrifugation (17000 rpm, 30 min, 4° C., JA20 Rotor) and discarded. The rMLA content of the solution obtained was determined by Western blot analysis using the nMLA- and rMLA-specific monoclonal antibody (TA5) and a standardized nML1 sample.

(8.b) Expression of rMLB Δ1α1β2γ in E. coli in Form of Inclusion Bodies and Preparation of an rMLB Δ1α1β2γ-Containing Guanidinium Chloride Solution

For the expression of recombinant mistletoe lectin B chain (rMLB) or the non-carbohydrate binding rMLB Δ1α1β2γ variant 1000 ml LB/Amp medium (in 2 l Schikanekolben) each were inoculated with 10 ml of a stationary grown pre-culture (50 ml) and cultivated at 37° C. and 190 rpm. The growth of the culture was observed by turbidimetry at 578 nm. When an OD₅₇₈ of 1.0 was reached, the expression of the rMLB or of the rMLB Δ1α1β2γ gene was induced by adding 0.5 mM IPTG. Four hours after induction the cells were harvested (20 min, 6000 rpm, 4° C., Beckmann JA10 Rotor). The cell sediment thus obtained was resuspended in 20 ml disruption buffer B (50 mM NaCl, 1 mM EDTA, 5 mM DTT, 1 mM PMSF, 20 mM NaH₂PO₄, pH 7.2) and twice broken up with an N₂ gas pressure homogenizer at 1500 psi. The rMLB-containing inclusion bodies were sedimented by subsequent centrifugation (30 min, 10000 rpm, 4° C., Beckmann JA20). The sediment was washed three times with 30 ml STET-buffer each (50 mM EDTA, 8% (w/v) glucose, 0.05% (v/v) Tween-20, 50 mM Tris/HCl, pH 7.4 according to Babbitt et al., 1990) to eliminate E. coli proteins. After dissolving the remaining cell sediment in guanidinium chloride (6 M GuHCl, 100 mM DTT, 50 mm Tris/HCl, pH 8.0) for 12 hrs at room temperature insoluble components were sedimented by centrifugation (17000 rpm, 30 min, 4° C., JA20 Rotor) and discarded. The rMLB content of the solution obtained was determined by Western blot analysis using the nMLB- and rMLB-specific monoclonal antibody (TB33) and a comparative sample with known nML1 content. The same method can be used to obtain rMLB (amino acid sequence identical to that of natural mistletoe lectin).

(8.c) Process for Producing rIML Holotoxin by In Vitro Folding

The process serves to fold and simultaneously couple the non-carbohydrate binding rMLB variant (rMLB Δ1α1β2γ) to rMLA for obtaining a recombinant (holo) mistletoe lectin with reduced carbohydrate affinity (rIML).

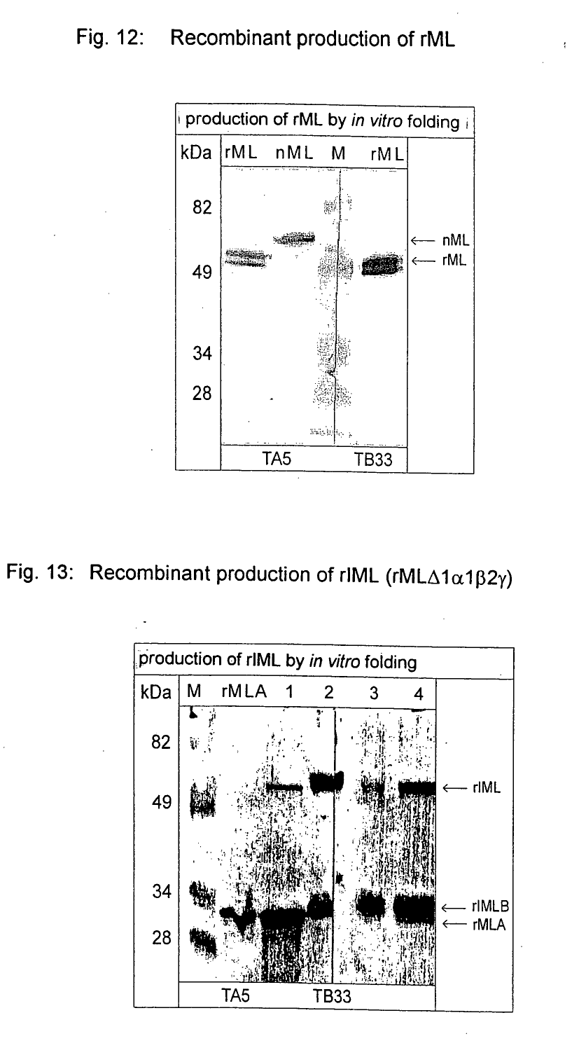

The denatured components of rIML, rMLA and rMLB Δ1α1β2γ (see Example 8.a and Example 8.b) which are dissolved in GuHCl were adjusted to a concentration of 200 μg/ml, mixed in equal portions and adjusted by gel permeation (PD10, Pharmacia) to defined buffer conditions (6 M GuHCl, 2 mM DTT, 50 mm Tris/HCl, pH 8.0). The in vitro folding and association was carried out by slowly adding this solution dropwise to a 30-fold volume of folding buffer (50 mM KCl, 1 mM EDTA, 100 mM glucose, 20 mM lactose, 10% (v/v) glycerol, 1 mM reduced glutathion, 1 mM oxidized glutathion, 50 mM NaH₂PO₄, pH 8.0) under constant stirring at 4° C. for about 12 hours. Afterwards, insoluble components were sedimented (30 min 17000 rpm, 4° C., JA20 Rotor) and the content of soluble rIML of the supernatant which was concentrated about 10-fold was quantitated by Western blot analysis (FIG. 13). For the production of soluble rML the same method was used, however, instead of rMLB, Δ1α1β2γ rMLB was used which is identical to the amino acid sequence of the natural mistletoe lectin B chain (FIG. 12).

### EXAMPLE 9

### Determination of the Cytotoxicity of rIML Vis-à-Vis Human Lymphatic Leukemia Cells

The cytotoxicity vis-à-vis MOLT-4 cells of holo-protein rIML from inactivated B chain (rMLB Δ1α1β2γ) which was produced by in vitro folding and covalently linked via a disulfide bond to the recombinant mistletoe lectin A chain (rMLA) was determined in the cytotoxicity test in a concentration range of 100 pg/ml-100 ng/ml according to the method described in Comparative Example 3. The respective IC₅₀ value of rIML of 25 ng/ml is reduced by factor 350 (FIG. 14) vis-à-vis the IC₅₀ value of rML which is used for reference and which is identical to the natural example nML except for the glycosylation and is about 40 times higher than the toxicity of the recombinant A chain alone (IC₅₀>1 μg/ml). From this behavior it can be concluded that the lectin activity of the B chain which results in an unspecific uptake of the toxin in any cell type whatsoever could at least be substantially attenuated by the amino acid exchanges performed.

### EXAMPLE 10

### Construction of Expression Vectors with Modularly Arranged Gene Cassettes for Effector, Processing and Modulator and Affinity Modules

Starting from vector pT7-ProML which contains the structural gene for pro-mistletoe lectin corresponding gene cassettes were generated by modification of the DNA sequence by oligonucleotide-directed mutagenesis (Deng et al., 1992) which can be exchanged by relatively simple methods for other gene cassettes with alternative affinity, effector, modulator and processing domains. These modifications allow to easily insert targeting modules before or after each module. The periplasmic cell compartment of E. coli fulfills to a high extent the requirements of a disulfide bond containing protein to the microenvironment necessary for the formation of functional tertiary structures. Therefore, the gene cassettes were inserted in this example also in a periplasmic expression vector.

Starting from the structural gene for ProML the Nde I recognition sequence present at the 5′ end of the structural gene of the effector module rMLA was exchanged for a Stu I recognition sequence using oligonucleotide-directed mutagenesis (Deng et al., 1992), and a Nhe I recognition sequence introduced at the 5′ end of the structural gene of the modulator (MLB; FIG. 16.1 top; FIG. 23a-b). The (carbohydrate binding) modulator module rMLB was then exchanged for a modulator module rIMLB (rMLB Δ1α1β2γ) which does not possess carbohydrate affinity and originates from vector pT7rMLB Δ1α1β2γ (see FIG. 16.1 bottom). For this purpose the vectors pT7ProML (Stu I, Nhe I) and pT7rMLB Δ1α1β2γ were each hydrolyzed with the restriction endonucleases Nhe I and Sal I. Then the 0.8 kbp structural gene for rIMLB was separated electrophoretically on an agarose gel (1% w/v) from the expression vector and extracted from the gel material (Qiagen Gel-Extraction Kit). Then the gel fragment so prepared was covalently linked in a T4 ligase reaction to the cleaved and additionally dephosphorylated vector pT7ProML (Stu I, Nhe I). After transformation of the ligation mixture in E. coli XL1 Blue and plating it on ampicillin-containing selective agar the DNA was prepared form 5 ml overnight cultures of selected cultured E. coli clones (Qia-Präp Kit, Qiagen). The DNA from those cones containing the desired vector pT7IML (Stu I, Nhe I) can be linearized by adding the restriction endonuclease Tth111 I and identified by the presence of a characteristic 3.3 kb band in agarose gel electrophoresis (FIG. 16.1 bottom). The thus obtained vector pT71mL (Stu I, Nhe I) was again modified by oligonucleotide-directed mutagenesis such that the Age I recognition sequence in the 5′ of the MLA gene was removed, an Eco NI recognition sequence near the 3′ end of the IML structural gene was converted to an Age I recognition sequence, and an Ava I recognition sequence was introduced at the 3′ end of the MLA gene (FIG. 16.2, FIG. 23.c-23.e). The thus obtained vector pT7IML (Stu I, Ava I, Nhe I, Age I) was mixed in a molar ratio of 3:1 with the periplasmic expression vector pASK75 (which provides the gene for the die ompA signal sequence in the same reading frame 5′ to the Stu I recognition sequence) and restricted with the endonucleases Stu I and Sal I. After removal of the enzymes (PCR removal kit, Qiagen) the DNA fragments formed were covalently linked to T4 ligase by incubation. After removal of the T4 ligase (PCR removal kit, Qiagen) the undesired ligation products formed in detectable quantities were linearized by treatment with the endonucleases Eco RI (recognition sequence in the polylinker of pASK75 between the Stu I- and Sal I recognition sequences) and Cla I (recognition sequence in vector pT7) prior to transformation of E. coli XL1 Blue. The DNA was prepared from 5 ml “overnight” cultures of selected XL1 Blue clones which had grown after plating the transformation mixture on ampicillin selective agar (Qia-Prep Kit, Qiagen). In FIG. 11.e, the exemplary arrangement of recognition sequences for restriction endonucleases as well as the translation stop codons TAG and TAA is shown which facilitates a secretory expression as well as an insertion of the modular gene cassette into a corresponding vector. By treatment with suitable restriction endonucleases and subsequent agarose gel electrophoresis clones with characteristic band patterns were identified which had intracellularly established the desired plasmid pIML-02-P (FIG. 16.2 bottom).

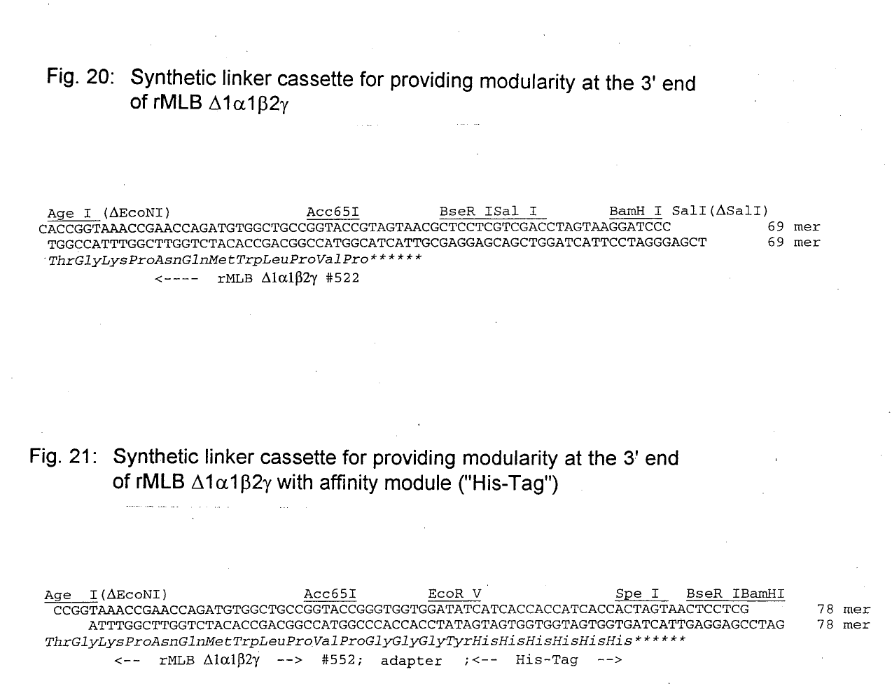

In order to provide modularity in the 3′ region of the modulator module corresponding synthetic gene fragments were cloned (FIG. 16.3 top). Equal volumina of synthetic oligonucleotides which were complementary to each other were heated in a concentration of 10 pmol/μl in a thermocycler for 1 min to 95° C. and hybridized by cooling down to 4° C. (3° C./min). The nucleotide sequences of the respective oligonucleotide pairs are such that DNA ends formed after hybridization are complementary to the DNA ends of the expression vectors which were treated with the corresponding restriction endonucleases (FIG. 16.3 middle). For this purpose, from vector pIML-02-P an about 100 bp 3′ region in the IMLB gene was excised using the endonucleases Age I and Bam HI (Age I and Sal I). Subsequent treatment of the solution with alkaline phosphatase (NEB) and removal of the enzymes (PCR removal kit) avoids the potential religation of the fragments during the subsequent ligation. In an T4 ligase reaction a gene fragment (FIG. 20) containing the amino acid sequence of rIMLB was fused to the Age I/Sal I restricted vector (pIML-02-P) and additionally the recognition sequences of the restriction endonucleases Acc 65I, Bse RI, Sal I and Bam HI were provided for the cloning of targeting domains (FIG. 16.3). In a second ligase reaction a further synthetic gene fragment having DNA ends which were complementary to the Age I, Bam HI restriction products of the vector, which beside the C terminal amino acids of rIMLB also encodes an affinity module (His-Tag) of the sequence (Gly)₃-Tyr-(His)₆ (SEQ ID NO: 48)_(FIG. 21), was likewise fused (FIG. 16.3 middle).

The thus obtained expression vectors pIML-03-P and pIML-03-H serve as starting constructs for the production of ITF-toxins which are generated therefrom by fusion with structural genes for the various targeting modules (FIG. 16.3 bottom). The targeting modules may be inserted by way of the existing restriction sites before or behind each module (effector, processing, modulator, affinity module; FIG. 17).

### EXAMPLE 11

### Construction of an ITF Variant with Toxicity Vis-à-Vis a Neuritogenic T Cell Line

In a selected example an ITF toxin is constructed to kill a P2 reactive human T cell line (Weishaupt et al., 1995) which contains as targeting module a synthetic DNA sequence encoding a fragment of 26 amino acids (aa 53-78) of the P2 protein (component of the myelins in the peripheral nervous system; FIG. 19) between modulator and affinity module of the vector pIML-03-H (FIG. 17 left bottom). For this purpose vector pIML-03-H—in analogy to the method described in Example 10—was restricted with Acc 651 and Eco RV, dephosphorylated, purified and ligated in the presence of T4 ligase with the oligonucleotides hybridized earlier. After transformation of the ligation mixture in E. coli XL1 Blue the plasmid DNA of selected clones which proliferate on ampicillin selective agar was examined by way of the restriction endonuclease Eco RI for the presence of the targeting module (linearized vector in the agarose gel electropherogram). The sequence of selected plasmids with positive restriction map was then verified by nucleotide sequence analysis (FIG. 18).

### EXAMPLE 12

### Provision of ITF Toxins by Way of the Example of ITF-P2-C1

(12.a) Expression of pITF-P2-C1 in E. coli BL21

For the expression of pITF-P2-C1 a 50 ml pre-culture from a glycerol permanent culture was inoculated and cultivated up to the late logarithmic phase (25° C., 150 rpm). 10 ml each of this pre-culture were inoculated in 1000 ml LB/Amp medium (in 2000 ml aeration-causing flask). The growth of the culture was observed by turbidimetry at 578 nm. At an OD of 1.0 the expression of the ITF-P2-C1 genes was induced by addition of 200 μM anhydrotetracycline. For monitoring the course of expression equal cell amounts were taken every 30 min starting from the time of induction and boiled in sample buffer (10% SDS, 200 mM DTT, 50 mM Tris/HCl, pH 6.8) and analyzed in a Western blot (FIG. 26). After an induction time of two hours the cells were sedimented (20 min, 6000 rpm, 4° C., JA20 Rotor), resuspended in 20 ml/1 culture volume disruption buffer (600 mM NaCl, 10 mM imidazole, 10% (v/v) glycerol, 50 mM Na₂HPO₄, pH 8.0) and then broken up by an N₂ gas pressure homogenizer (1×1500 psi) and subsequent ultrasonification (2 min, 50 W, 50% pulse time). Then the soluble fraction was separated from the insoluble components by centrifugation (45 min, 20000 rpm, 4° C., JA20 Rotor).

(12.b) Functionality of the Affinity Module Under Native Conditions by Way of the Example of the Enrichment of ITF-P2-C1 from the Soluble Fraction of E. Coli Extracts

ITF-P2-C1 solubly accumulated during expression in E. coli can be enriched on nickel Nta sepharose by affinity chromatography. For this purpose, an extract of soluble E. coli proteins is prepared (see Example 12.a). 40 ml of this protein solution are incubated while agitating for 30 min at 4° C. after 1 ml column material was added (Ni-NTA sepharose, Qiagen). Then the column matrix was washed 2× with 5 ml washing buffer (600 mM NaCl, 20 mM imidazole, 10% (v/v) glycerol, 50 mM Na₂HPO₄, pH 8.0). Bound protein was then eluted with elution buffer (600 mM NaCl, 250 mM imidazole, 10% (v/v) glycerol, 50 mM NaH₂PO₄, pH 6.5). The eluted fractions were then examined for their ITF content in a Western blot (FIG. 25), selected fractions were pooled, concentrated to a volume of 2 ml and dialyzed against storage buffer (500 mM NaCl, 10% (v/v) glycerol, 0.1 g/l PVP, 20 mM Na₂HPO₄, pH 7.6). The ITF content of the solution thus obtained was determined by Western blot analysis using an nML1 reference sample of known concentration.

(12.c) Functionality of the Affinity Module Under Denaturing Conditions by Way of the Example of the Enrichment of ITF-P2-C1 from the Insoluble Fraction of E. Coli Extracts

The ITF-containing inclusion bodies which were contained in the sediment of an E. coli complete cell disruption (see Example 12.a) were dissolved by 12 hrs of incubation with 1 ml/denaturing buffer (7 M GuHCl, 50 mM Na₂HPO₄, pH 8.0) and simultaneous denaturation. Insoluble cell components were sedimented by centrifugation (1 hr, 20000 rpm, 4° C., JA20 Rotor). For an enrichment of ITF-P2-C1 the soluble supernatant was incubated 2 hours with 1 ml affinity matrix (Ni-NTA sepharose, Qiagen) while agitating, the column material was washed with 2×5 ml washing buffer (7 M GuHCl, 50 mM NaH₂PO₄, pH 6.3) and bound protein was eluted with 4 ml elution buffer 1 (7 M GuHCl, 50 mM NaH₂PO₄, pH 4.5) and 4 ml elution buffer 2 (7 M GuHCl, 250 mM imidazole, 50 mM NaH₂PO₄, pH 4.5). The ITF content of the thus obtained guanidinium chloride solution was then determined by Western blot analysis using the monoclonal antibody TB33 by way of an nML1 sample of known concentration (FIG. 24).

### (12.d) Process for the Production of ITF Toxin by In Vitro Folding

Solubly folded ITF is produced by slowly adding dropwise an ITF-containing GuHCl solution into the 90-fold volume folding buffer (50 mM KCl, 1 mM EDTA, 100 mM glucose, 10 mM lactose, 10% (v/v) glycerol, 5 mM glutathion red., 1 mM glutathion ox., 50 mM Tris/HCl, pH 8.5) under 12 hrs' stirring at 4° C. Subsequently, insoluble components were sedimented by centrifugation (45 min, 20000 rpm, 4° C., JA20 Rotor) and the supernatant concentrated by factor 100. After dialysis against the 1000-fold volume storage buffer (500 mM NaCl, 10% (v/v) glycerol, 0.1 g/l PVP, 20 mM Na₂HPO₄, pH 7.6) soluble, active ITF is obtained (FIG. 27). The concentration of soluble ITF can be determined by Western blot analysis with monoclonal antibodies against nMLB (TB33) using a reference sample of known nIML content.

### EXAMPLE 13

### Determination of the Cytotoxicity of ITF-P2-C1 Vis-à-Vis P2-Specific T Cells

The neuritogenic P2-specific cell line G7TC (Weishaupt et al., 1997) from a female Lewis rat was cultivated in RPMI 1640 medium with 1% rat serum. After the cells had thawed, the living cells were counted, a cell suspension in a density of 500 000 cells/ml was prepared and the cells were seeded in plates with 6 wells in a volume of 2.5 ml per well. Treatment with the ITF construct P2-C1 (the P2 peptide and the affinity module are fused C terminally to the pro-ML with inactivated carbohydrate binding sites). Treatment was carried out for 2 hrs or for 24 hrs at 37° C. and 5% CO₂ at a vapor saturation with maximum 1/25 volume of the test substance dilution or the same volume buffer. A concentration of the ITF-P2-C1 of 50 ng/ml yields the end concentrations of 1, 1.5 and 2 ng/ml with the selected volumina of 50, 75 and 100 μl in 2.5 ml culture volume. For the detection of the cytotoxicity (apoptosis and necrosis) a fluorescence staining with subsequent flow cytometry is carried out. The principle is based on the binding of FITC-labeled annexin V to phosphatidylserine which is translocated to the outer side in membranes of apoptotic cells. Additionally those cells are stained by DNA-binding propidiumiodide which due to a toxic effect (direct necrosis, secondary necrosis after apoptosis) exhibit an increased membrane permeability, i.e., apoptotic cells are labeled with FITC (green fluorescence) while necrotic cells are stained twice or exhibit only PI-stain (red fluorescence). The staining was carried out following the instructions of the commercially available kits with 100 μl cell suspension each. The incubation of P2-specific T cells with the ITF resulted after 2 hrs in an increase of the apoptotic cells at 1 ng/ml to the threefold of the buffer control (FIG. 28.a LR vs. 28.b LR) while at 2 ng/ml a shift to necrotic cells was observed (FIG. 28.a UL vs. 28.c UL). After 24 hrs a drastic effect regarding the increase of the share of necrotic cells from 4% in the control to 16.6% was noted (FIG. 29.a UL vs. 29.d UL). At 1 ng/ml, however, a slight increase of the number of apoptotic cells (2.7 to 3.8%) is measured (FIG. 29.a LR vs. 29.b LR). It can be noted that the ITF on the basis of mistletoe lectin—as expected according to the invention—has the two effects on immune cells which are described for this plant toxin.

### ABBREVIATIONS

The following abbreviations are used herein.
A affinity module
bFGF basic fibroblast growth factor
DTT dithiothreitol
E effector module
EDTA ethylenediamine tetraacetate
GFP Green Fluorescent Protein
IgE immunoglobulin E
IgG immunoglobulin G
IL-2 interleukin 2
IPTG isopropylthiogalactoside
ITF immuno-targeted fusion proteins
M modulator module
MHC main histocompatibility complex
P processing module
PAGE polyacrylamide gel electrophoresis
ProML pro-mistletoe lectin
RIP ribosome-inactivating protein
(r)ML (recombinant) mistletoe lectin
(r)MLA (recombinant) mistletoe lectin A chain
(r)MLB (recombinant) mistletoe lectin B chain
nMLA natural mistletoe lectin A chain
nMLB natural mistletoe lectin B chain
SPDP N-succinimidyl-3-(2-pyridyldithio-)propionate
T targeting module

Conventional abbreviations are used for amino acids.

The following are the complete citations for references cited in this application.
Babbitt, P. C., West, B. L., Buechter, D. D., Kuntz, I. D. and Kenyon, G. L. (1990): Removal of a proteolytic activity associated with aggregates formed from expression of creatine kinase in Escherichia coli leads to improved recovery of active enzyme. Bio/Technology 8, 945-949.
Battelli, M. G., Barbieri, L. and Stirpe, F. (1990). Toxicity of, and histological lesions caused by, ribosome-inactivating proteins, their IgG-conjugates, and their homopolymers. Acta Pathol. Microbiol. Scand. 98, 585-593.
Barbieri, L., Battelli, M. G., Stirrpe, F. (1993): Ribosome-inactivating proteins from plants. Biochim. Biophys. Acta 1154, 237-282.
Benatti, L., Saccardo, M. B., Dani, M., (1989): Nucleotide sequence of cDNA coding for saporin-6, a type-I ribosome-inactivating protein from Saponaria officinalis. Eur. J. Biochem. 183, 465-470.
Better, M., Bernhard, S. L., Williams R. E. et al. (1995) T_cell targeted immunofusion protein from Escherichia coli. J. Biol. Chem. 270, 25, 14951-57.
Bolognesi, A., Tazzari, P. L., Tassi, C., Gromo, G., Gobbi, M. and Stirpe, F. (1992): A comparison of anti-lymphocyte immunotoxins containing different ribosome-inactivating proteins and antibodies. Clin. Exp. Immunol. 89, 341-346.
Brinkmann, U. (1996): Recombinant immunotoxins: protein engineering for cancer therapy. Mol. Med. Today, 10, 439-446.
Bussing, A., Suzart, K., Bergmann, J., Pfüller, U., Schietzel, M., Schweizer, K. (1996): Induction of apoptosis in human lymphocytes treated with Viscum album L. is mediated by the mistletoe lectins. CANCER LETTERS, 99, 59-72.
Byers, V. S., Rodvien, R., Grant, K. (1989): Phase I study of monoclonal antibody-ricin A chain immunotoxin xXomaZyme 791 in patients with metastatic colon cancer. Cancer Res. 49, 6153-6160.
Carlsson, J., Drevin, H., Axen, R. (1978): protein thiolation and reversible protein-protein conjugation. N-Succinimidyl-3-(2-pyridyldithio)propionate, a new heterobifunctional reagent. Biochem. J. 173, 723-737.
Collier, R. J. (1988): Structure activity relationships in diphteria toxin and Pseudomonas aeruginosa exotoxin. A. Cancer Treat Res. 37, 25-35
Cook, J. P., Savage, P. M., Lord, J. M., Roberts, L. M (1993): Biologically active Interleukin 2-ricin A fusion proteins may require intracellular proteolytic cleavage to exhibit a cytotoxic effect.
Deng, W. P. and Nickoloff, J. A. (1992): Site-directed mutagenesis of virtually any plasmid by eliminating a unique site. Anal. Biochem. 200, 81-88.
Frankel, A. E., Burbage, C., Fu, T., Tagge, E., Chandler, J. and Willingham, M. C. (1996): ricin toxin contains at least three galactose-binding sites located in B chain subdomains 1 alpha, 1 beta, and 2 gamma. Biochemistry 26, 14749-14756.
Gabius, H. J., Walzel, H., Joshi, S. S., Kruip, J., Kojima, S., Gerke, V., Kratzin, H., Gabius, S. (1992): The immunomodulary β-galactoside-specific lectin from mistletoe: partial sequence analysis, cell and tissue binding, and impact on intracellular biosignalling of monocytic leukemia cells. Anticancer Res. 12, 669-676.
Gary, G. L., Smith, D. H., Baldridge, J. S. (1983): Cloning, nucleotide sequence and expression in Escherischia coli of the exotoxin A structural gene of Pseudomonas aeruginosa. Proc. Natl. Acad. Sci. USA, 8, 2645-2649.
Glennie, M. J., Brennand, D. M., Bryden, F., McBride, H. M., Stirpe, F., Worth, A. T., Stevenson, G. T. (1988): Bispecific F(ab′ gamma)₂ antibody for the delivery of saporin in the treatment of lymphoma. J. Immunol. 141, 3662-3670.
Gold, R., Hartung, H.-P., and Lassmann, H. (1997) T_cell apoptosis in autoimmune diseases: termination of inflammation in the nervous system and other sites with specialized immune-defense mechanisms. TINS 20, 399-403.
Gould, B. J., Borowitz, M. J., Groves, E. S. (1989): Phase I study of an anti-breast cancer immunotoxin by continuous infusion: report of a targeted toxic effect not predicted by animal studies. J. Natl. Cancer Inst. 81, 775-781.
Greenfield, L., Bjorn, M. J., Horn, G. (1983): Nucleotide sequence of the structural gene for diphteria toxin carried by corynebacteriophage β. Proc. Natl. Acad. Sci. USA, 80, 6853-6857.
Hara-Nishimura, I., Inoue, K., Nishimura, M. (1991): A unique vacuolar processing enzyme responsible for the conversion of several proprotein precursors into the mature forms. FEBS Lett. 294, 89-93.
Harlowe, E., Spur, D. (1988) In: Antibodies. A laboratory manual. Cold Spring Harbor Laboratory, Cold Spring Harbor, N.Y.
Hohlfeld, R. (1997) Biotechnological agents for the immunotherapy of multiple sclerosis. Brain, 120, 865-916.
Jansen, F. K., Blythman, H. E., Carriere, D., Casellas, P., Gros, O., Gros, P., Laurent, J. C., Paolucci, F., Pau, B., Poncelet, P., Richer, G., Vidal, H., Voisin G. A. (1982): Immunotoxins: hybrid molecules combining high specificity and potent cytotoxicity. Immunol. Rev. 62, 185-216.
Janssen, O., Scheffler, A., & Kabelitz, D. (1993). in vitro effects of mistletoe extracts and mistletoe lectins. Cytotoxicity towards tumor cells due to the induction of programmed cell death (apoptosis). Drug Res., 43, 1221-7.
Lamb, F. I., Roberts, L. M., Lord, J. M. (1985): Nucleotide sequence of cloned cDNA coding for preproricin. Eur. J. Biochem. 148, 265-270.
Lambert, J. M., Blättler, W. A., McIntyre, G. D., Goldmacher, V. S, and Scott, C. F. Jr. (1988) In: Immunotoxins (Frankel, A. E. Hrsg.) pp 175-209, Kluwer, Boston.
Lappi, D. A., Baird, A. (1990): Mitotoxins: growth factor-targeted cytotoxic molecules. Prog. Growth Fact. Res. 2, 223-236.
Lappi, D. A., Ying, W., Barthelemy, I., Martineau, D., Prieto, I., Benatti, L., Soria, M., Baird, A. (1994): expression and activities of a recombinant basic fibroblast growth factor-saporin fusion protein. J. Mol. Biol. 17, 12552-12558.
Lehar, M. S., Pedersen, J. T., Kamath, R. S., Swimmer, C., Goldmacher, V. S., Lambert, J. M., Blättler, W. A. and Guild, B. C. (1994): mutational and structural analysis of the lectin activity in binding domain 2 of ricin B chain. Prot. Engineering 7, 1261-1266.
Lord, J. M. (1985): Precursors of ricin and ricinus communis agglutinin: glycosylation and processing during synthesis and intracellular transport. Eur. J. Biochem. 146, 411-416.
Magnusson, S., Berg, T. (1993): Endocytosis of ricin by rat liver cells in vivo and in vitro is mainly mediated by mannose receptors on sinusoidal endothelial cells. Biochem. J. 291, 749-755.
Masuho, Y., Kishida, K., Saito, M., Umemoto, N., Hara, T. (1982): Importance of the antigen-binding valency and the nature of the cross-linking bond in ricin A-chain conjugates with antibody. J. Biochem. 91, 1583-1591.
Milstein, C., Cuello, A. C. (1983): Hybrid hybridomas and their use in immunohistochemistry. Nature 305, 537-540.
Mosmann, T., (1983). Rapid colorimetric assay for cellular growth and survival: application to proliferation and cytotoxicity assays. J. Immunol. Methods 65, 55-63.
Möckel, B., Schwarz, T., Zinke, H., Eck, J., Langer, M., and Lentzen, H. (1997) Effects of mistletoe Lectin I on Human Blood Cell Lines and Peripheral Blood Cells. Drug Res. 47, 1145-51.
Nicolson, G. L. (1974): Ultrastructural analysis of toxin binding and entry into mammalian cells. Nature 251, 628-630.
Olive, C. (1995): T cell receptor usage in autoimmune disease. Immunol. Cell. Biol. 73, 297-307.
Paprocka, M., Wiedlocha, A., Walzel, H., Radzikowski, C. (1992): The activity of two immunotoxins composed of monoclonal antibody MoAb-16 and A-chain of ricin (MoAb-16-RTA) or A-chain of mistletoe lectin I (MoAb-16-MLIA). Archivum Immunologiae et Therapiae Experimentalis, 40, 223-227.
Pastan, I., FitzGerald, D. (1989): Chimeric toxins. J. Biol. Chem. 264, 15157-15160.
Pastan, I., FitzGerald, D. (1991): Recombinant toxins for cancer treatment. Science 254, 1173-1177.
Pastan, I., Chaudhary, V., FitzGerald, D. J. (1992): Recombinant toxins as novel therapeutic agents. Annu. Rev. Biochem. 61, 331-354
Price, V. L. (1996) fusion proteins comprising GM-CSF and antigens and their expression in yeast. International Patent, C12N 15/62, 15/63, WO 96/01903.
Ramakrishnan, S., Fryxell, D., Mohanraj, D., Olson, M., Li, B. Y. (1992): Cytotoxic conjugates containing translational inhibitory proteins. Annu. Rev. Pharmacol. Toxicol. 32, 579-621.
Richardson, P. T., Westby, M., Roberts, M., Gould, J. H., Colman, A., Lord, J. M. (1989): Recombinant proricin binds galactose but does not depurinate 28S rRNA. FEBS Lett. 255, 15-20.
Rutenber, E., Ready., and Robertus., J. D. (1987): Structure and evolution of ricin B chain. Nature 326, 624-626.
Schmied, M., Breitschopf, H., Gold, R., Zischler, H., Rothe, G., Wekerle, H. et al., (1993) Apoptosis of T-lymphocytes in experimental autoimmune encephalomyelitis. Evidence for programmed cell death as a mechanism to control inflammation in the brain. Am. J. Pathol. 143, 446-52.
Scudiero, P. A. et al. (1988). Evaluation of a soluble tetrazolium/formazan assay for cell growth and drug sensitivity in culture using human and other tumor cell lines. Cancer Res. 48, 4827-4833.
Shah, S. A., Halloran, P. M., Ferris, C. A., Levine, B. A., Bourret, L. A., Goldmacher, V. S. and Blättler, W. A. (1993): Anti-B4-blocked ricin immunotoxin shows therapeutic efficacy in four different SCID mouse tumor models. Cancer Res. 53, 1360-1367.
Shen, G. -L., Li, J. -L., Ghetie, M. A. (1988): Evaluation of four CD22 antibodies as ricin A-chain containing immunotoxins for the in vivo therapy of human B_cell leukemias and lymphomas. Int. J. Cancer 42, 792-797.
Skilleter D. N., Price, R. J., Thorpe, P. E. (1985) Modification of the carbohydrate in ricin with metaperiodate and cyanoborohydride mixtures: effect on binding, uptake and cytotoxicity to parenchymal cells of rat liver. Biochim. Biophys. Acta 842, 12-21.
Spitler, L., del Rio, M., Khentigan, A. (987): Therapy of patients with malignant melanoma using a monoclonal antimelanoma antibody ricin-A chain immunotoxin. Cancer Res. 47, 1717-1723.
Soer-Rodriguez, A. M., Ghetie, M. A., Oppenheimer-Marks, N., (1993): ricin A-chain and ricin A-chain immunotoxins rapidly damage human endothelial cells: implications for vascular leak syndrome. Exp. Cell Res. 206, 227-234
Spack, E. G., McCutcheon, M., Corbelletta, N., Nag, B., Passmore, D., and Sharma, S. D. (1995) Induction of tolerance in experimental autoimmune myasthenia gravis with solubilized MHC class II:acetylcholine receptor peptide complexes. J. Autoimmun. 8, 787-807.
Stirpe, F., Barbieri, L., Battelli, M. G., Soria, M., Lappi, D. A. (1992): Ribosome-inactivating proteins from plants: present status and future prospects. Biotechnology 10, 405-412.
Sun, J. -B., Rask, C., Olsson, T., Holmgren, J., and Czerkinsky, C. (1996) Treatment of experimental autoimmune encephalomyelitis feeding myelin basic protein conjugated to cholera toxin B subunit. Proc. Natl. Acad. Sci. USA 93, 7196-7201.
Swimmer, C., Lehar, S. M., McCafferty, J., Chiswell, D. J., Blättler, W. A. and Guild, B. C. (1992): Phage display of ricin B chain and ist single binding domains: System for screening galactose-binding mutants. Proc. Natl. Acad. Sci. USA 89, 3756-3760.
Thorpe, P. E., Brown, A. N. F., Ross, W. C. J., Cumber, A. J., Detre, S. I., Edwards, D. C., Davies, A. J. S, and Stirpe, F. (1981): Blockade of the galactose-binding sites of ricin by its linkage to antibody. Specific cytotoxic effects of the conjugates. Eur. J. Biochem. 116, 447-454.
Tonevitsky, A. G., Toptygin, Ayu., Pfüller, U., Bushueva, T. L., Ershova, G. V., Gelbin, M., Pfuller, K., Agapov, I. I., Franz, H. (1991): Immunotoxin with mistletoe lectin I A-chain and ricin A-chain directed against CD5 antigen of human T-lymphocytes; comparison of efficiency and specificity. International Journal of Immunopharmacology, 13, 1037-41.
Tonevitsky, A. G., Rakhmanova, V. A., Agapov, I. I., Shamshiev, A. T., Usacheva, E. A., Prokophév, S. A., Denisenko, O. N., Alekseev, Y. and Pfueller, U. (1995): The interactions of anti-ML1 monoclonal antibodies with isoforms of the lectin from Viscum album. Immunol. Lett. 44, 31-34.
Tonevitsky, A. G., Agapov, I. I., Shamshiev, A. T., Temyakov, D. E., Pohl, P., Kirpichnikov, M. P. (1996): Immunotoxins containing A-chain of mistletoe lectin I are more active than immunotoxins with ricin A-chain. FEBS Letters, 392, 166-168.
Vandenbark, A. A. (1996) Treatment of multiple sclerosis with T_cell receptor peptides: results of a double-blind pilot trial. Nature Med. 2, 1109-1115.
Vang, O., PiiLarsen, K., Bog-Hansen, T. C. (1986): A new quantitative and highly specific assay for lectin binding activity. In: Bog-Hansen, T. C., van Driessche, E. (Hrsg.) Lectins: Biology, Biochemistry, Clinical Biochemistry, Vol. 5, De Gruyter, Berlin, S. 637-644.
Vitetta, E. S., Fulton, R. J., May, R. D., Till, M., Uhr, J. W. (1987): Redesigning nature's poisons to create anti-tumor reagents. Science 228, 1098-1104.
Vitetta, E. S., Thorpe, P. E. (1991): Immunotoxins containing ricin or its A chain. Semin. Cell Biol. 2, 47-58.
Vitetta, E. S., Thorpe, P. E., Uhr, J. W. (1993): Immunotoxins: magic bullets or misguided missiles? Immunol. Today, 14, 252-259.
Vitetta, E. and Yen, N. (1990): expression and functional properties of genetically engineered ricin B chain lacking galaktose-binding activity. Biochim. Biophys. Acta 1049, 151-157.
Wales, R., Roberts, L. M., Lord, J. M. (1993): Addition of an endoplasmatic reticulum retrival sequence to ricin A chain significantly increases its cytotoxicity to mammalian cells. J. Biol. Chem. 268, 23986-23990.
Webb, K. S., Ware, J. L., Parks, S. F., Walther, P. J., Paulson, D. F. (1985): Evidence for a novel hybrid immunotoxin recognizing ricin A-chain by one antigen-combining site and a prostate-restricted antigen by the remaining antigen-Cancer Treat. Rep. 69, 663-672.
Weishaupt, A., Gierich, G., Jung, S., Gold, R., Enders, U., Pette, M., Hayasaka, K., Hartung, H. -P. and Toyka, K. V. (1995): T cell antigenic and neuritogenic activity of recombinant human peripheral myelin P2 protein. J. Neuroimmun. 63, 149-156.
Weishaupt, A., Gold, R., Gaupp, S., Giegerich, G., Hartung, H.-P., and Toyka, K. V. (1997) Antigen therapy eliminates T cell inflammation by apoptosis: Effective treatment of experimental autoimmune neuritis with recombinant myelin protein P2. Proc. Natl. Acad. Sci. USA 94, 1338-43.
Westby, M., Argent, R. H., Pitcher, C., Lord, J. M., Roberts, L. M. (1995): Preparation and characterization of recombinant proricin containing an alternative protease-sensitive linker sequenz. Bioconjugate Chem. 3, 375-381

We claim:

1. A nucleic acid molecule encoding a fusion protein which comprises
(a) an effector module which is intracellularly cytotoxic, the effector module comprising a mistletoe lectin A chain, wherein the mistletoe lectin A chain is encoded by a nucleic acid molecule selected from the group consisting of:
(i) a nucleic acid molecule which has a nucleotide sequence encoding a protein having the amino acid sequence SEQ ID NO:2; and
(ii) a nucleic acid molecule having the nucleotide sequence SEQ ID NO:1;
(b) a processing module which is covalently linked to the effector module and which comprises a recognition sequence for a protease, wherein the processing module comprises a mistletoe lectin propeptide, and wherein the mistletoe lectin propeptide is encoded by a nucleic acid molecule selected from the group consisting of:
(i) a nucleic acid molecule which has a nucleotide sequence encoding a protein having the amino acid sequence SEQ ID NO:6; and
(ii) a nucleic acid molecule having the nucleotide sequence SEQ ID NO:5; and
(c) a targeting module which is covalently linked to the processing module and which specifically binds to the surface of a cell, thereby mediating internalization of the fusion protein into the cell.

2. The nucleic acid molecule of claim 1, wherein the effector module possesses the biological activity of the mistletoe lectin A chain.

3. The nucleic acid molecule according to claim 1, wherein the processing module is proteolytically cleavable.

4. The nucleic acid molecule of claim 1, wherein the fusion protein further comprises a modulator module which is covalently linked to one of the processing module, the effector module, and the targeting module, wherein the modulator module modulates the intracellular cytotoxicity of the effector module, and wherein the modulator module is encoded by a nucleic acid molecule selected from the group consisting of a mistletoe lectin B chain, a nucleic acid molecule having a nucleotide sequence which encodes a protein having the amino acid sequence SEQ ID NO:4, a nucleic acid molecule which has the nucleotide sequence SEQ ID NO:3 and a nucleic acid molecule having a nucleotide sequence which encodes a protein having the amino acid sequence SEQ ID NO:4 with at least one amino acid substitution selected from the group consisting of an amino acid substitution of A at position D23, substitution of A at position W38, substitution of A at position D235, substitution of A at position Y249, substitution of S at position Y68, substitution of S at position Y70, substitution of S at position Y75, and substitution of S at position F79.
(i) a nucleic acid molecule having a nucleotide sequence which encodes a protein having the amino acid sequence SEQ ID NO:4; and
(ii) a nucleic acid molecule which has the nucleotide sequence SEQ ID NO:3.

5. The nucleic acid molecule of claim 4, wherein the modulator module possesses the biological activity of a mistletoe lectin B chain.

6. The nucleic acid molecule of claim 4, wherein the fusion protein further comprises an affinity module which is covalently linked to one of the effector module, the processing module, the targeting module, and the modulator module, wherein the affinity module comprises a peptide sequence having a ligand binding specificity or epitopes suitable for selective purification by an affinity chromatography method.

7. The nucleic acid molecule of claim 6, wherein the affinity module comprises a portion selected from the group consisting of a histidine sequence, thioredoxin, maltose-binding protein, green fluorescent protein, SEQ ID NO:39, and an 11 amino acid T7 gene leader peptide.

8. The nucleic acid molecule of claim 4, wherein the modulator module has a portion comprising one of a mistletoe lectin B chain, peptide KDEL (SEQ ID NO:35), and peptide HDEL (SEQ ID NO:36).

9. The nucleic acid molecule of claim 8, wherein the mistletoe lectin B chain has an amino acid substitution selected from the group consisting of an amino acid substitution of A at position D23, substitution of A at position W38, substitution of A at position D235, and substitution of A at position Y249.

10. The nucleic acid molecule of claim 8, wherein the mistletoe lectin B chain has an amino acid substitution selected from the group consisting of an amino acid substitution of S at position Y68, substitution of S at position Y70, substitution of S at position Y75, and substitution of S at position F79.

11. The nucleic acid molecule of claim 1, wherein the processing module is of plant origin and has an amino acid sequence the sequence SSSEVRYWPLVIRPVIA (SEQ ID NO:37).

12. The nucleic acid molecule of claim 1, wherein the targeting module specifically recognizes a cell selected from the group consisting of a cell of the immune system, a tumor cell, and a cell of the nervous system.

13. The nucleic acid molecule of claim 12, wherein the cell of the immune system is a cell of the specific immune system.

14. The nucleic acid molecule of claim 13, wherein the cell of the specific immune system is a T cell.

15. The nucleic acid molecule of claim 14, wherein the T cell is a T_{H}2 cell.

16. The nucleic acid molecule of claim 12, wherein the cell of the immune system is a cell of the unspecific immune system.

17. The nucleic acid molecule of claim 1, wherein the nucleic acid molecule is DNA.

18. The nucleic acid molecule of claim 1, wherein the nucleic acid molecule is RNA.

19. A vector comprising a nucleic acid molecule of claim 1.

20. A non-human host which is transformed with a vector of claim 19.

21. The host of claim 20, wherein the host is a prokaryote.

22. The host of claim 21, wherein the prokaryote is selected from the group consisting of E. coli, Bacillus subtilis, and Streptomyces coelicolor.

23. The host of claim 20, wherein the host is a eukaryote.

24. The host of claim 23, wherein the eukaryote is selected from the group consisting of a Saccharomyces species, an Aspergillus species, a Spodoptera species, and Pichia pastoris.

25. A non-human host which comprises a nucleic acid molecule of claim 1.

26. A method for producing a fusion protein, the method comprising culturing a host of claim 25 and isolating the fusion protein from the host.

27. The nucleic acid molecule of claim 1, wherein the targeting module comprises an MHC-binding peptide.

28. The nucleic acid molecule of claim 1, wherein the processing module comprises a protease-sensitive domain corresponding to a C-terminal sequence section of a basic fibroblast growth factor (bFGF).

29. The nucleic acid molecule of claim 1, wherein the fusion protein is selected from the group consisting of bFGF-mistletoe lectin A chain (MLA) and bFGF-MLA coupled to recombinant mistletoe lectin B chain (rMLB).

30. The nucleic acid molecule of claim 1, wherein the targeting module specifically recognizes a tumor cell.

31. A kit, comprising at least one of (a) and (b):
(a) a vector which comprises the nucleic acid molecule of claim 1; and
(b) a vector which comprises the nucleic acid molecule of claim 1, wherein the fusion protein further comprises an affinity module which is covalently linked to one of the effector module, the processing module and the targeting module, and the modulator module, wherein the affinity module comprises a peptide sequence having a ligand binding specificity or epitopes suitable for selective purification by an affinity chromatography method;
the kit further comprising (c):
(c) a vector which comprises a nucleic acid molecule encoding a modulator which modulates the intracellular cytotoxicity of the effector module of (a) and/or (b), wherein the modulator is selected from the group consisting of a mistletoe lectin B chain, a nucleic acid molecule having a nucleotide sequence which encodes a protein having the amino acid sequence SEQ ID NO:4, a nucleic acid molecule which has the nucleotide sequence SEQ ID NO:3 and a nucleic acid molecule having a nucleotide sequence which encodes a protein having the amino acid sequence SEQ ID NO:4 with at least one amino acid substitution selected from the group consisting of an amino acid substitution of A at position D23, substitution of A at position W38, substitution of A at position D235, substitution of A at position Y249, substitution of S at position Y68, substitution of S at position Y70, substitution of S at position Y75, and substitution of S at position F79.
